(19) United States
(12) Patent Application Publication
(10) Pub. No.: US 2007/0213734 A1
(43) Pub. Date: Sep. 13, 2007

(54) Tissue modification barrier devices and methods

(76) Inventors: Jeffery L. Bleich, Palo Alto, CA (US); Steven A. Spisak, San Jose, CA (US); Vahid Saadat, Saratoga, CA (US); Ronald Leguidleguid, Fremont, CA (US); Jeffery Bleam, Boulder Creek, CA (US); Michael Drews, Palo Alto, CA (US); James Yurchenco, Palo Alto, CA (US)

Correspondence Address: BAXANO, INC. & TOWNSEND AND TOWNSEND AND CREW, TWO EMBARCADERO CENTER, EIGHTH FLOOR, SAN FRANCISCO, CA, 94111-3834, US

(21) Appl. No.: 11/405,859

(22) Filed: Apr. 17, 2006

Related U.S. Application Data
(63) Continuation-in-part of application No. 11/375,265, filed on Mar. 13, 2006.

(51) Int. Cl. A61B 17/16

(52) U.S. Cl. 606/79

(57) ABSTRACT

A method for preventing unwanted damage to tissue in a spine of a patient during a tissue modification procedure may involve: advancing at least a distal portion of at least one barrier member over at least one guide member into an epidural space of the patient's spine; positioning at least an expanded portion of the barrier member between target tissue and non-target tissue; and performing at least one tissue modification procedure on the target tissue, using at least one tissue modification device. Generally, at least part of the barrier member may be disposed between the tissue modification device and the non-target tissue to prevent unwanted damage to the non-target tissue. In various embodiments, at least part of a barrier member may be advanced through a sheath- or catheter-like delivery device and may either automatically expand or be expandable from a collapsed configuration to an expanded configuration.

### CROSS REFERENCE TO RELATED APPLICATIONS

The present application is a continuation-in-part of U.S. patent application Ser. No. 11/375,265, entitled “Methods and Apparatus for Tissue Modification,” filed on Mar. 13, 2006 (Attorney Docket No. 78117-200101), the entire disclosure of which is hereby incorporated by reference. PCT Patent Application Pub. No. PCT/US2005/037136, which was incorporated fully by reference in the above-referenced U.S. patent application Ser. No. 11/375,265, is also incorporated fully by reference herein.

### BACKGROUND OF THE INVENTION

1. Field of the Invention

The present invention relates to methods and apparatus for modifying tissue in a patient.

Many pathological conditions in the human body may be caused by enlargement, movement, displacement and/or a variety of other changes of bodily tissue, causing the tissue to press against (or “impinge on”) one or more otherwise normal tissues or organs. For example, a cancerous tumor may press against an adjacent organ and adversely affect the functioning and/or the health of that organ. In other cases, bony growths (or “bone spurs”), arthritic changes in bone and/or soft tissue, redundant soft tissue, or other hypertrophic bone or soft tissue conditions may impinge on nearby nerve and/or vascular tissues and compromise functioning of one or more nerves, reduce blood flow through a blood vessel, or both. Other examples of tissues which may grow or move to press against adjacent tissues include ligaments, tendons, cysts, cartilage, scar tissue, blood vessels, adipose tissue, tumor, hematoma, and inflammatory tissue.

One specific example of a condition caused by tissue impingement is spinal stenosis. Spinal stenosis occurs when neural tissue and/or vascular tissue in the spine become impinged by one or more structures pressing against them (“neural and/or neurovascular impingement”), causing one or more symptoms. This impingement of tissue may occur in one or more of several different areas in the spine, such as in the central spinal canal (the vertical passage through which the spinal cord and cauda equina extends), the lateral recesses of the spinal canal, or one or more intervertebral foramina (the openings through which nerve roots branching from the spinal cord pass).

For explanatory purposes, FIG. 1 is offered to show an approximate top view of a vertebra (one of the bones of the spinal column) with the cauda equina (the horsetail-shaped bundle of nerves that extends from the base of the spinal cord through the central spinal canal) shown in cross section and two nerve roots exiting the central spinal canal and extending through intervertebral foramina on either side of the vertebra. (FIG. 1 is not drawn to exact scale and is intended for exemplary purposes only. It should be emphasized here that the drawing figures appended to this application are not intended to be precisely anatomically correct and are provided for exemplary purposes to facilitate description.) The spinal cord and cauda equina run vertically along the spine through the central spinal canal, while nerve roots branch off of the spinal cord and cauda equina between adjacent vertebrae and extend through the intervertebral foramina.

One common cause of spinal stenosis is buckling and thickening of the ligamentum flavum (one of the ligaments attached to and connecting the vertebrae), as shown in FIG. 1. Buckling or thickening of the ligamentum flavum may impinge on one or more neurovascular structures, dorsal root ganglia, nerve roots and/or the spinal cord itself. Another common cause of neural and neurovascular compression within the spine is disease of one or more of the intervertebral discs (the malleable discs between adjacent vertebrae), which may lead to collapse, bulging or herniation of the disc. In FIG. 1, an intervertebral disc is shown with three solid-tipped arrows demonstrating how the disc might bulge or herniate into the central spinal canal to impinge upon the spinal cord, cauda equina and/or individual nerve roots. Other causes of neural and neurovascular impingement in the spine include: hypertrophy of one or more facet joints (also known as zygopophaseal joints, facet joints provide articulation between adjacent vertebrae—two vertebral facet superior articular processes are shown in FIG. 1); formation of osteophytes (bony growths or “bone spurs”) on vertebrae; spondylolisthesis (sliding of one vertebra relative to an adjacent vertebra); and (facet joint) synovial cysts. Disc, bone, ligament or other tissue may impinge on the spinal cord, the cauda equina, branching spinal nerves and/or blood vessels in the spine to cause loss of function, ischemia (shortage of blood supply) and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stensosis include systemic medications and physical therapy. Epidural steroid injections may also be utilized, but they do not provide ling lasting benefits. When these approaches are inadequate, current treatment for spinal stenosis is generally limited to invasive surgical procedures to remove vertebral ligament, cartilage, bone spurs, synovial cysts, cartilage, and bone to provide increased room for neural and neurovascular tissue. The standard surgical procedure for spinal stenosis treatment includes laminectomy (complete removal of the lamina (see FIG. 1) of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints between vertebrae). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments.

While laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Therefore, it would be desirable to have less invasive methods and devices for addressing neural and neurovascular impingement in a spine. Ideally, methods and devices for addressing impingement in spine would treat one or more target tissues while preventing unwanted effects on adjacent or nearby non-target tissues. Also ideally, such methods and devices would be minimally invasive and reduce impingement without removing significant amounts of vertebral bone, joint, or other spinal support structures, thereby avoiding the need for spinal fusion and, ideally, reducing the long-term morbidity levels resulting from currently available surgical treatments. It may also be advantageous to have less invasive methods and devices for modifying target tissues in parts of the body other than the spine while preventing modification of non-target tissues. At least some of these objectives will be met by the present invention.

2. Description of Background Art

Flexible wire saws and chain saws, such as threadwire saws (T-saws) and Gigli saws, have been used since the late 1800s to saw through or file/abrade bone and other tissue in the human body. See, for example, Brunori A et al., “Celebrating the Centenial (1894-1994): Leonardo Gigli and H is Wire Saw,” J Neurosurg 82:1086-1090, 1995. An example of one such saw is described in U.S. Pat. No. 8250, issued to P. A. Stohlmann on Nov. 28, 1876. A description of using a T-saw to cut vertebral bone is provided in Kawahara N et al., “Recapping T-Saw Laminoplasty for Spinal Cord Tumors,” SPINE Volume 24, Number 13, pp. 1363-1370.

A method and apparatus for treating spinal stenosis is described in PCT Patent Application Pub. No. WO 01/08571. A surgical instrument for removing cartilage from a knee cavity is described in U.S. Pat. No. 3,835,859.

### SUMMARY OF THE INVENTION

In various embodiments, the present invention provides methods, apparatus and systems for modifying tissue in a patient. Generally, the methods, apparatus and systems may involve using an elongate, at least partially flexible tissue modification device having one or more tissue modifying members to modify one or more target tissues. The tissue modification device may be configured such that when the tissue modification member (or members) is in a position for modifying target tissue, one or more sides, surfaces or portions of the tissue modification device configured to avoid or prevent damage to non-target tissue will face non-target tissue. In various embodiments, during a tissue modification procedure, an anchoring force may be applied at or near either a distal portion or a proximal portion of the tissue modification device, either inside or outside the patient. Pulling or tensioning force may also be applied to the unanchored end of the device to urge the tissue modifying member(s) against target tissue. The tissue modifying members may then be activated to modify tissue while being prevented from extending significantly beyond the target tissue in a proximal or distal direction. In some embodiments, the tissue modifying members may be generally disposed along a length of the tissue modification device that approximates a length of target tissue to be modified.

By “applying an anchoring force,” it is meant that a force is applied to maintain a portion of a device, or the device as a whole, substantially stable or motion-free. Applying an anchoring force is, therefore, not limited to preventing all movement of a device, and in fact, a device to which an anchoring force is applied may actually move in one or more directions in some embodiments. In other embodiments, an anchoring force is applied to maintain a portion of a device substantially stable, while another portion of the device is allowed to move more freely. As will be described in further detail below, applying an anchoring force in one embodiment involves a user of a device grasping the device at or near one of its ends. In other embodiments, devices may use one or more anchoring members to apply an anchoring force. In a number of embodiments, an anchoring force may be applied with or against one or more tissues of a patient's body, and the tissue(s) may often move even as they apply (or help apply) the force. Thus, again, applying an anchoring force to a device does not necessarily mean that all motion of the device is eliminated. Of course, in some embodiments, it may be possible and desirable to eliminate all movement or substantially all movement of a device (or portion of a device), and in some embodiments anchoring force may be used to do so.

Methods, apparatus and systems of aspects of the present invention generally provide for tissue modification while preventing unwanted modification of, or damage to, surrounding tissues. Tensioning the tissue modification device by applying anchoring force at or near one end and applying tensioning or pulling force at or near the opposite end may enhance the ability of tissue modification members of the device to work effectively within a limited treatment space. Applying tensioning force to a predominantly flexible device may also allow the device to have a relatively small profile, thus facilitating its use in less invasive procedures and in other procedures in which alternative approaches to target tissue may be advantageous.

In some embodiments, the described methods, apparatus and systems may be used to modify tissue in a spine, such as for treating neural impingement, neurovascular impingement and/or spinal stenosis. In alternative embodiments, target tissues in other parts of the body may be modified.

In one aspect of the present invention, a method for preventing unwanted damage to tissue in a spine of a patient during a tissue modification procedure may involve: advancing a distal portion of a delivery device into an epidural space of the patient's spine; exposing at least a portion of at least one barrier member out of the distal portion of the delivery device, wherein at least a portion of the barrier member is changeable from a collapsed configuration in the delivery device to an expanded configuration outside the delivery device; positioning at least part of the exposed barrier member between target tissue and non-target tissue in the spine; and performing at least one tissue modification procedure on the target tissue, using at least one tissue modification device. In some embodiments, at least part of the barrier member may be disposed between the tissue modification device and the non-target tissue to prevent unwanted damage to the non-target tissue.

In another aspect of the present invention, a method for preventing unwanted damage to tissue in a spine of a patient during a tissue modification procedure may involve: advancing at least a distal portion of at least one barrier member over at least one guide member into an epidural space of the patient's spine; positioning at least an expanded portion of the barrier member between target tissue and non-target tissue; and performing at least one tissue modification procedure on the target tissue, using at least one tissue modification device. Again, in some embodiments, at least part of the barrier member may be disposed between the tissue modification device and the non-target tissue to prevent unwanted damage to the non-target tissue.

In another aspect of the present invention, a method for preventing unwanted damage to tissue of a patient during a tissue modification procedure may involve: advancing at least a distal portion of a delivery device into the patient and to a position between or adjacent target tissue and non-target tissue; advancing at least a distal portion of at least one barrier member over at least one guide member to a position between or adjacent target tissue and non-target tissue in the patient; exposing at least a portion of the at least one barrier member out of the distal portion of the delivery device, wherein at least a portion of the barrier member is changeable from a collapsed configuration in the delivery device to an expanded configuration outside the delivery device; and performing at least one tissue modification procedure on the target tissue, using at least one tissue modification device.

In yet another of the present invention, a barrier device for preventing unwanted damage to tissue in a spine of a patient during a tissue modification procedure may include: at least one shape changing portion changeable from a collapsed configuration, to facilitate passage into the spine, to an expanded configuration, to facilitate protection of non-target tissue; at least one elongate portion extending beyond the shape changing portion, the elongate portion having a low profile to facilitate passage of the barrier device into the patient and a length sufficient to extend from an opening on the patient's skin to an area at or near the spine; and at least one guide feature extending along at least a portion of the barrier to allow the barrier to be passed into the patient over at least one guide member. In some embodiments, the barrier device may have an overall length sufficient to pass from a first opening on the patient's skin, into an epidural space of the spine, and between target and non-target tissue.

In another embodiment of the present invention, a barrier device for preventing unwanted damage to tissue of a patient during a tissue modification procedure may include: at least one shape changing portion changeable from a collapsed configuration, to facilitate passage into the patient, to an expanded configuration, to facilitate protection of non-target tissue; at least one elongate portion extending beyond the shape changing portion, the elongate portion having a low profile to facilitate passage of the barrier device into the patient and a length sufficient to extend from an opening on the patient's skin to an area at or near target and non-target tissues; and at least one guide feature extending along at least a portion of the barrier to allow the barrier to be passed into the patient over at least one guide member. In some embodiments, the barrier device may have an overall length sufficient to pass from a first opening on the patient's skin and between the target and non-target tissues.

A system for preventing unwanted damage to tissue in a spine of a patient during a tissue modification procedure may include a barrier device, a barrier delivery device for facilitating passage of the barrier device into the spine, and at least one guide member over which the barrier is passable into the spine. In some embodiments, the barrier may include: at least one shape changing portion changeable from a collapsed configuration, to facilitate passage into the spine, to an expanded configuration, to facilitate protection of non-target tissue; at least one elongate portion extending beyond the shape changing portion, the elongate portion having a low profile to facilitate passage of the barrier device into the patient and a length sufficient to extend from an opening on the patient's skin to an area at or near the spine; and at least one guide feature extending along at least a portion of the barrier to allow the barrier to be passed into the patient over at least one guide member. In some embodiments, the barrier device may have an overall length sufficient to pass from a first opening on the patient's skin, into an epidural space of the spine, and between the target and non-target tissue.

These and other aspects and embodiments are described more fully below in the Detailed Description, with reference to the attached Drawings.

### BRIEF DESCRIPTION OF THE DRAWINGS

FIG. 1 is cross-sectional view of a spine, showing a top view of a lumbar vertebra, a cross-sectional view of the cauda equina, and two exiting nerve roots;

FIG. 2 is a cross-sectional view of a portion of a patient's back and spine, showing part of a vertebra and apparatus in place for modifying tissue according to one embodiment of the present invention;

FIG. 3A is a perspective view of a tissue modification device according to one embodiment of the present invention;

FIG. 3B is a perspective view of a portion of the tissue modification device of FIG. 3A;

FIG. 3C is a top view of the portion shown in FIG. 3B;

FIG. 3D is a side view of the portion shown in FIGS. 3B and 3C;

FIGS. 3E and 3F are cross-sectional views of a portion of the tissue modification device taken through lines A-A and B-B, respectively, shown in FIG. 3C;

FIG. 3G is a perspective view of a portion of the tissue modification device of FIGS. 3B-3F, shown with a blade of the device in a closed position according to one embodiment of the present invention;

FIG. 3H is a top view of the portion shown in FIG. 3G;

FIG. 3I is a side view of the portion shown in FIGS. 3G and 3H;

FIG. 4A is a perspective view of a tissue modification device according to one embodiment of the present invention;

FIG. 4B is a perspective view of a portion of the tissue modification device of FIG. 4A;

FIG. 4C is a close-up, perspective view of a portion of the tissue modification device of FIGS. 4A and 4B, showing a tissue modifying member according to one embodiment of the present invention;

FIGS. 5A-5D are cross-sectional views of a spine and demonstrate a method for using a tissue modification device according to one embodiment of the present invention;

FIG. 6A is a cross-sectional view of a portion of a patient's spine and back, with apparatus for modifying tissue in position for modifying spinal tissue and with a distal portion of the apparatus anchored outside the patient according to one embodiment of the present invention;

FIG. 6B is a cross-sectional view of a portion of a patient's spine and back, with apparatus for modifying tissue in position for modifying spinal tissue and with a distal portion of the apparatus anchored inside the patient according to one embodiment of the present invention;

FIGS. 7A-7S are cross-sectional views of a portion of a patient's spine and back, demonstrating a method for introducing apparatus for modifying spinal tissue to an area in the spine for performing the tissue modification according to one embodiment embodiment of the present invention;

FIGS. 8A-8F are cross-sectional views of a portion of a patient's spine and back, demonstrating a method for introducing apparatus for modifying spinal tissue to an area in the spine for performing the tissue modification according to an alternative embodiment of the present invention;

FIGS. 9A-9B are cross-sectional views of a portion of a patient's spine and back, demonstrating a method for introducing apparatus for modifying spinal tissue to an area in the spine for performing the tissue modification according to an alternative embodiment of the present invention;

FIG. 10A is a perspective view of a distal portion of an introducer sheath according to one embodiment of the present invention;

FIGS. 10B and 10C are perspective and cross-sectional views, respectively, of a tissue shield device according to one embodiment of the present invention; and

FIGS. 10D and 10E are perspective and cross-sectional views, respectively, of a tissue shield device according to an alternative embodiment of the present invention.

FIG. 11A is a perspective view of a mesh-type barrier device deploying from a sheath according to one embodiment of the present invention.

FIG. 11B is a top view of the mesh-type barrier device of FIG. 11A in its free state, prior to loading in a sheath.

FIG. 11C is a perspective view of a flexible tab-type barrier device deploying from a sheath according to an alternative embodiment of the present invention.

FIG. 11D is a top view of the flexible tab-type barrier device of FIG. 11C in its free state, prior to loading in a sheath.

FIG. 11E is a perspective view of a slit-type barrier device deploying from a sheath according to an alternative embodiment of the present invention.

FIG. 11F is a top view of the slit-type barrier device of FIG. 11E in its free state, prior to loading in a sheath.

FIG. 11G is a perspective view of a rib-type barrier device deploying from a sheath according to an alternative embodiment of the present invention.

FIG. 11H is a top view of the rib-type barrier device of FIG. 11G in its free state, prior to loading in a sheath.

FIG. 11I is a perspective view of a sheet-type barrier device deploying from a sheath according to an alternative embodiment of the present invention.

FIG. 11J is a top view of the sheet-type barrier device of FIG. 11I in its free state, prior to loading in a sheath.

FIG. 11K is a perspective view of a bar-type barrier device deploying from a sheath according to an alternative embodiment of the present invention.

FIG. 11L is a top view of the bar-type barrier device of FIG. 11K in its free state, prior to loading in a sheath.

FIG. 12A is a perspective view of a barrier device deployed by means of a slider according to one embodiment of the present invention.

FIG. 12B is a perspective view of a portion of the barrier device of FIG. 12A, in its free state.

FIG. 12C is a cross-sectional view of the barrier device of FIG. 12A through line C-C

FIG. 13A is a perspective view of a barrier device deployed by means of separable groove according to one embodiment of the present invention.

FIG. 13B is a perspective view of the distal tip of the barrier device of FIG. 13A, showing the device prior to deployment.

FIG. 14A is a perspective view of a barrier device with interdigitating teeth according to one embodiment of the present invention.

FIG. 14B is a magnified view of an interdigitating tooth of FIG. 14A;

FIG. 14C is a top view of the interdigitating teeth of the barrier device of FIG. 14A.

FIG. 15A is a perspective view of a barrier device with a tapered tip and guidewire according to one embodiment of the present invention.

FIG. 15B is a cross-sectional view of the barrier device of FIG. 15A, through line D-D.

FIG. 16A is a perspective view of a barrier device with a flexible frame according to one embodiment of the present invention.

FIG. 16B is a cross-sectional view of the barrier device of FIG. 16A, through line E-E, with a smooth barrier device stored in the sheath according to one embodiment of the present invention.

FIG. 16C is a cross-sectional view of the barrier device of FIG. 16A, through line E‘-E’, with a ruffled barrier device stored in the sheath according to an alternative embodiment of the present invention.

FIG. 16D is a cross-sectional view of the barrier device of FIG. 16A, through line E″-E″, with a rolled barrier device stored in the sheath according to an alternative embodiment of the present invention.

FIG. 16E is a cross-sectional view of the barrier device of FIG. 16A, through line F-F, at the middle of the smooth, stretched barrier device according to one embodiment of the present invention.

FIG. 16F is a cross-sectional view of the barrier device of FIG. 16A, through line F‘-F’ at the middle of the loose barrier device according to one embodiment of the present invention.

FIG. 16G is a cross-sectional view of the barrier device depicted in FIG. 16A, through line F″-F″ at the middle of the bag-like barrier device according to one embodiment of the present invention.

FIG. 17 is a perspective view of a barrier device with a corrugated shape according to one embodiment of the present invention.

FIG. 18 is a perspective view of a barrier device composed of compliant tubes according to one embodiment of the present invention.

FIG. 19 is a perspective view of a barrier device with a self-expanding frame according to one embodiment of the present invention.

FIG. 20A is a perspective view of a barrier device with a self-expanding frame that has supplemental push rods according to one embodiment of the present invention.

FIG. 20B is a top view of a push rod diverter according to one embodiment of the present invention.

FIG. 21 is a perspective view of a barrier device with an enlarged self-expanding frame according to one embodiment of the present invention.

FIG. 22A is a perspective view of a barrier device with a rolled barrier material on each arm of a self-expanding frame according to one embodiment of the present invention.

FIG. 22B is a perspective view of the barrier device of FIG. 22A with the material unrolled from each arm of the self-expanding frame according to one embodiment of the present invention.

FIG. 23 is a perspective view of a barrier device with an articulated mechanism to expand the frame.

FIG. 24A is a perspective view of a delivery sheath for delivering a barrier device according to one embodiment of the present invention.

FIG. 24B is a perspective view of a barrier device with a 4-bar linkage in a compact state according to one embodiment of the present invention.

FIG. 24C is a perspective view of the barrier device of FIG. 24B in an expanded state according to one embodiment of the present invention.

FIG. 24D is a perspective view of a barrier device with multiple 4-bar linkages in a compact state according to an alternative embodiment of the present invention.

FIG. 24E is a perspective view of the barrier device of FIG. 24D in a expanded state according to one embodiment of the present invention.

FIG. 25A is a perspective view of a barrier device with multiple 4-bar linkages, actuated by a central member, in a compact state according to one embodiment of the present invention.

FIG. 25B is a perspective view of the barrier device of FIG. 25A, actuated by a central member, in an expanded state according to one embodiment of the present invention.

FIG. 26A is a perspective view of a barrier device with flex-linkages according to one embodiment of the present invention.

FIG. 26B is a perspective view of a flex-linkage with a strain-relief loop according to one embodiment of the present invention.

FIGS. 26C-26E are a series of top views of a barrier device with flex-linkages under different loading configurations according to one embodiment of the present invention.

FIGS. 27A and 27B are perspective views of a woven tube barrier device in low-profile and expanded states, respectively, according to one embodiment of the present invention.

FIGS. 28A and 28B are perspective views of a flat woven barrier device in low-profile and expanded states, respectively, according to one embodiment of the present invention.

FIGS. 29A and 29B are perspective views of a barrier device with a pull-mechanism in low-profile and expanded states, respectively, according to one embodiment of the present invention.

FIG. 30A is a perspective view of a cylindrical housing for a barrier device in an un-deployed state according to one embodiment of the present invention.

FIG. 30B is a perspective view of the cylindrical housing of FIG. 30A and a barrier device deployed from the housing according to one embodiment of the present invention.

FIGS. 30C-30F are perspective views illustrating a method of deploying the barrier device of FIGS. 30A and 30B between a hard tissue structure and a soft tissue structure according to one embodiment of the present invention.

FIG. 31A is a perspective view of a woven wire barrier device in an elongated state according to one embodiment of the present invention.

FIG. 31B is an enlarged perspective view of a portion of the barrier device of FIG. 31A;

FIG. 31C is a perspective view of the barrier device of FIG. 31A in an expanded/shortened state according to one embodiment of the present invention.

FIGS. 32A-32C are perspective views of a hydrogel material barrier device in the process of unrolling/expanding after exposure to a fluid according to one embodiment of the present invention.

FIGS. 33A-33C are perspective and side views of a barrier device made from a plurality of curved elements according to one embodiment of the present invention.

FIG. 34A is a perspective view of a barrier device with thin, expandable flexure members shown in an un-expanded state according to one embodiment of the present invention.

FIG. 34B is a perspective view of the barrier device of FIG. 34A in an expanded state according to one embodiment of the present invention.

FIG. 34C is a side view of the expanded barrier device of FIG. 34B.

FIG. 35 is a perspective view of a delivery device containing an un-deployed barrier device according to one embodiment of the present invention.

FIGS. 35A and 35B are perspective and end-on views, respectively, of a deployed barrier device according to one embodiment of the present invention.

FIGS. 35C and 35D are perspective and end-on views, respectively, of a deployed barrier device according to an alternative embodiment of the present invention.

FIGS. 35E and 35F are perspective and end-on views, respectively, of a deployed barrier device according to an alternative embodiment of the present invention.

FIGS. 35G and 35H are perspective and end-on views, respectively, of a deployed barrier device according to an alternative embodiment of the present invention.

FIGS. 36A and 36B are perspective views of an inflatable bladder barrier device in deflated and inflated states, respectively, according to one embodiment of the present invention.

FIGS. 37A-37E are perspective views of a barrier device including an inflatable bladder containing particles, illustrating inflation and deflation of the device according to one embodiment of the present invention.

FIG. 37F is a perspective view of various particles which may be used in various embodiments of the barrier device of FIGS. 37A-37E.

FIGS. 38A-38C are perspective and cross-sectional views of a barrier device including a bladder with a foam element to affect the bladder shape after inflation according to one embodiment of the present invention.

FIG. 39A is a perspective view of a dual channel introducer and a wedge barrier device that expands the introducer according to one embodiment of the present invention.

FIGS. 39B and 39C are cross-sectional views of the dual channel introducer and the wedge barrier device of FIG. 39A, through Line H-H and Line I-I, respectively, according to one embodiment of the present invention.

FIG. 40A is a perspective view of a barrier device according to one embodiment of the present invention.

FIG. 40B is a top view of a portion of a barrier device according to an alternative embodiment of the present invention.

FIG. 40C is an end-on view of the barrier device of FIG. 40A.

FIG. 40D is an end-on view of the barrier device of FIG. 40B.

FIG. 41A is a perspective view of a barrier device made from sphere-like elements, shown in an un-expanded state according to one embodiment of the present invention.

FIG. 41B is a perspective view of the barrier device of FIG. 41A, shown in an expanded state.

FIG. 41C is a detailed perspective view of a portion of the barrier device of FIG. 41A.

FIG. 42 is a perspective view of a barrier device including a cover and a malleable wire.

FIGS. 43A and 43B are perspective views of a barrier device and a tissue modification device according to one embodiment of the present invention.

FIGS. 44A and 44B are perspective views of a barrier device and a tissue modification device according to an alternative embodiment of the present invention.

FIG. 45 is a perspective view of a barrier device and a tissue modification device according to an alternative embodiment of the present invention.

FIG. 46 is a perspective view of a barrier device and a tissue modification device according to an alternative embodiment of the present invention.

FIGS. 47A and 47B are end-on views of a barrier device according to alternative embodiments of the present invention.

FIGS. 47C-47E are end-on views of a barrier device guide member according to alternative embodiments of the present invention.

FIGS. 48A-48C are end-on views of a barrier device guide member according to alternative embodiments of the present invention.

FIGS. 48D-48G are end-on views of a barrier device according to alternative embodiments of the present invention.

FIGS. 49A and 49B are end-on views of a barrier device according to alternative embodiments of the present invention.

FIG. 50 is an end-on view of a barrier device and delivery device according to one embodiment of the present invention.

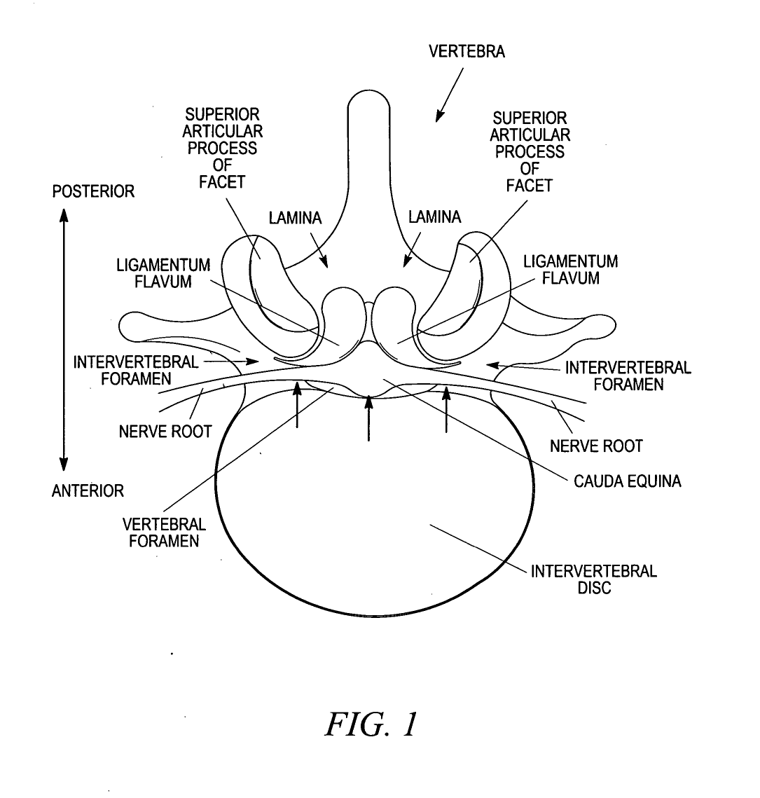

### DETAILED DESCRIPTION OF THE INVENTION

Methods, apparatus and systems for modifying tissue in a patient are provided. Although the following description and accompanying drawing figures generally focus on tissue modification in spine, in various alternative embodiments any of a number of tissues in any of a number of anatomical locations in a patient may be modified.

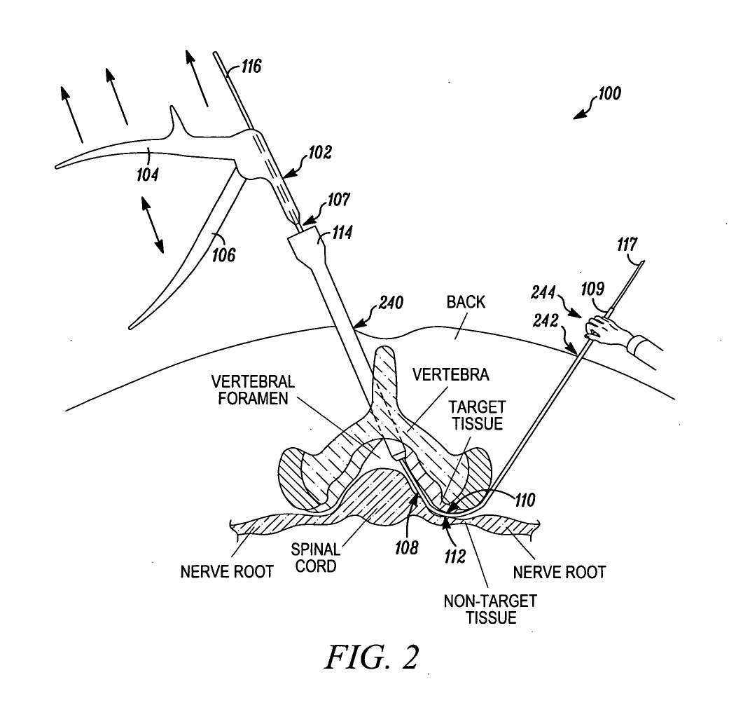

Referring to FIG. 2, in one embodiment a tissue modification device 102 may include an elongate body 108 having a proximal portion 107 and a distal portion 109, a handle 104 with an actuator 106 coupled with proximal portion 107, one or more tissue modifying members 110, and one or more protective surfaces 112. In various embodiments, some of which are described further below, modification device 102 may be introduced into an area for performing a treatment, such as a spine, using any of a number of different introduction methods, devices and systems. In FIG. 2, for example, modification device 102 extends through an introducer device 114 placed through a first incision 240 on the patient's back and into the central spinal canal. Modification device 102 is advanced along a guide member 116, which extends through introducer member 114, through the intervertebral foramen between two adjacent vertebrae (only part of one vertebra is shown in FIG. 2), and out a second (or “distal”) incision 242 on the back. In some embodiments, as shown, guide member has a beveled distal tip 117 for facilitating advancement of guide member 116 through tissue.

Generally, tissue modification device 102 may be advanced to a position in the spine such that tissue modifying member 110 faces target tissue to be modified, such as buckled, thickened or otherwise impinging ligamentum flavum tissue as shown in FIG. 2. Modification device 102 is configured such that when tissue modifying member 110 faces the target tissue, protective surface(s) 112 face non-target tissue. Protective surface 112 may be simply a length of elongate body 108 or may have one or more protective features, such as a widened diameter, protective or lubricious coating, extendable barrier, drug-eluting coating or ports, or the like. In some instances, protective surface(s) 112 may act as “non-tissue-modifying” surfaces, in that they may not substantially modify the non-target tissue. In alternative embodiments, protective surface(s) 112 may affect non-target tissue by protecting it in some active way, such as by administering one or more protective drugs, applying one or more forms of energy, providing a physical barrier, or the like.

In some embodiments, once tissue modification device 102 is positioned such that tissue modifying member 110 faces target tissue and protective surface 112 faces non-target tissue, an anchoring force may be applied at or near distal portion 109 of elongate body 108, either inside or outside the patient's body. A tensioning force may also be applied at or near proximal portion 107 of elongate body 108, such as by pulling on handle 104 (one-directional arrows), and actuator 106 may be used (two-headed arrow) to activate tissue modifying member(s) 110 to modify target tissue. In the example shown, anchoring force is applied near distal portion 109 by a user's hand 244, and handle 104 is pulled proximally (arrows) to apply tensioning force. In an alternative embodiment, hand 244 may grasp guide member 116 at or near its distal portion 117 and thus apply anchoring force to it, thus also applying anchoring force to elongate body 108. In one variation of such an embodiment, elongate body 108 or handle 104 may optionally be adjustably clamped to guide member 116 to further enhance or facilitate application of anchoring force to elongate body 108. Tissue modification via tissue modifying members 110 may include cutting, ablating, dissecting, repairing, reducing blood flow in, shrinking, shaving, burring, biting, remodeling, biopsying, debriding, lysing, debulking, sanding, filing, planing, heating, cooling, vaporizing, delivering a drug to, and/or retracting the target tissue. Once tissue has been modified, tissue modification device 102 and any introducer devices 114, guide members 116 or other devices may be removed from the patient.

In various embodiments of the apparatus, tissue modifying member(s) 110 may be disposed along any suitable length of body 108. In one embodiment, for example, such as an embodiment of the device to be used in a spinal treatment, tissue modifying members 110 may be disposed along a length of the device measuring no longer than 10 cm, and preferably no more than 6 cm, and even more preferably no more than 3 cm. In various embodiments, tissue modifying member(s) 110 may include a rongeur, a curette, a scalpel, one or more cutting blades, a scissors, a forceps, a probe, a rasp, a file, an abrasive element, one or more small planes, an electrosurgical device, a bipolar electrode, a unipolar electrode, a thermal electrode, a rotary powered mechanical shaver, a reciprocating powered mechanical shaver, a powered mechanical burr, a laser, an ultrasound crystal, a cryogenic probe, a pressurized water jet, a drug dispensing element, a needle, a needle electrode, or some combination thereof. In various embodiments, all tissue modifying members 110 may be mobile relative to the elongate body, all may be static, or some may be mobile and some may be static. These and other aspects and embodiments are described further below.

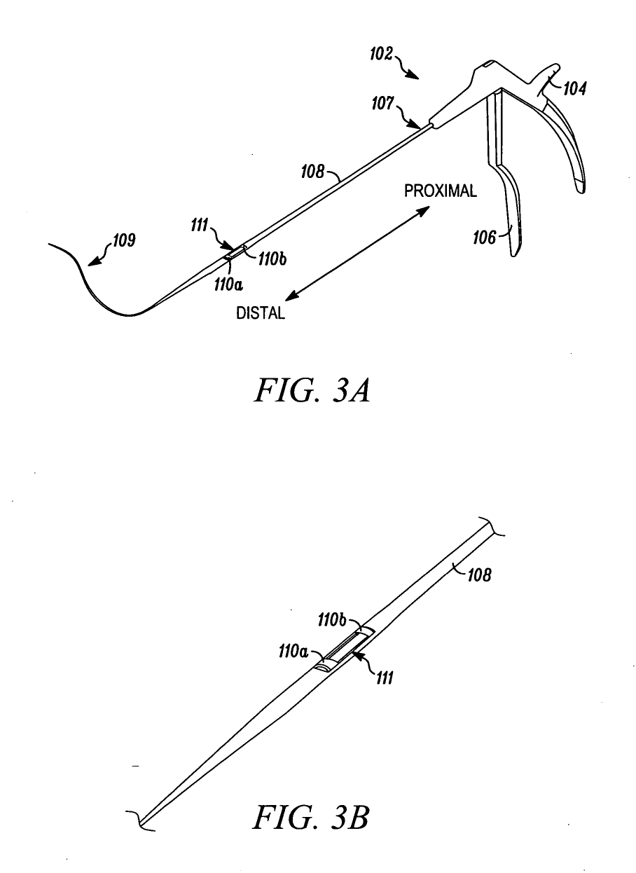

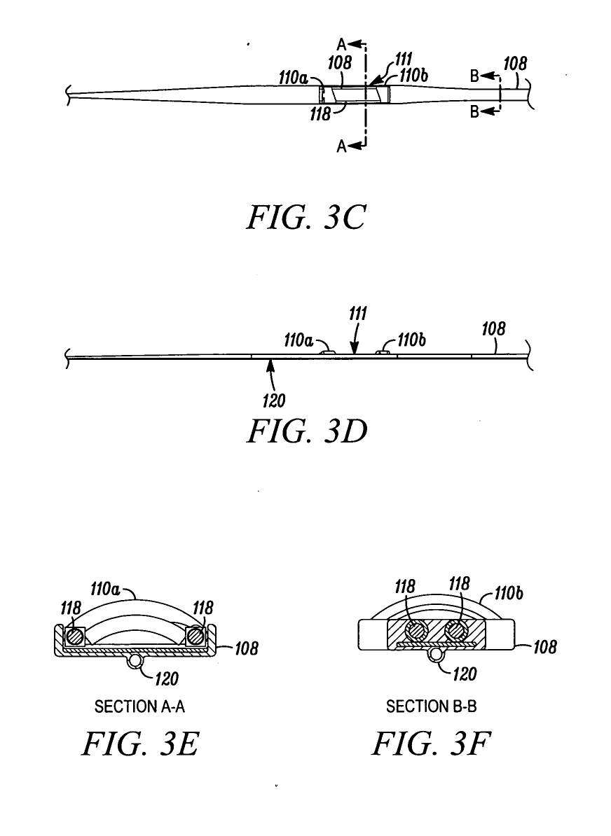

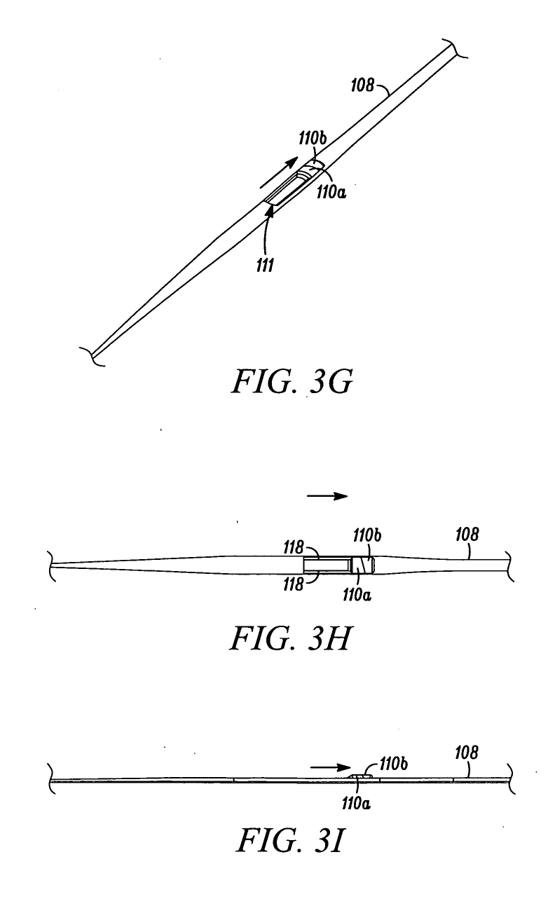

Turning now to FIG. 3A-3I, more detailed figures of one embodiment of tissue modification device 102 are shown. Referring to FIG. 3A, tissue modification device 102 may include elongate body 108 having proximal portion 107 and distal portion 109, a window 111 disposed along elongate body 108, two tissue modifying blades 110 exposed through window 111, and handle 104 with actuator 106 coupled with proximal portion 107. In the embodiment shown, the tissue modifying members comprise blades 110, although in alternative embodiments other tissue modifying members may be added or substituted.

In various embodiments, elongate body 108 may have any number of dimensions, shapes, profiles and amounts of flexibility. For example, distal portion 109 is shown having a curved shape to demonstrate that at least a portion of elongate body 108 may be flexible. In various embodiments, elongate body 108 may have one or more of a round, ovoid, ellipsoid, flat, cambered flat, rectangular, square, triangular, symmetric or asymmetric cross-sectional shape. As, shown in FIGS. 3C and 3D, in the pictured embodiment, elongate body 108 has a relatively flat configuration, which may facilitate placement of body 108 between target and non-target tissues. Distal portion 109 of body 108 may be tapered, to facilitate its passage into or through narrow spaces as well as through small incisions on a patient's skin. Body 108 may also include a slightly widened portion around the area of window 111 and blades. In one embodiment, such as an embodiment used for modifying tissue in a spine, body 108 may have a small profile, such as having a height of not more than 10 mm at any point along its length and a width of not more than 20 mm at any point along its length, or more preferably a height not more than 5 mm at any point along its length and a width of not more than 10 mm at any point along its length, or even more preferably a height not more than 2 mm at any point along its length and a width of not more than 4 mm at any point along its length. Body 108 may be long enough to extend through a first incision on a patient, between target and non-target tissue, and out a second incision on a patient. Alternatively, body 108 may be long enough to extend through a first incision, between the target and non-target tissue, and to an anchoring location within the patient. In another alternative embodiment, body 108 may be long enough to extend through a first incision, between the target and non-target tissue, to a location nearby but distal to the target tissue within the patient, with some portion of tissue modification device 102 anchored to guide member 116. In some embodiments, elongate body 108 includes at least one feature for allowing passage of the body over a guidewire or other guide member or to allow passage of one or more guide members over or through body 108. For example, in various embodiments body 108 may include one or more guidewire lumens, rails, tracks, lengthwise impressions or some combination thereof.

In one embodiment, elongate body 108 is predominantly flexible along its length and comprises any suitable flexible material, such as thin, flexible metals, plastics, fabrics or the like. In some embodiments, it may be advantageous to include one or more rigid sections in elongate body 108, such as to impart pushability to a portion of body 108 or to facilitate application of force to tissue modification members 110 without causing unwanted bending or kinking of elongate body 108. In such embodiments, rigidity may be conferred by using additional materials in body 108 or by making the rigid portions thicker or wider or of a different shape.

Handle 104 may have any suitable configuration according to various embodiments. Similarly, actuator 106 may include any of a number of actuation devices in various embodiments. In the embodiment shown in FIG. 3A, actuator 106 comprises a trigger or moving handle portion, which is grasped by a user and pulled or squeezed toward handle 104 to bring blades 110 together to cut tissue. In an alternative embodiment, actuator 106 instead may include a switch or button for activating a radiofrequency surgical ablation tissue modifying member. In yet another embodiment, actuator 106 may include a combination trigger and switch, one or more pull wires, any suitable form of lever and/or some combination thereof.

FIGS. 3B-3D show in greater detail a portion of tissue modification device 102. In these figures, window 111 and blades 110 are more clearly seen. In one embodiment, at least a portion of elongate body 108 and blades 110 may have a slightly curved configuration. In alternative embodiments, at least a portion of elongate body 108 and blades 110 may be flat. In other alternative embodiments, tissue modification members such as blades 110 may be proud to elongate body 108.

Blades 110 include a distal 110a and a proximal blade 110b that reside at the distal and proximal edges, respectively, of window 111 of elongate body 108. Window 111 of body 108 may accommodate both soft and hard tissue when the device is forcibly applied to the surface of a target tissue site. The top view of the distal portion of elongate body 108, shown in FIG. 3C, depicts the angled edges of distal blade 110a and proximal blade 110b, which facilitate shearing of target tissue. In alternative embodiments, blades 110 may have any of a number of alternative shapes and configurations. The distal portion of body 108 may have a very low profile (height compared to width), as shown in side view FIG. 3D, where only blades 110 protrude from the top surface of the elongate body 108. In one embodiment, also as shown in FIG. 3D, a guidewire tube 120 (or lumen) may extend from (or be coupled with) a lower surface of elongate body 108. The lower surface of elongate body 108 is an example of a protective or non-tissue-modifying surface.

In one embodiment, distal blade 110a is coupled with two pull-wires 118, as seen in FIGS. 3C, 3E and 3F. Pull-wires 118 coupled to and translated by actuator 106 on handle 104 may be used to drive distal blade 110a proximally to contact the cutting edge of proximal blade 110b, thus cutting tissue. Other alternative mechanisms for driving blades 110, such as gears, ribbons or belts, magnets, electrically powered, shape memory alloy, electro magnetic solenoids and/or the like, coupled to suitable actuators, may be used in alternative embodiments. As mentioned, in one embodiment distal blade 110a and/or proximal blade 110b may have an outwardly curvilinear shape along its cutting edge. Alternatively, distal blade 110a may have a different blade shape, including flat, rectilinear, v-shaped, and inwardly curvilinear (concave vs. convex). The cutting edge of either blade 110 may have a sharp edge formed by a simple bevel or chamfer. Alternatively or in addition, a cutting edge may have tooth-like elements that interlock with a cutting edge of an opposing blade, or may have corrugated ridges, serrations, rasp-like features, or the like. In various embodiments, both blades 110 may be of equal sharpness, or alternatively one blade 110 may be sharp and the other substantially flat to provide a surface against which the sharp blade 110 may cut. Alternately or in addition, both cutting edges may be equally hard, or a first cutting edge may be harder than a second, the latter of which deflects under force from the first harder edge to facilitate shearing of the target tissue.

FIGS. 3E and 3F show cross-sectional views through elongate body at lines A-A and B-B, respectively, of FIG. 3C. In some embodiments, all or a portion of elongate body 108, such as the lower surface shown in FIG. 3E, may include a lubricious surface for facilitating manipulation of the tool in the surgical space and at the anatomical site. The lubricious lower surface also provides a barrier between blades 110 and non-target tissue in the surgical space. The lower surface may include a guide member lumen 120 to accommodate a guidewire or other access device or rail. FIG. 3E shows distal blade 110 coupled with pull wires 118. FIG. 3F shows proximal blade 110b, which is not coupled with pull wires 118 but rather fixed to body 108. In various alternative embodiments, proximal blade 110b may be movable distally while distal blade 110a is static, both blades may be moved toward one another, or a different number of blades may be used, such as one blade drawn toward a backstop or more than two blades, one or more of which may be mobile. In various alternative embodiments, guide member lumen 120 may be accommodated on a side surface or more centrally within elongate body 108. In further alternative embodiments, the one or more guide member lumens 120 may comprise one or more various cross sectional shapes, for example substantially round, substantially oval, or substantially rectabular, to accommodate alternative guide members, for example flat or rectangular guidewires, needles or rails. In still other alternative embodiments guide member lumen 120 may be adjustably coupled with the elongate body 108 to enable manipulation of the location of the elongate body 108 and therefore the tissue modifying members 110 relative to the guiding member.

Referring now to FIGS. 3G-3I, blades 110 are shown in their closed position. In one embodiment, when distal blade 110a is drawn proximally to cut tissue, at least some of the cut tissue is captured in a hollow interior portion of elongate body 108. Various embodiments may further include a cover, a cut tissue housing portion and/or the like for collecting cut tissue and/or other tissue debris. Such collected tissue and debris may then be removed from the patient during or after a tissue modification procedure. During a given tissue modification procedure, distal blade 110a may be drawn proximally to cut tissue, allowed to retract distally, and drawn proximally again to further cut tissue as many times as desired to achieve a desired amount of tissue cutting.

Blades 110 may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the blades or for portions or coatings of the blades may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades 110 may be manufactured using metal injection molding (MIM), CNC machining, injection molding, grinding and/or the like. Pull wires 118 be made from metal or polymer and may have circular, oval, rectangular, square or braided cross-sections. In some embodiments, a diameter of a pull wire 118 may range from about 0.001″-0.050″, and more preferably from about 0.010″-0.020″.

Depending on the tissue to be treated or modified, activating blades 110 (or other tissue modifying members in alternative embodiments) may cause them to modify target tissue along an area having any of a number of suitable lengths. In use, it may also be advantageous to limit the extent of action of blades 110 or other tissue modifying members to a desired length of tissue, thus not allowing blades 110 to affect tissue beyond that length. In so limiting the effect of blades, unwanted modification of, or damage to, surrounding tissues and structures may be limited or even eliminated. In one embodiment, for example, where the tissue modification device is used to modify tissue in a spine, blades 110 may operate along a length of target tissue of no more than 10 cm, and preferably no more than 6 cm, and even more preferably no more than 3 cm. Of course, in other parts of the body and to address other tissues, different tissue modification devices may be used and tissue modifying members may have many different lengths of activity. In one embodiment, to facilitate proper location of tissue modifying members, such as blades 110, relative to target tissue, the tissue modifying members and/or the elongate body and/or one or more additional features intended for just such a purpose may be composed of a material readily identifiable via x-ray, fluoroscopic, magnetic resonance or ultrasound imaging techniques.

In various embodiments, a number of different techniques may be used to prevent blades 110 (or other tissue modifying members) from extending significantly beyond the target tissue. In one embodiment, for example, preventing blades 110 from extending significantly beyond the target tissue involves holding tissue modification device 102 as a whole predominantly stable to prevent device 102 from translating in a direction toward its proximal portion or toward its distal portion while activating blades 110. Holding device 102 stable is achieved by anchoring one end of the device and applying tensioning force at or near the other end, as described further below.

In the embodiment shown in FIGS. 3A-3I, pull wires 118 are retracted proximally by squeezing actuator 106 proximally. In an alternative embodiment, squeezing actuator 106 may cause both blades 110 to translate inward so that they meet approximately in the middle of window 111. In a further embodiment, distal blade 110a may be returned to it's starting position by a pulling force generated from the distal end of device 102, for example by using a distal actuator that is attached to distal wires, or by pulling on the distal guide member which is attached to distal blade 110a. In yet another alternative embodiment, proximal blade 110b may be moved to cut by a pulling force generated from the distal end of device 102, for example by using a distal actuator that is attached to distal wires, or by pulling on the distal guide member which is attached to proximal blade 110b. In yet another embodiment, squeezing actuator 106 may cause proximal blade 110b to move distally while distal blade 110a stays fixed. In other alternative embodiments, one or more blades 110 may move side-to-side, one or more blades 110 may pop, slide or bow up out of window 111 when activated, or one or more blades 110 may expand through window. In another embodiment, one or more blades 110 and/or other tissue modifying members of device 102 may be powered devices configured to cut, shave, grind, abrade and/or resect target tissue. In other embodiments, one or more blades may be coupled with an energy transmission device, such as a radiofrequency (RF) or thermal resistive device, to provide energy to blade(s) 110 for cutting, ablating, shrinking, dissecting, coagulating or heating and thus enhancing tissue modification. In another embodiment, a rasp or file may be used in conjunction with or coupled with one or more blades. In any of these embodiments, use of actuator 106 and one or more moving blades 110 provides for tissue modification with relatively little overall translation or other movement of tissue modification device 102. Thus, target tissue may be modified without extending blades 110 or other tissue modification members significantly beyond an area of target tissue to be treated.

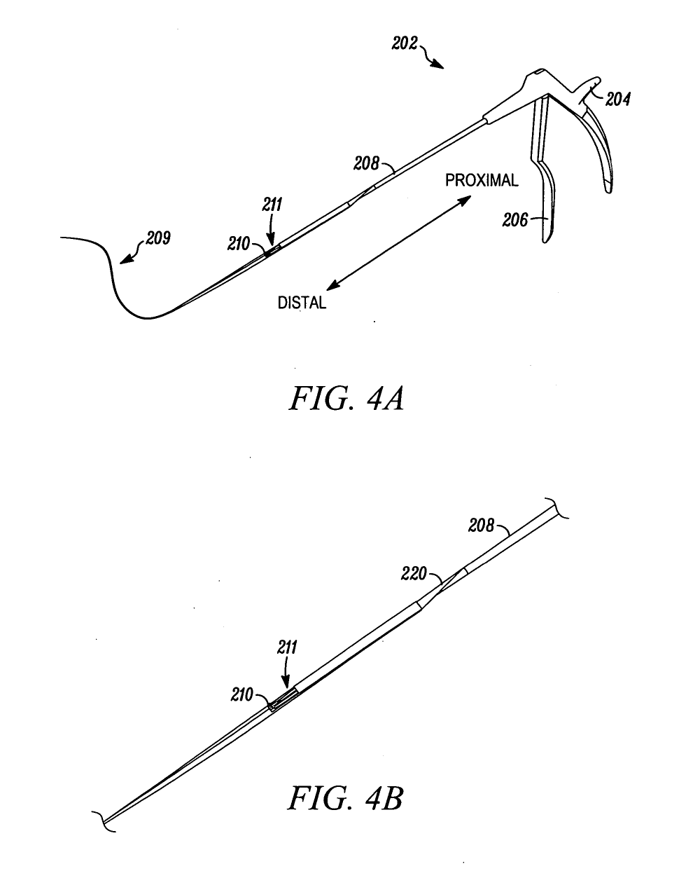

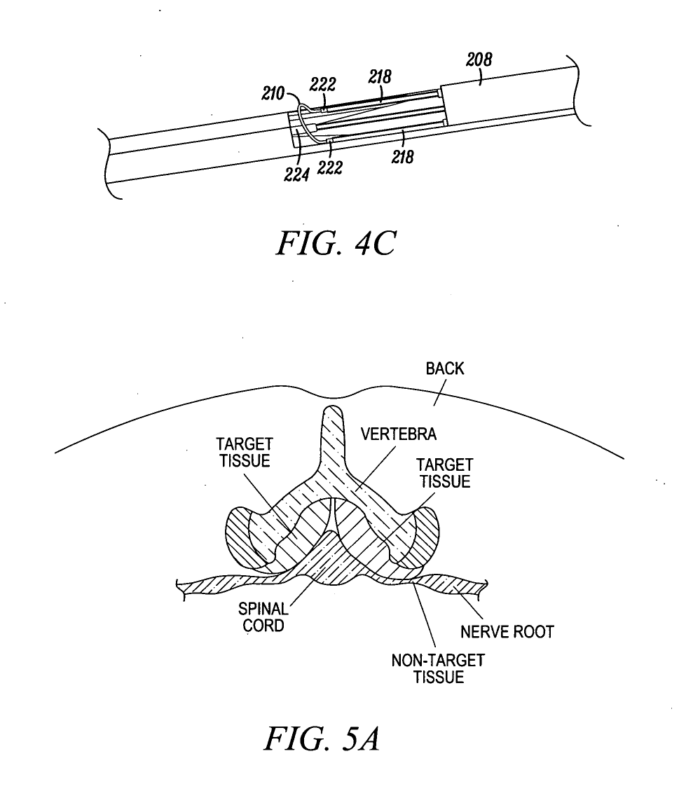

Referring now to FIGS. 4A-4C, in an alternative embodiment, a tissue modification device 202 may include an elongate body 208 having a proximal portion and a distal portion 209, a handle 204 and actuator 206 coupled with proximal portion, and a window 211 and tissue modifying member 210 disposed near distal portion 209. As seen more clearly in FIGS. 4B and 4C, in the embodiment shown, tissue modifying member 210 comprises an RF electrode wire loop. Wire loop 210 may comprise any suitable RF electrode, such as those commonly used and known in the electrosurgical arts, and may be powered by an internal or external RF generator, such as the RF generators provided by Gyrus Medical, Inc. (Maple Grove, Minn.). Any of a number of different ranges of radio frequency may be used, according to various embodiments. For example, some embodiments may use RF energy in a range of between about 70 hertz and about 5 megahertz. In some embodiments, the power range for RF energy may be between about 0.5 Watts and about 200 Watts. Additionally, in various embodiments, RF current may be delivered directly into conductive tissue or may be delivered to a conductive medium, such as saline or Lactate Ringers solution, which may in some embodiments be heated or vaporized or converted to plasma that in turn modifies target tissue. Distal portion 209 includes a tapered tip, similar to that described above, to facilitate passage of elongate body 208 into narrow anatomical sites. Handle 204 and actuator 206 are similar to those described above, although in the embodiment of FIGS. 4A-4C, actuator 206 may be used to change the diameter of the wire loop 210. Using actuator 206, wire loop 210 may be caused to extend out of window 211, expand, retract, translate and/or the like. Some embodiments may optionally include a second actuator (not shown), such as a foot switch for activating an RF generator to delivery RF current to an electrode.

Elongate body 208 may be fabricated from any suitable material and have any of a number of configurations. In one embodiment, body 208 comprises a metal tube with a full-thickness slit (to unfold the tube into a flat form—not shown) or stiffening element (not shown). The split tube provides for a simple manufacturing process as well as a conductive pathway for bi-polar RF operation.

Referring to FIG. 4C, insulators 222 may be disposed around a portion of wire loop 210 so that only a desired portion of wire loop 210 may transfer RF current into the tissue for tissue modifying capability. Wire loop 210, covered with insulators 222 may extend proximally into support tubes 218. In various alternative embodiments, an electrode tissue modifying member (of which wire loop 210 is but one example) may be bipolar or monopolar. For example, as shown in FIG. 4C, a sleeve 224 housed toward the distal portion of window 211 may act as a return electrode for wire loop 210 in a bipolar device. Wire loop electrodes 210 may be made from various conductive metals such as stainless steel alloys, nickel titanium alloys, titanium alloys, tungsten alloys and the like. Insulators 222 may be made from a thermally and electrically stable polymer, such as polyimide, polyetheretherketone (PEEK), polytetrafluoroethylene (PTFE), polyamide-imide, or the like, and may optionally be fiber reinforced or contain a braid for additional stiffness and strength. In alternative embodiments, insulators 222 may be composed of a ceramic-based material.

In one embodiment, wire loop 210 may be housed within elongate body 208 during delivery of tissue modification device 202 into a patient, and then caused to extend up out of window 211, relative to the rest of body 208, to remove tissue. Wire loop 210 may also be flexible so that it may pop or bow up out of window 211 and may deflect when it encounters hard tissue surfaces. Wire loop 210 may have any of a number of shapes, such as curved, flat, spiral or ridged. Wire loop 210 may have a diameter similar to the width of body 208, while in alternative embodiments it may expand when extended out of window 211 to have a smaller or larger diameter than that of body 208. Pull wires (not shown) may be retracted proximally, in a manner similar to that described above, in order to collapse wire loop 210, decrease the diameter and lower the profile of the wire loop 210, and/or pull wire loop 210 proximally to remove tissue or be housed within body 208. The low profile of the collapsed wire loop 210, facilitates insertion and removal of tissue modification device 202 prior to and after tissue modification. As the wire loop 210 diameter is reduced, support tubes 218 deflect toward the center of elongate body 208.

In an alternative embodiment (not shown), tissue modification device 202 may include multiple RF wire loops 210 or other RF members. In another embodiment, device 202 may include one or more blades as well as RF wire loop 210. In such an embodiment, wire loop 210 may be used to remove or otherwise modify soft tissues, such as ligamentum flavum, or to provide hemostasis, and blades may be used to modify hard tissues, such as bone. In other embodiments, as described further below, two separate tissue modification devices (or more than two devices) may be used in one procedure to modify different types of tissue, enhance modification of one type of tissue or the like.

In other alternative embodiments, tissue modification devices 202 may include tissue modifying members such as a rongeur, a curette, a scalpel, a scissors, a forceps, a probe, a rasp, a file, an abrasive element, one or more small planes, a rotary powered mechanical shaver, a reciprocating powered mechanical shaver, a powered mechanical burr, a laser, an ultrasound crystal a cryogenic probe, a pressurized water jet, a drug dispensing element, a needle, a needle electrode, or some combination thereof. In some embodiments, for example, it may be advantageous to have one or more tissue modifying members that stabilize target tissue, such as by grasping the tissue or using tissue restraints such as barbs, hooks, compressive members or the like. In one embodiment, soft tissue may be stabilized by applying a contained, low-temperature substance (for example, in the cryo-range of temperatures) that hardens the tissue, thus facilitating resection of the tissue by a blade, rasp or other device. In another embodiment, one or more stiffening substances or members may be applied to tissue, such as bioabsorbable rods.

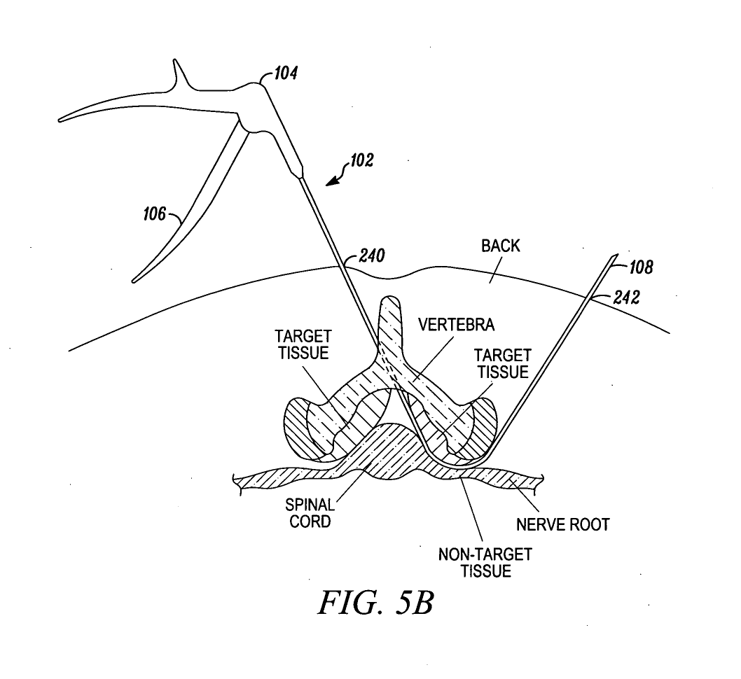

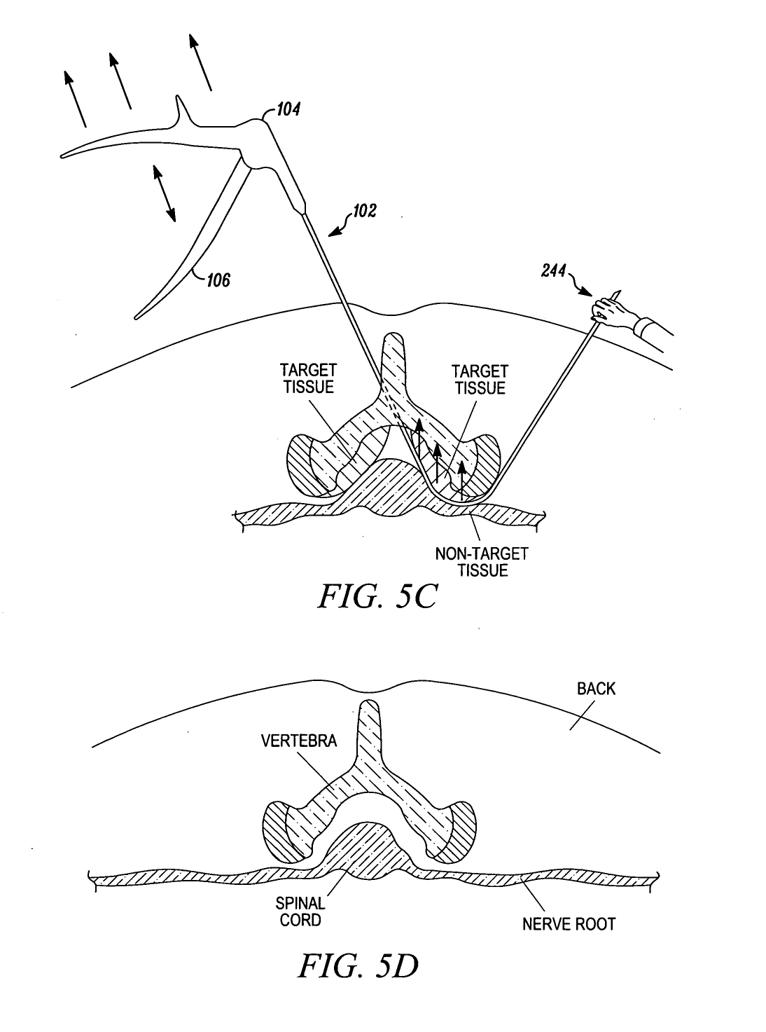

Referring now to FIGS. 5A-5D, one embodiment of a method for modifying tissue in a spine is demonstrated in simplified, diagrammatic, cross-sectional views of a portion of a patient's back and spine. FIG. 5A shows a portion of the patient's back in cross section, with a portion of a vertebra, the spinal cord with branching nerve roots, and target tissue, which in this illustration is the ligamentum flavum and possibly a portion of the facet capsule. The target tissue is typically impinging directly on one or more of the group including nerve roots, neurovascular structures, dorsal root ganglia, cauda equina, or individual nerves.

In FIG. 5B, tissue modification device 102 has been positioned in the patient's back to perform a tissue modification procedure. Various methods, devices and systems for introducing device 102 into the patient and advancing it to the position for modifying tissue are described in further detail below. Generally, device 102 may be positioned via a percutaneous or open surgical procedure, according to various embodiments. In one embodiment, device 102 may be inserted into the patient through a first incision 240, advanced into the spine and between target tissue and non-target tissue (such as spinal cord, nerve roots, nerves and/or neurovascular tissue), and further advanced so a distal portion of elongate body 108 exits a second (or distal) incision 242 to reside outside the patient. In positioning device 102, one or more tissue modifying members (not shown) are positioned to face the target tissue, while one or more protective portions of elongate body 108 face non-target tissue.

Referring to FIG. 5C, once device 102 is positioned in a desired location, anchoring force may be applied at or near the distal portion of elongate body 108. In one embodiment, applying anchoring force involves a user 244 grasping body 108 at or near its distal portion. In alternative embodiments, as described further below, anchoring force may be applied by deploying one or more anchor members disposed at or near the distal portion of body 108, or by grasping a guidewire or other guide member extending through at least part of body 108. Once the anchoring force is applied, proximally-directed tensioning force may be applied to device 102, such as by pulling proximally on handle 104 (one-directional, diagonal arrows). This tensioning force, when applied to the substantially anchored device 102, may help urge the tissue modifying member(s) against the target tissue (one-directional, vertical arrows near target tissue), thus enhancing contact with the target tissue and facilitating its modification. With the tissue modifying member(s) contacting the target tissue, actuator 106 may be squeezed or pulled (two-headed arrow) to cause the tissue modifying member(s) to modify tissue. (Alternative actuators may be activated in different ways in alternative embodiments.)

In various alternative embodiments, certain of the above-described steps may be carried out in different order. For example, in one embodiment the distal portion of elongate body 108 may be anchored within or outside the patient before the tissue modifying members are positioned adjacent the target tissue. In another alternative embodiment, the proximal portion of device 102 may be anchored, and the tensioning force may be applied to the distal portion of device 102. In yet another embodiment, tensioning force may be applied to both ends of the device. In yet another embodiment, a second handle and actuator may be coupled with the distal end of body 108 after it exits the patient's back, allowing tensioning forces as well as tissue modifying actuation to occur at both the proximal and distal portions of device 102. By anchoring one end of device 102 and applying tensioning force to the opposite end, contact of the tissue modifying members with the target tissue is enhanced, thus reducing or eliminating the need for translating or otherwise moving device 102 as a whole and reducing the overall profile and the resulting access pathway required to position the device. Reducing movement and profile of device 102 and using tissue modifying members confined to a relatively small area of device 102 helps facilitate target tissue modification while minimizing or eliminating damage to surrounding tissues or structures.

As mentioned above, tissue may be modified using one tissue modification device or multiple devices, according to various embodiments. In one embodiment, for example, an RF electrosurgical tissue modification device may be used in the patient to remove soft tissue such as ligament, and a bladed tissue modification device such as a rongeur may then be used to remove additional soft tissue, calcified soft tissue, or hard tissue such as bone. In some embodiments, such multiple devices may be inserted, used and removed serially, while in alternative embodiments such devices may be inserted into the patient at the same time to be used in combination.

Referring to FIG. 5D, using one or more tissue modification devices 102, a desired amount of target tissue may be removed from more than one area in the spine. FIGS. 5A-5C demonstrate removal of target tissue on one side of the spine, and that method or a similar method may also be used to remove target tissue on an opposite side of the spine, as shown in FIG. 5D, where target tissue has been removed from both sides. That the desired amount of tissue has been removed may be confirmed by tactile feedback from the device or from a separate device, by testing nerve conduction through one or more previously impinged nerves, by testing blood flow through one or more previously impinged blood vessels, by passing (independently or over the guide member) a measurement probe or sound through the treated portion, through one or more radiographic tests, through some combination thereof, or by any other reasonable means.

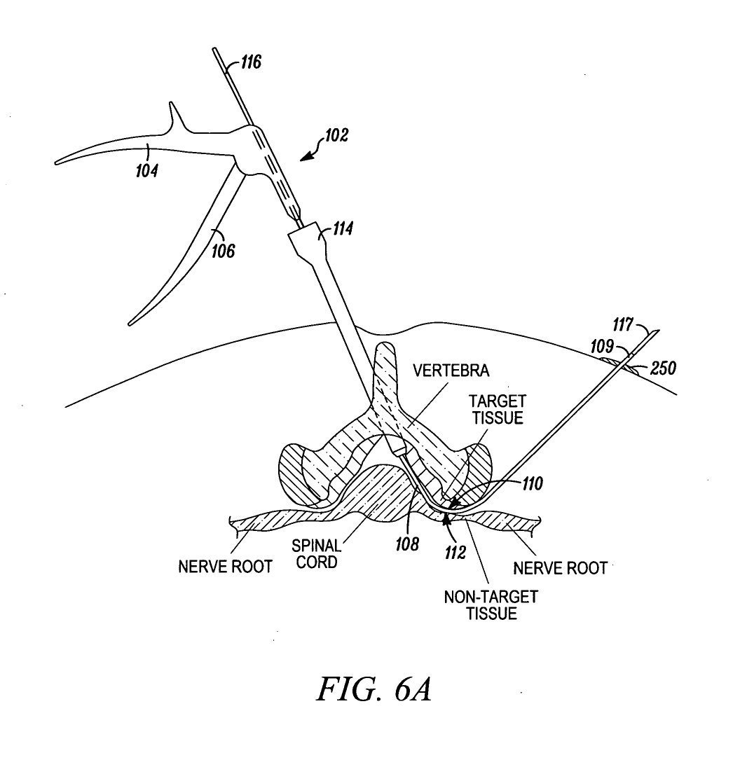

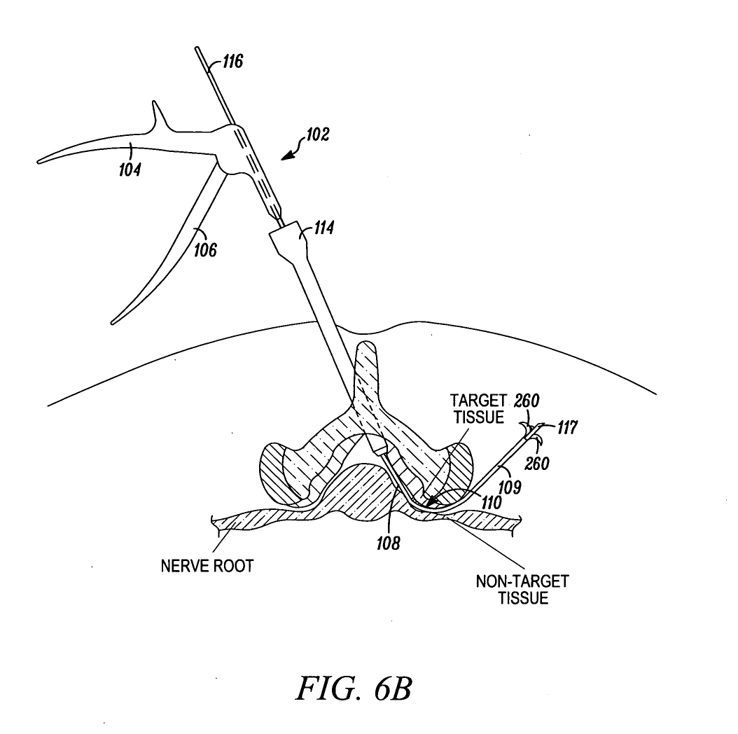

Referring now to FIG. 6A, tissue modification device 102 is shown with one embodiment of a distal anchoring member 250 deployed at the patient's skin. In various embodiments, anchoring members may include but are not limited to one or more handles, barbs, hooks, screws, toggle bolts, needles, inflatable balloons, meshes, stents, wires, lassos, backstops or the like. In some embodiments, anchoring members 250 may be disposed at the extreme distal portion 109 of elongate body 108, while in other embodiments anchoring members 250 may be located more proximally. In the embodiment shown, anchoring members 250 are deployed at the patient's skin. In an alternative embodiment, anchoring may be achieved outside the patient by deploying one or more anchoring members 250 above the skin and having a user grasp the anchoring members 250. In an alternative embodiment, anchoring may be achieved outside the patient by deploying one or more anchoring members 250 above the skin and having a user grasp anchoring members 250, after tissue modification device 102 has been anchored to the guide member. In another alternative embodiment, anchoring may be achieved outside the patient by attaching anchoring member 250 to an external device, for example one that is mounted on the patient or on the procedure table. In a further alternative embodiment, anchoring may be achieved outside the patient by attaching the guide member to an external device, for example one that is mounted to on the patient or on the procedure table, after tissue modification device 102 has been anchored to the guide member. Anchoring members 250 generally are deployable from a first, contracted configuration to facilitate delivery of device 102, to a second, expanded configuration to facilitate anchoring. This change in configuration may be achieved, for example, by using shape memory or super-elastic materials, by spring loading anchoring members 250 into body 108 or the like. In most embodiments, anchoring members 250 may also be collapsed down into the first, contracted configuration after a tissue modification procedure has been performed, to facilitate withdrawal of device 102 from the patient. In an alternative embodiment, anchoring members 250 may detach from body 108 and may be easily removable from the patient's skin.

FIG. 6B shows tissue modification device 102 with an alternative embodiment of a distal anchoring member 260. Here, distal anchoring member 260 includes multiple hooks or barbs extended out the distal portion 109 of elongate body 108 within the patient's back. In using such an embodiment, it may not be necessary to pass guide member 117 through a second, distal incision on the patient, although in some embodiments guide member 117 may extend significantly beyond distal portion 109. Anchoring member(s) 260, according to various embodiments, may be deployed so as to anchor to bone, ligament, tendon, capsule, cartilage, muscle, or any other suitable tissue of the patient. They may be deployed into vertebral bone or other suitable tissue immediately adjacent an intervertebral foramen or at a location more distant from the intervertebral foramen. When a tissue modification procedure is complete, anchoring members 260 are retracted within elongate body for removal of device 102 from the patient.

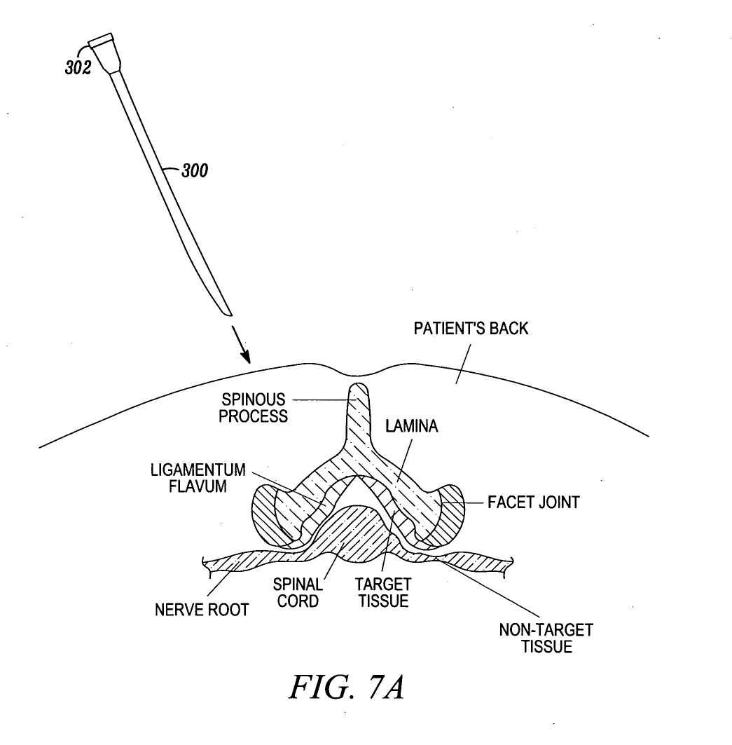

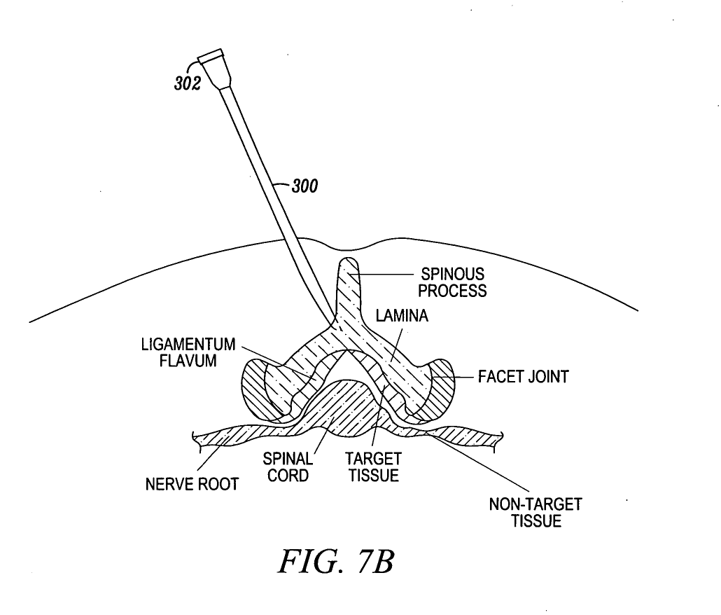

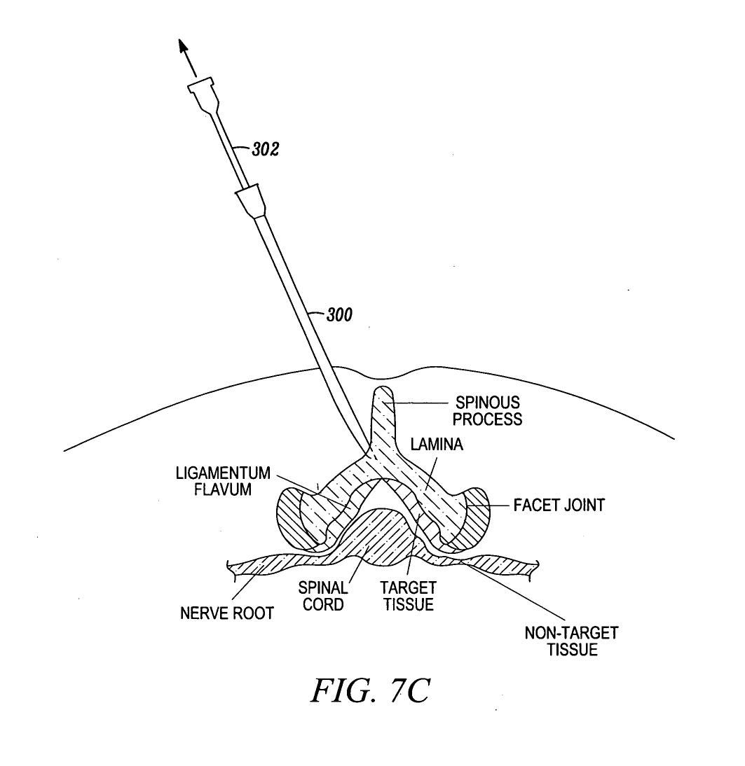

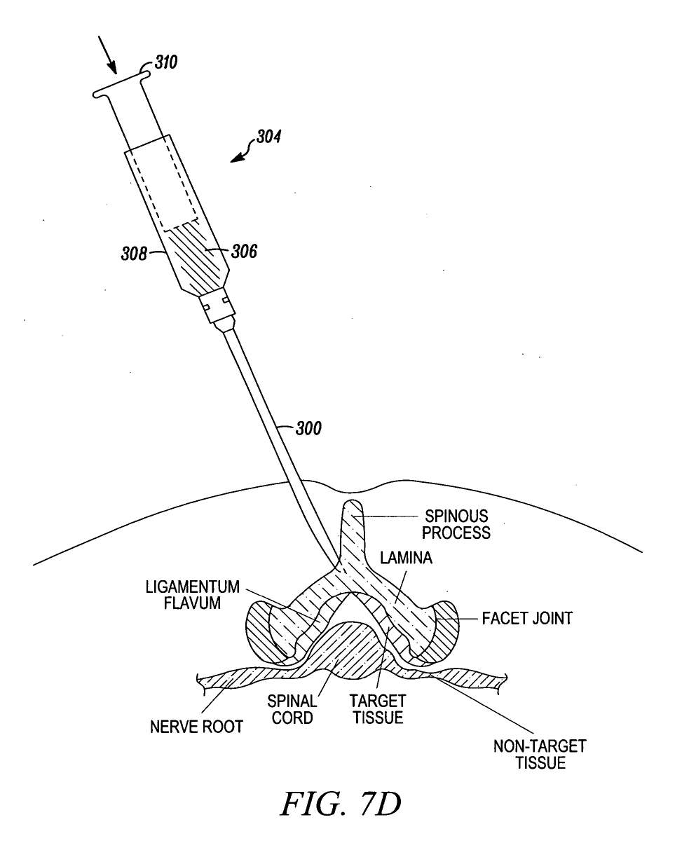

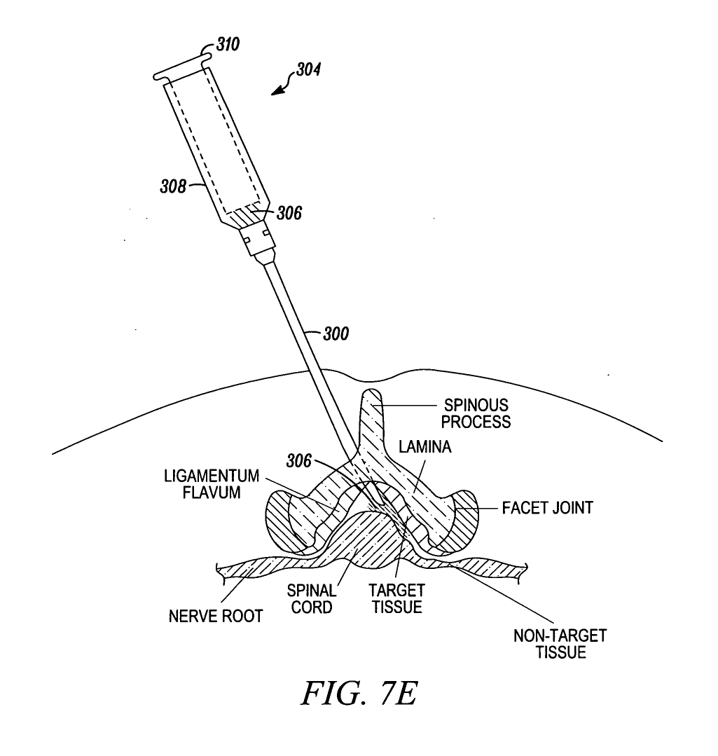

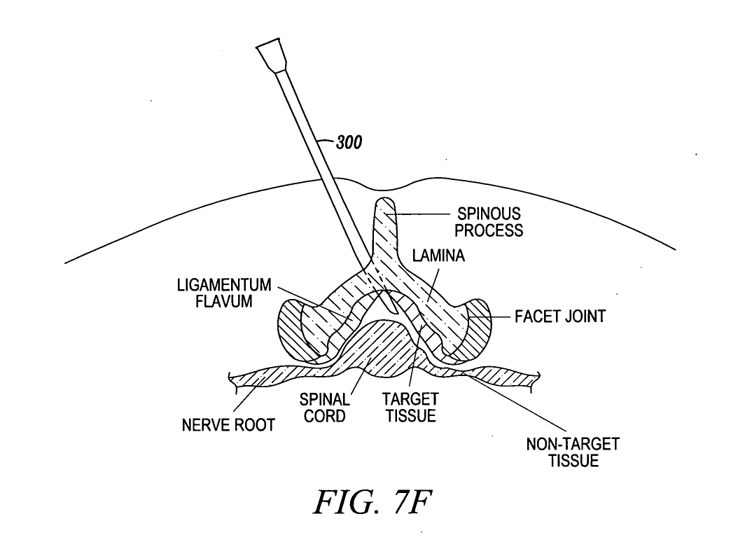

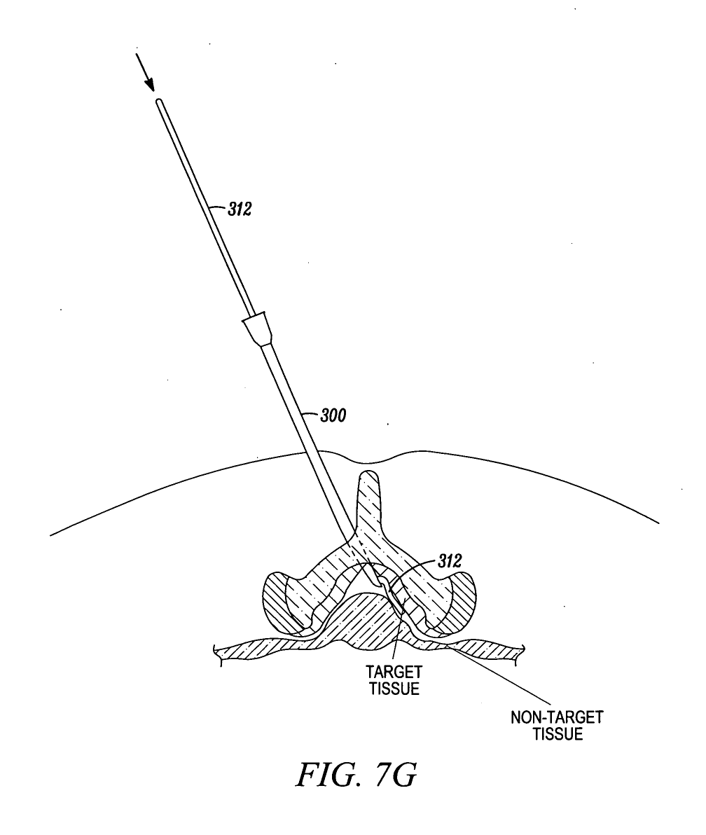

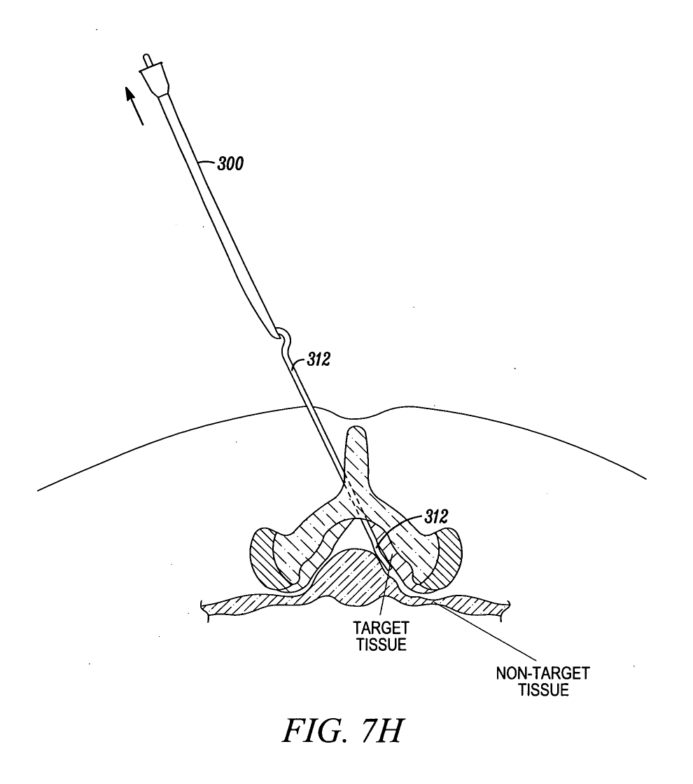

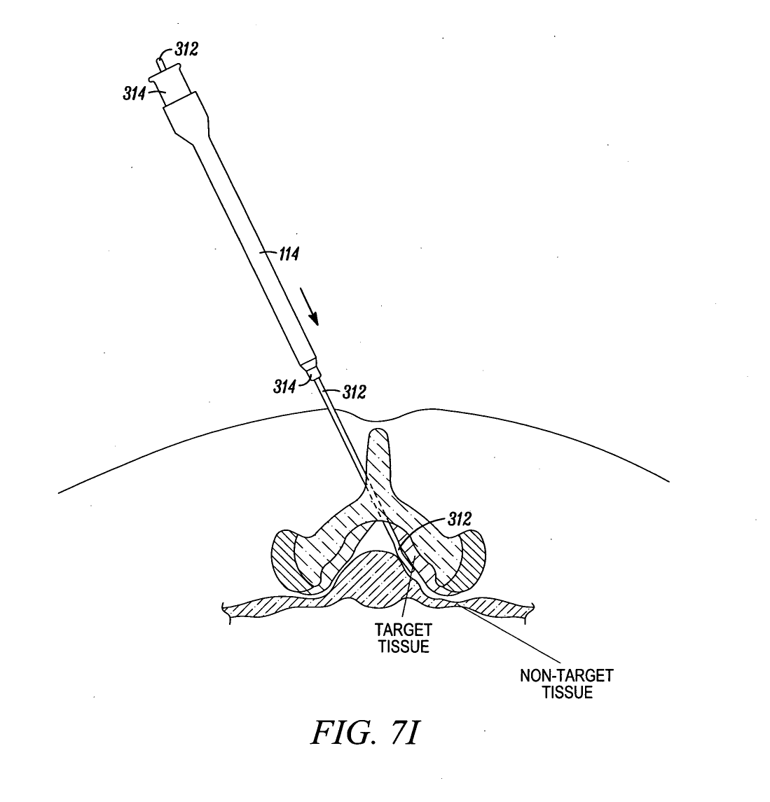

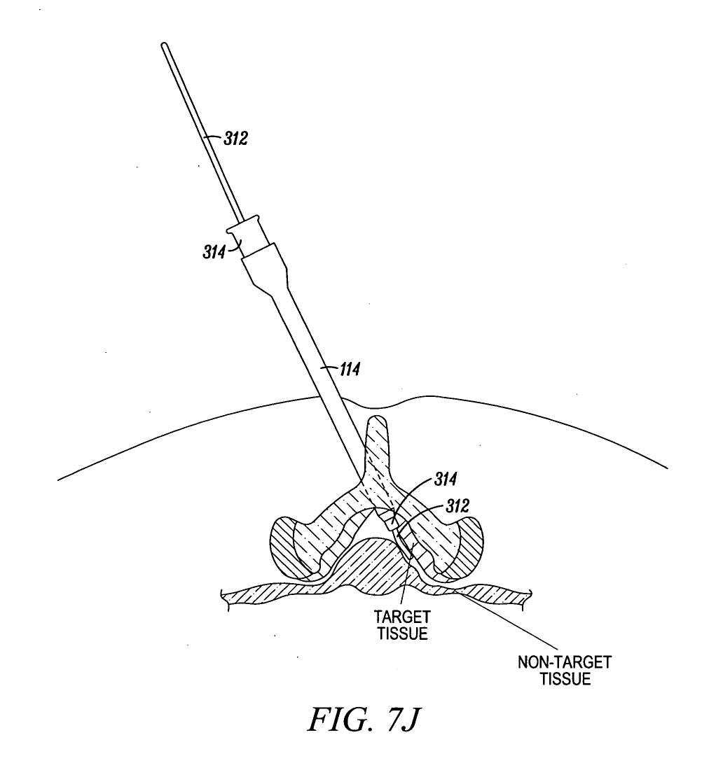

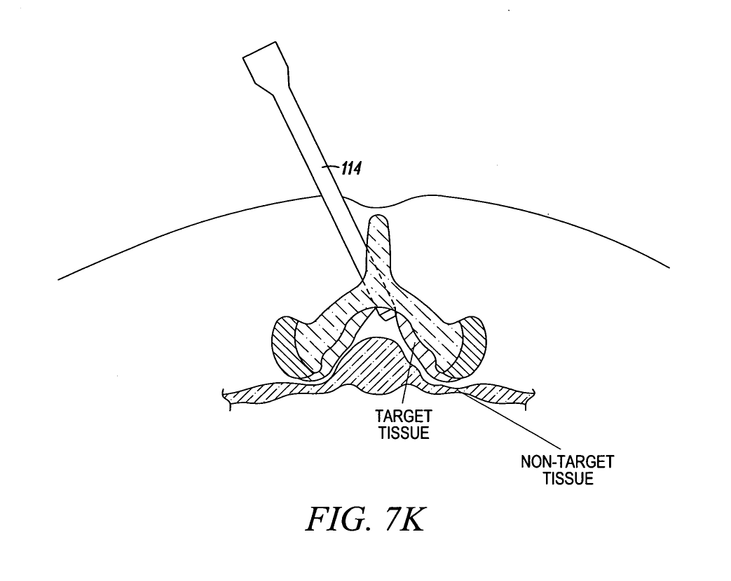

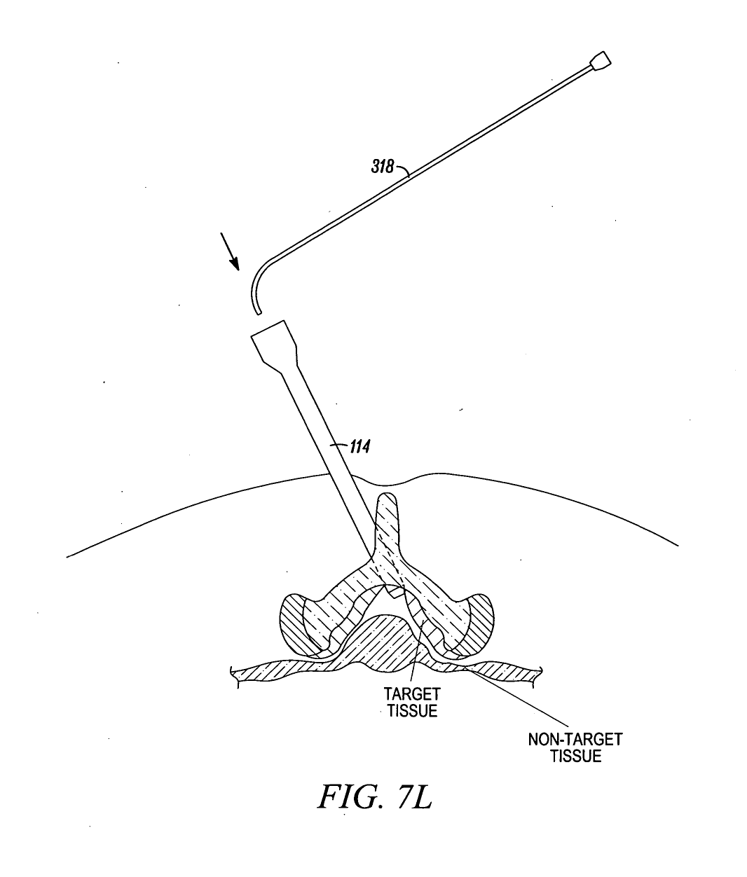

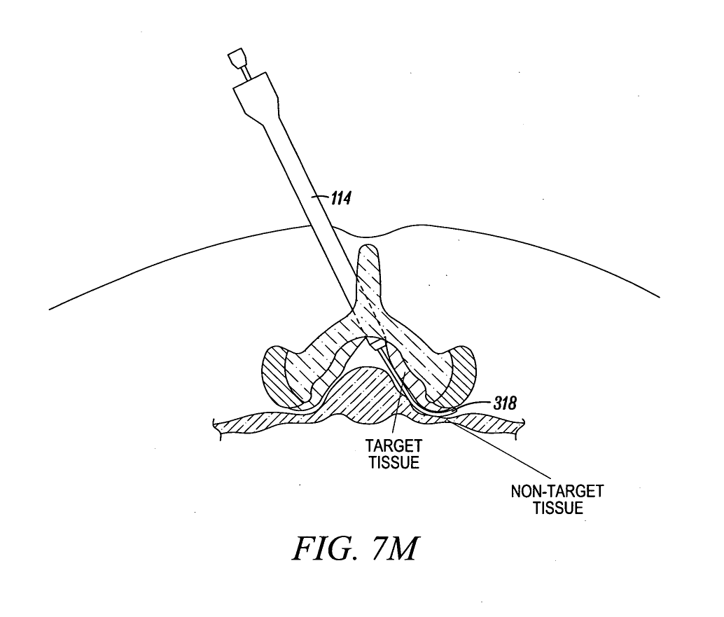

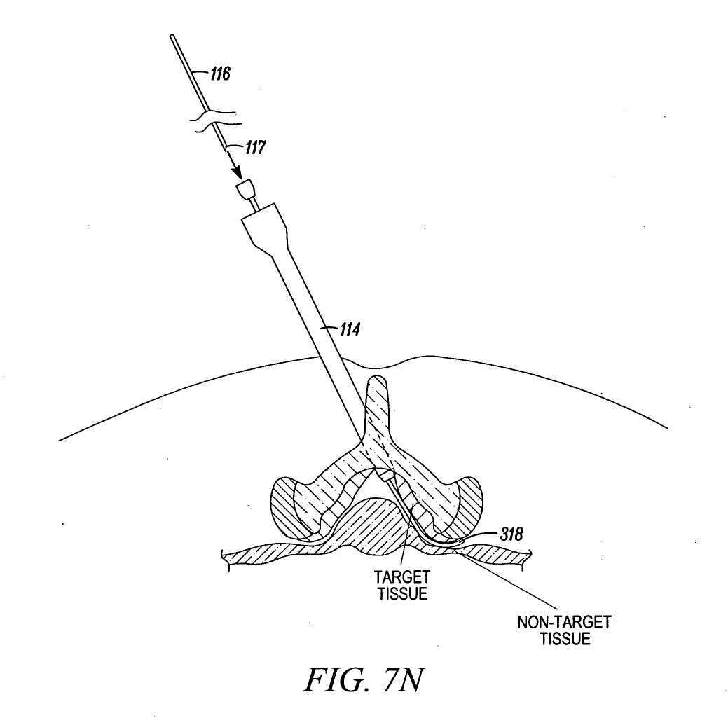

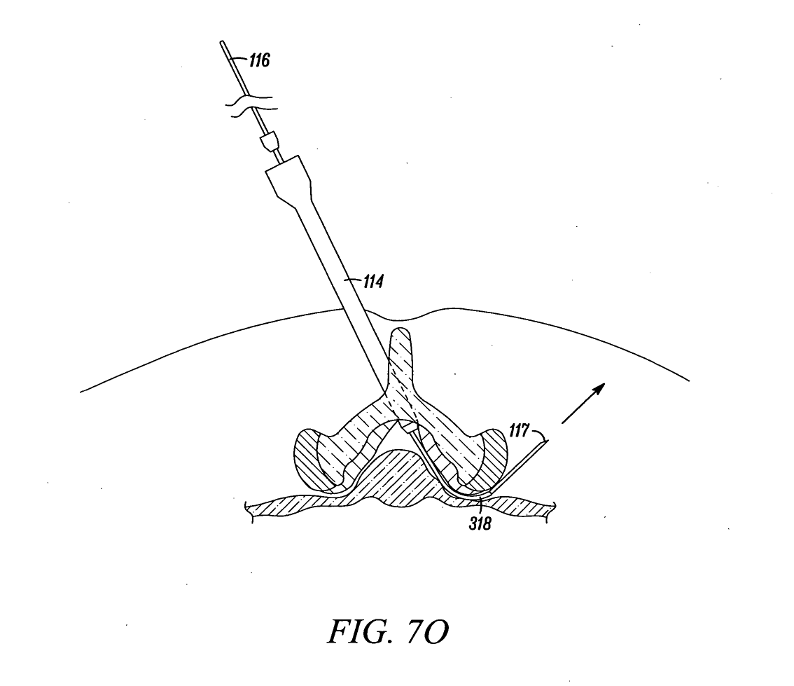

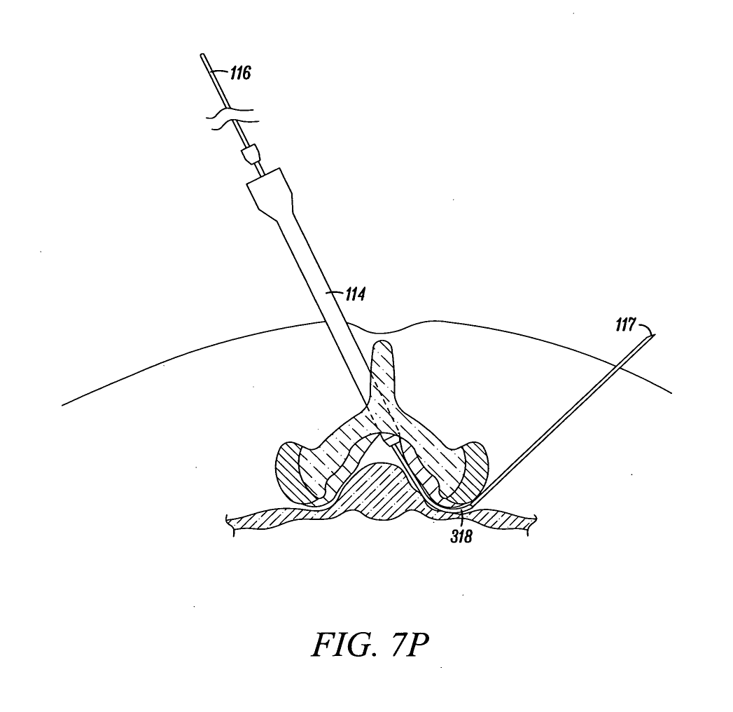

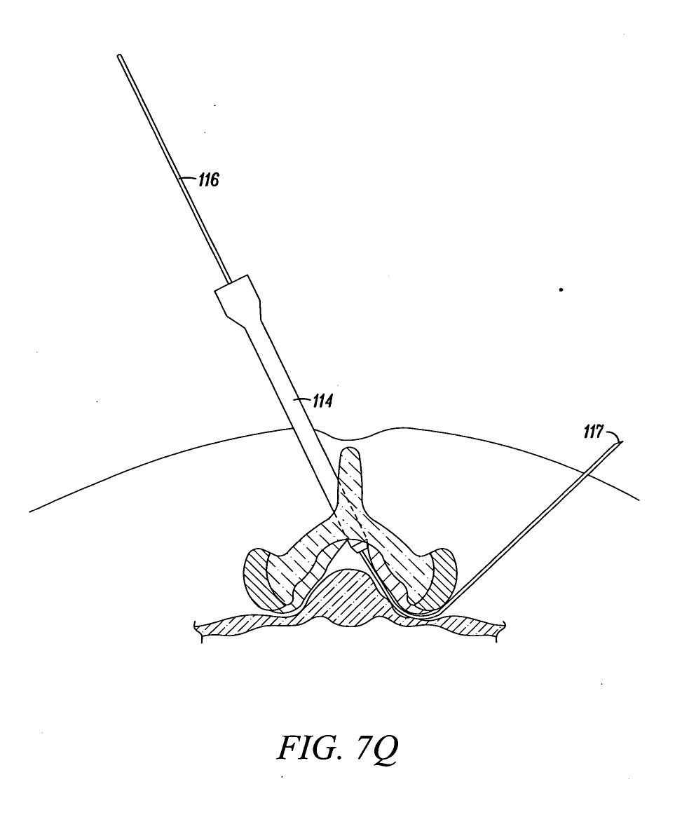

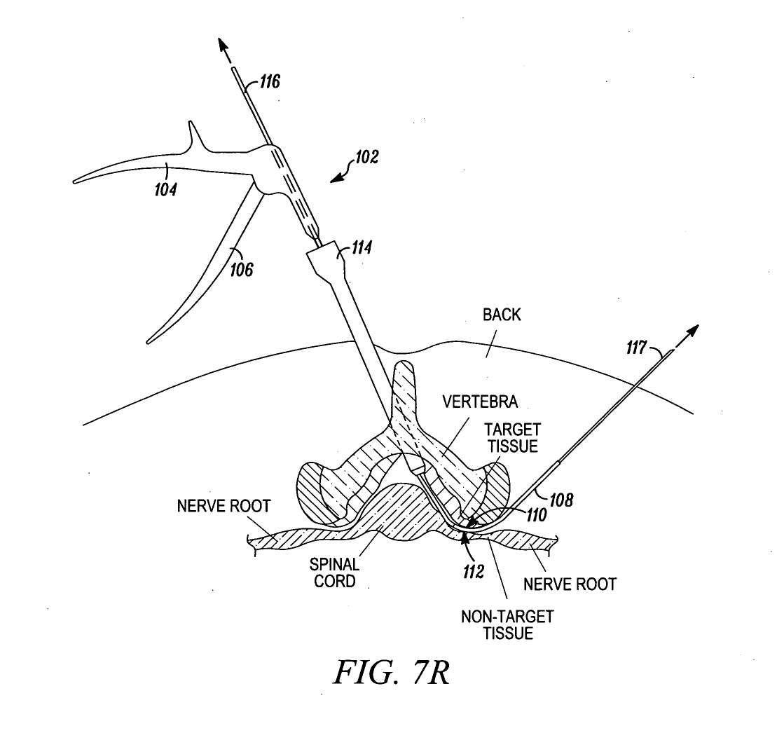

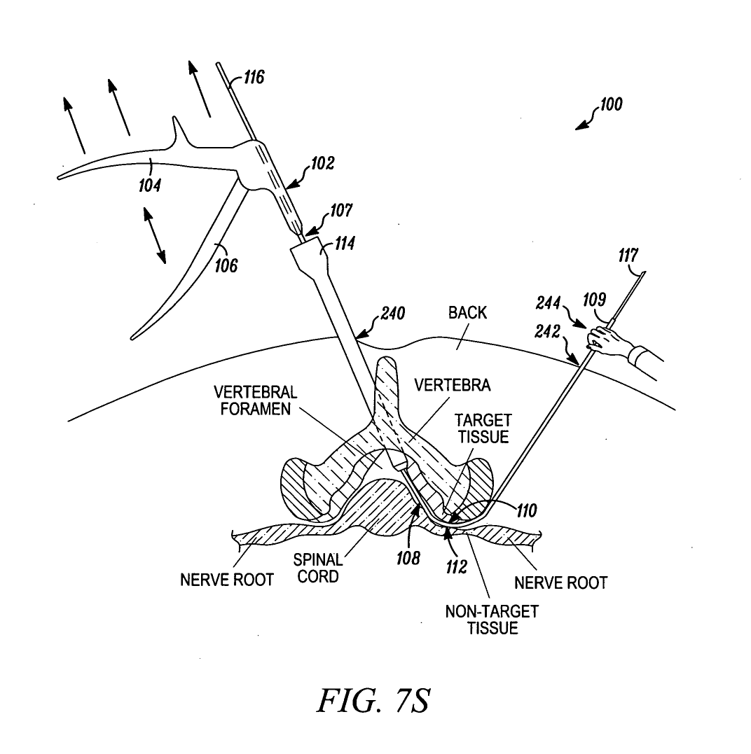

Referring now to FIGS. 7A-7S, a system and method for introducing a tissue modification device into a spine is demonstrated. This system and method may be referred to as an “access system” or “access method,” in that they provide or facilitate gaining access to a target tissue to be modified. Of course, the embodiment shown is merely one exemplary embodiment, and any of a number of other suitable methods, devices or systems may be used to introduce one or more devices for modifying tissue in spine. For example, in one alternative embodiment a spinal tissue modification procedure may be carried out through an open surgical approach. Therefore, the following description is provided primarily for exemplary purposes and should not be interpreted to limit the scope of the invention as it is defined in the claims.

Referring to FIG. 7A, in one embodiment a device delivery method first involves advancing an introducer cannula 300 coupled with a stylet 302 into the patient's back. Cannula 300 and stylet 302 are then passed between adjacent vertebrae and into the ligamentum flavum or an adjacent spinal ligament, as shown further in FIG. 7B. As shown in FIG. 7C, when the distal tip of cannula is positioned as desired, stylet 302 is removed. Referring to FIGS. 7D and 7E, a loss of resistance syringe 304 including a plunger 310, barrel 308 and fluid and/or air 306, is coupled with the proximal portion of cannula 300. The distal portion of cannula 300 is advanced through the ligamentum flavum until it enters the central spinal canal where a loss of resistance to pressure placed on plunger 310 is encountered, and fluid and/or air 306 is injected into central spinal canal to confirm correct placement of cannula 300 as shown in FIG. 7E. Syringe 304 is then removed, as in FIG. 7F, and a guidewire 312 with a non-rigid, atraumatic tip is advanced through cannula 300 into the central spinal canal, as in FIG. 7G. Next, cannula 300 is removed, as in FIG. 7H, leaving behind guidewire 312. As shown in FIGS. 7I and 7J, an introducer sheath 114, coupled with a dilator 314, is then advanced over guidewire 312 to position a distal portion of sheath 114 at a desired location within the spine. Dilator 314 and guidewire 312 are then removed, as in FIG. 7K.

Once introducer sheath 114 is in place, one or more curved or steerable guide devices 318 may be advanced through it to desired positions in and/or through the spine, as shown in FIGS. 7L and 7M. One or more guide members 116, may then be advanced through the guide device 318, as shown in FIGS. 7N-7P. Finally, guide device 318 may be removed, as in FIG. 7Q, and elongate body 108 of tissue modification device 102 may be advanced over guide member 116 and through introducer sheath 114 to a desired position in the spine, as in FIG. 7R. As shown in FIG. 7S, elongate body 108 may be tensioned to urge tissue modifying members 110 against target tissue, as shown with arrows at opposite ends of device 102, while distal portion 109 is anchored, in this case by hand 244. In an alternative embodiment, guide member 116 may be tensioned to urge tissue modifying members 110 against target tissue as shown in FIG. 7R.

Once tissue modification device 102 is in a desired position, tissues which may be modified in various embodiments include, but are not limited to, ligament, tendon, tumor, cyst, cartilage, scar, “bone spurs,” inflammatory and bone tissue. In some embodiments, modifying the target tissue reduces impingement of the tissue on a spinal cord, a branching nerve or nerve root, a dorsal root ganglia, and/or vascular tissue in the spine. Actuator 106 on handle 104 is activated to modify target tissue using tissue modification member(s) 110, while elongate body 108 is held relatively stable by hand 244 and by tension force applied to handle 104.

In various embodiments, the system and method described immediately above may include additional features or steps, may have fewer features or steps, may have an alternate order of implementation of steps, or may have different features or steps. For example, in some embodiments placement of device 102 will be performed in a medial-to-lateral direction (relative to the patient), while in alternative embodiments device placement will be performed lateral-to-medial. In some embodiments, one or more components of the system described may be anchored to the patient, such as guide member 116 or introducer sheath 114. In various embodiments, one or more guide members 116 may include one or more wires, rails or tracks and may be inserted through guide device 318, introducer sheath 114 without guide device 318, cannula 300, an epidural needle, a lumen of an endoscope, a lumen of a tissue shield or barrier device, a curved guide device 318 placed through a lumen of an endoscope, or the like. In other embodiments, for example, guide device 318 may be placed through introducer cannula 300 and then introducer sheath 114 may be passed over guide device 318. Tissue modification device 102 may similarly be inserted with or without using any of these devices or components in various combinations. Various guidewires 312, guide devices 318 and/or guide members 116 may be pre-shaped to have one or more curves, may be steerable, and/or may include one or more rails, tracks, grooves, lumens, slots, partial lumens, or some combination thereof.

In some embodiments, tissue modification device 102 is inserted through one or more hollow devices as described above (such as introducer sheath 114, as shown, or cannula 300 in an alternative embodiment) in such a way that device 102 expands upon extending out of a distal portion of the hollow delivery device thereby assuming a wider profile for modifying a greater amount of target tissue from a single location. In an alternative embodiment, device 102 retains the same overall profile during insertion and during use. In some embodiments, one or more delivery devices will remain in the patient during use of tissue modification device 102, while in alternative embodiments all delivery devices are removed from the patient when tissue modification device 102 is operating. In some embodiments, tissue modification device 102 may be slidably coupled with one or more delivery devices during delivery and/or during use. In one embodiment, tissue modification device 102 is advanced through introducer sheath 114 and sheath 114 is used as an irrigation and evacuation lumen to irrigate the area of the target tissue and evacuate removed tissue and other debris, typically by applying a vacuum. In alternative embodiments, tissue modification device 102 may include an irrigation and/or evacuation lumen to irrigate an area of the target tissue and evacuate removed tissue and other debris.

Some embodiments of an access system for facilitating tissue modification may further include one or more visualization devices (not shown). Such devices may be used to facilitate placement of the access system for introducing the tissue modification device, to facilitate tissue modification itself, or any combination of these functions. Examples of visualization devices that may be used include flexible, partially flexible, or rigid fiber optic scopes, rigid rod and lens endoscopes, CCD or CMOS chips at the distal portion of rigid or flexible probes, LED illumination, fibers or transmission of an external light source for illumination or the like. Such devices may be slidably couplable with one or more components of an access system or may be slidably or fixedly coupled with a tissue modification device. In other embodiments, additional or alternative devices for helping position, use or assess the effect of a tissue modification device may be included. Examples of other such devices may include one or more neural stimulation electrodes with EMG or SSEP monitoring, ultrasound imaging transducers external or internal to the patient, a computed tomography (CT) scanner, a magnetic resonance imaging (MRI) scanner, a reflectance spectrophotometry device, and a tissue impedance monitor disposed across a bipolar electrode tissue modification member or disposed elsewhere on a tissue modification device or disposed on the access system.

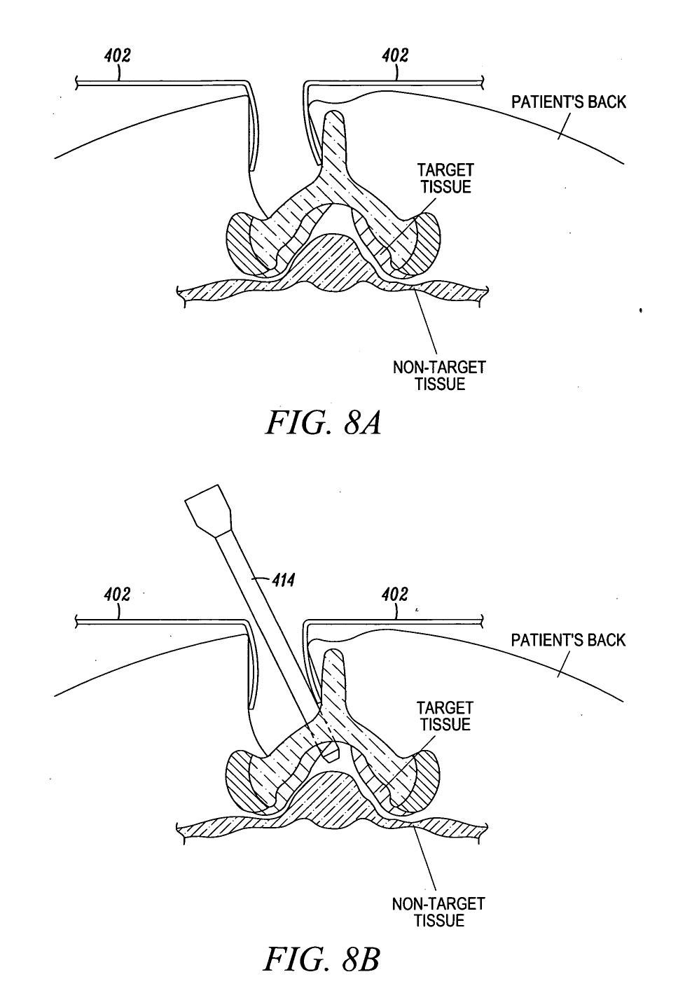

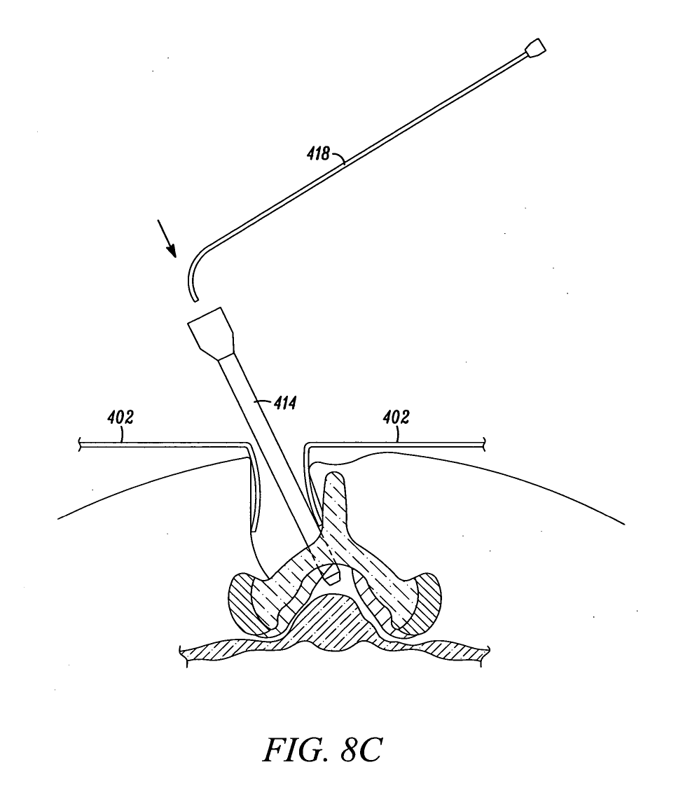

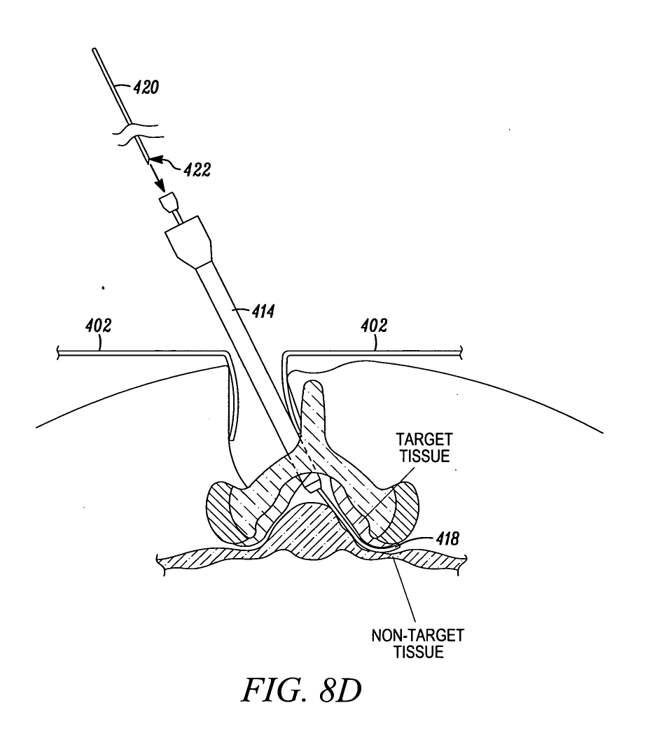

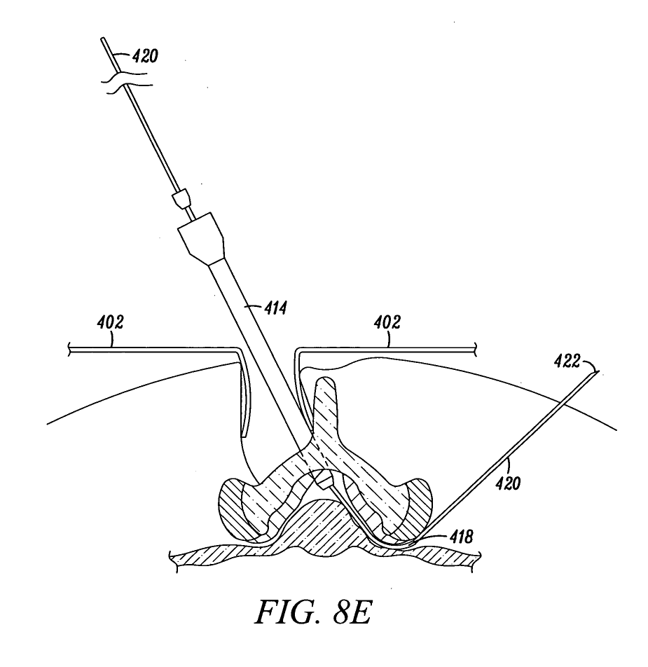

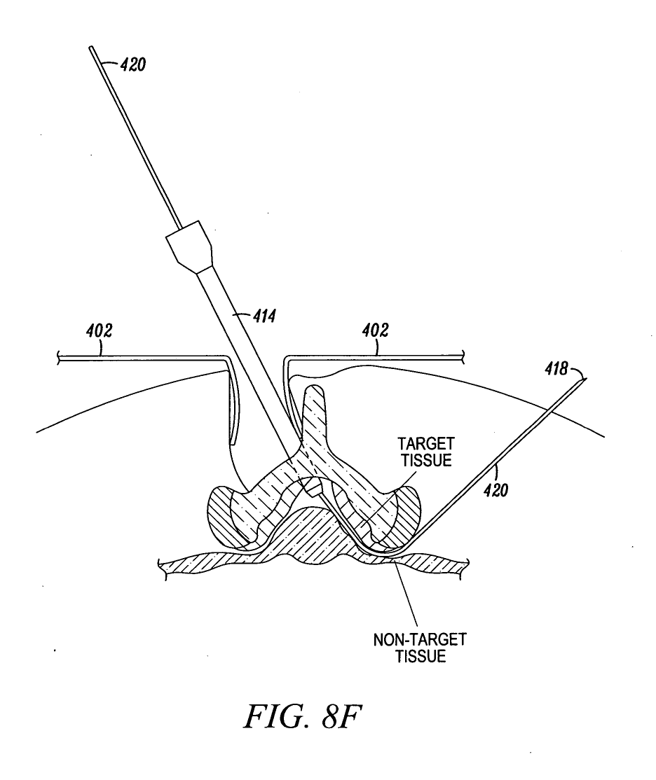

Referring now to FIGS. 8A-8E, in an alternative embodiment, a tissue modification device and optionally one or more introduction/access devices may be positioned in a patient using an open surgical technique. As shown in FIG. 8A, for example, in one embodiment an open surgical incision is made on a patient's back, and two retractors 402 are used to expose a portion of the patient's vertebra. As shown in FIG. 8B, an introducer sheath 414 may then be inserted through the incision, between retractors 402. As in FIG. 8C, a curved guide device 418 may then be inserted through introducer sheath 414. Guide device 418 extends into the epidural space and through the intervertebral foramen as shown in FIG. 8D.

In some embodiments, a curved and cannulated thin, blunt probe may be placed directly through the open incision into the epidural space of the spine, or alternatively may be placed through introducer sheath 414. The probe tip may be advanced to or through a neural foramen. Such a probe may be similar in shape, for example, to a Woodson elevator, Penfield 3, hockey stick probe, ball tipped probe, or the like. In alternative embodiments, probes that may be manually bent to change their shapes, or probes with articulating tips, or probes with shape lock portions, and/or probes having grooves instead of cannulas may be used.

As shown in FIGS. 8D-8E, a substantially straight, flexible guidewire 420 with a sharp tip 422 may then be inserted through curved guide device 418 and advanced so that its distal portion with sharp tip 422 extends outside the patient's back at a location separate from the open incision (FIG. 8E). Guide device 418 may then be removed, as in FIG. 8F, and in subsequent steps a tissue modification device may be inserted over guide wire 420 and through introducer sheath 414 and used to modify tissue as described in more detail above. In an alternative embodiment, a curved, flexible cannula may be inserted through the curved guide device, until it extends lateral to the neural foramen, after which a substantially straight, flexible guidewire with a sharp tip may then be inserted through curved cannula and advanced so that its distal portion with sharp tip extends outside the patient's back.

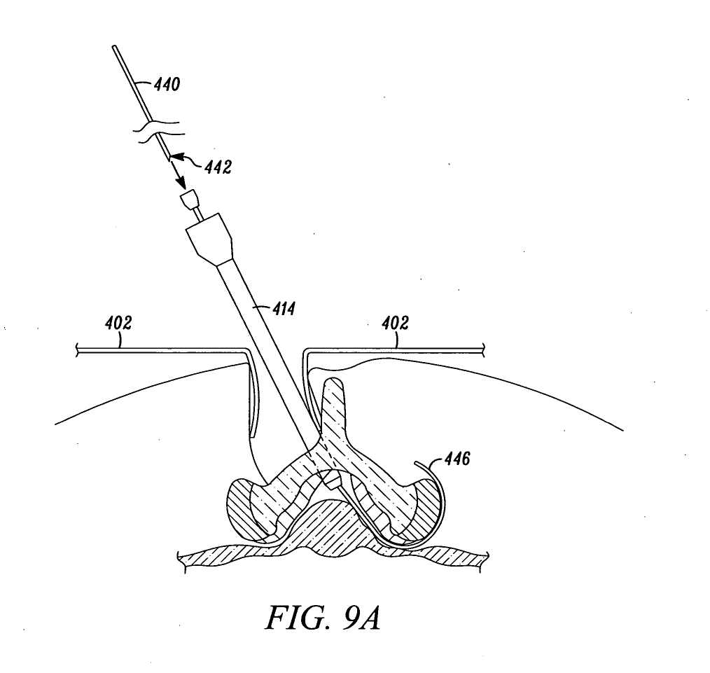

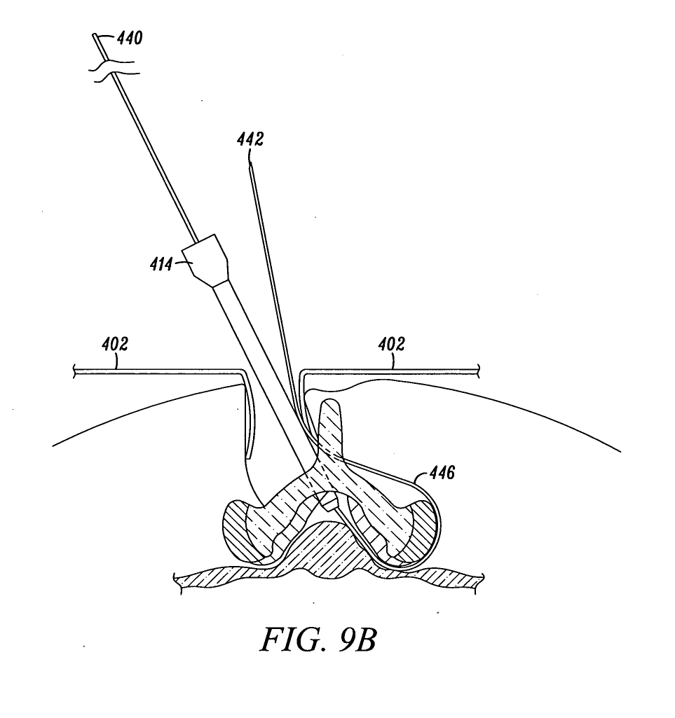

Referring now to FIGS. 9A and 9B, another alternative open surgical access method is shown. In FIG. 9A, a curved guide device 446 is shown in place through the epidural space and intervertebral foramen, and a guidewire 440 with a beveled distal tip 442 is about to be advanced through guide device 446. As shown in FIG. 9B, in this embodiment, guidewire 440 is directed by guide device 446 back through the open incision through which the various access devices are introduced. In such an embodiment, then, only one incision is created and the proximal and distal portions of one or more devices extend out of the patient's back through the same incision.

In various alternative embodiments, open surgical access may be through exposure down to a vertebral lamina, through ligamentum flavum without lamina removal, through ligamentum flavum with partial or complete lamina removal, through ligamentum flavum with or without lamina removal with partial or complete medial facet joint removal, through open exposure and out through skin laterally, through open exposure and back out through the open exposure, or through a lateral open exposure that accesses the neural foramen from the lateral side. One or more visualization devices may be used with open surgical access procedures as well as with percutaneous or other less invasive procedures. In another alternative embodiment (not shown), a tissue modification device may be placed in the patient directly, without any introduction devices.

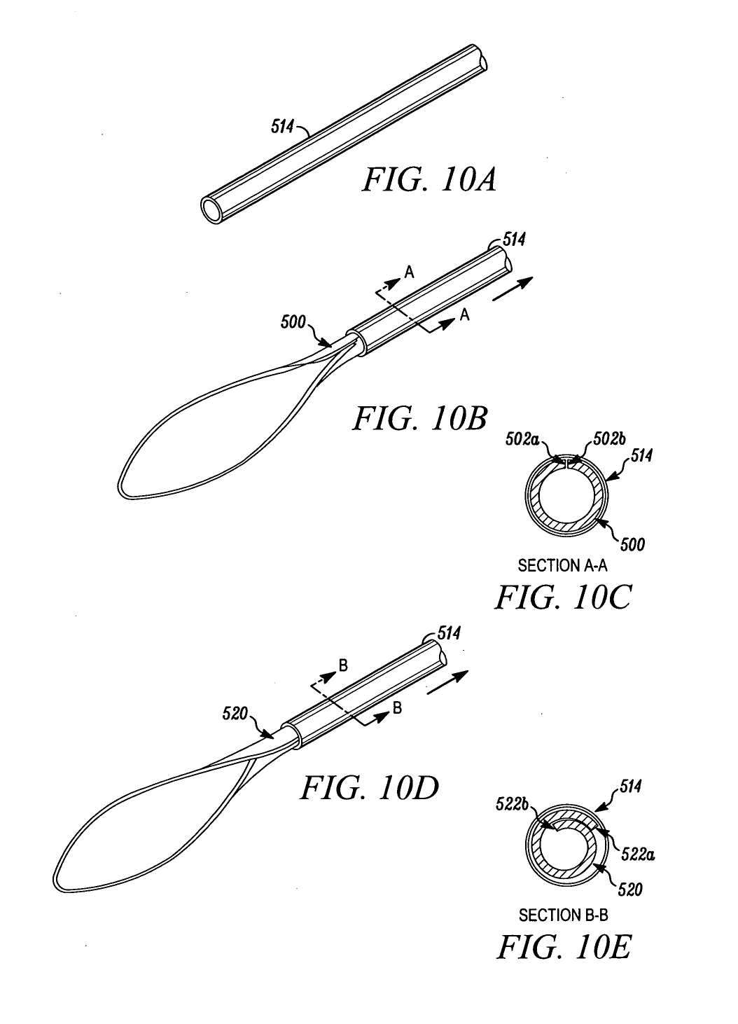

Referring now to FIGS. 10A-10E, in the embodiments described above, the tissue modification devices 102, 202 include at least one non-tissue-modifying (or “protective”) portion, side or surface. The non-tissue-modifying portion is located on tissue modification device 102, 202 so as to be positioned adjacent non-target tissue when tissue modifying members 110, 210 are facing the target tissue. The non-tissue-modification surface of the device is configured so as to not modify or damage tissue, and thus the non-target tissue is protected from unwanted modification or damage during a tissue modification procedure. Alternatively, in some embodiments, a protective surface or portion of tissue modification device 102, 202 may actually modify non-target tissue in a protective manner, such as by delivering a protective drug, coating, fluid, energy or the like to the non-target tissue.

Optionally, in some embodiments, tissue modification devices or systems may further include one or more tissue barriers (or “shields”) for further protecting non-target tissues. Such barriers may be slidably coupled with, fixedly coupled with, or separate from the tissue modification devices with which they are used. In various embodiments, a barrier may be delivered between target and non-target tissues before delivering the tissue modification device, may be delivered along with the tissue modification device, or may be delivered after delivery of the tissue modification device but before the device is activated or otherwise used to modify target tissue. Generally, such a barrier may be interposed between the non-target tissue and one or more tissue modification devices to prevent unwanted damage of the non-target tissue.

FIG. 10A shows a distal portion of an introducer device 514 through which a barrier may be introduced. FIGS. 10B and 10C show one embodiment of a barrier 500 partially deployed and in cross-section, respectively. Typically, barrier 500 will have a first, small-profile configuration for delivery to an area near non-target tissue and a second, expanded configuration for protecting the non target tissue. In various embodiments, barrier 500 may have any of a number of sizes and shapes. For example, barrier 500 is shown in FIG. 10B with a tapered end. In an alternative embodiment, barrier 500 may instead have a squared-off end, a more rounded end, or the like. In fact, many of the embodiments shown in subsequent figures have squared-off ends. Many, if not all, embodiments described herein may have either a tapered end, a squared-off end, a rounded end, or any other suitable shape in alternative embodiments.

In various embodiments, some of which are described more fully below, barrier 500 may be configured as one piece of super-elastic or shape-memory material, as a scaffold with material draped between the scaffolding, as a series of expandable wires or tubes, as a semicircular stent-like device, as one or more expandable balloons or bladders, as a fan or spring-loaded device, or as any of a number of different devices configured to expand upon release from delivery device 514 to protect tissue. As shown in FIGS. 10B and 10C, barrier 500 may comprise a sheet of material disposed with a first end 502a abutting a second end 502b within introducer device 514 and unfurling upon delivery.

In an alternative embodiment, as shown in FIGS. 10D and 10E, opposite ends 522a and 522b of a barrier 520 may overlap in introducer device 514. Generally, barrier 500, 520 may be introduced via introducer device 514 in one embodiment or, alternatively, may be introduced via any of the various means for introducing the tissue modification device, such as those described in conjunction with FIGS. 7A-7S, 8A-8F and 9A-9B. In some embodiments, barrier 500, 520 may be fixedly coupled with or an extension of a tissue modification device. Barrier 500, 520 may also include one or more lumens, rails, passages or the like for passing a guidewire or other guide member, for introducing, removing, steering, repositioning, or exchanging any of a variety of tissue modification, drug delivery, or diagnostic devices, for passing a visualization device, for passing a device designed for neural localization, for providing irrigation fluid and/or suction at the tissue modification site, and/or the like. In some embodiments, barrier 500, 520 is advanced over multiple guidewires and the guidewires remain in place during a tissue modification procedure to enhance the stability and/or maintain positioning of barrier 500, 520.

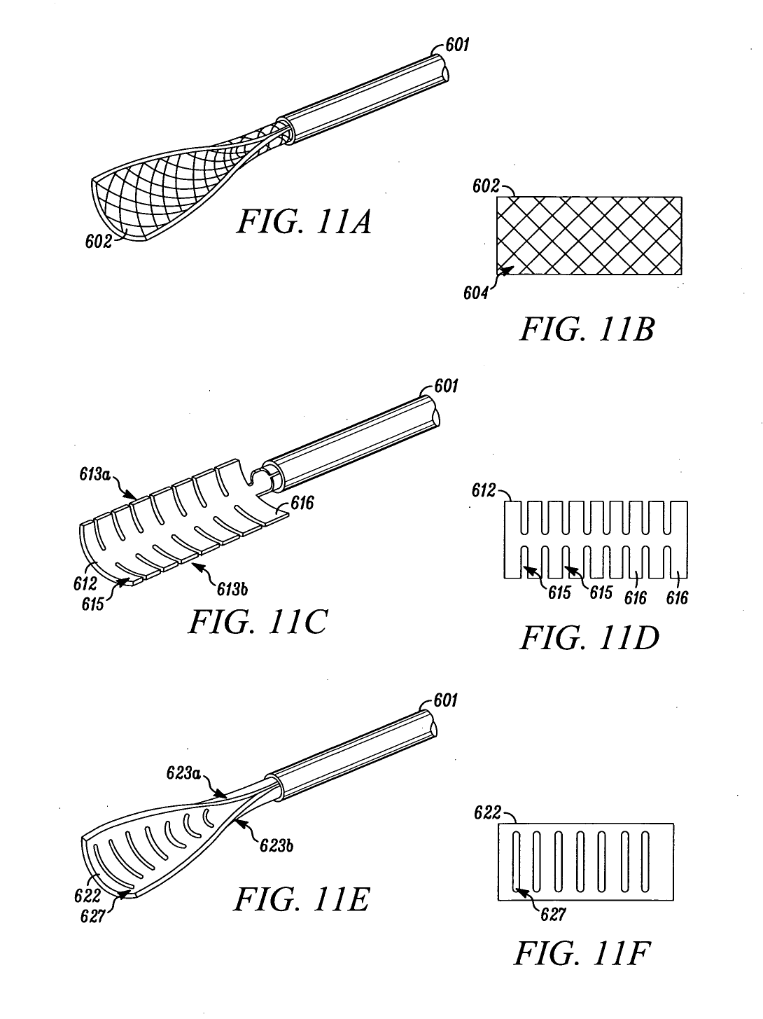

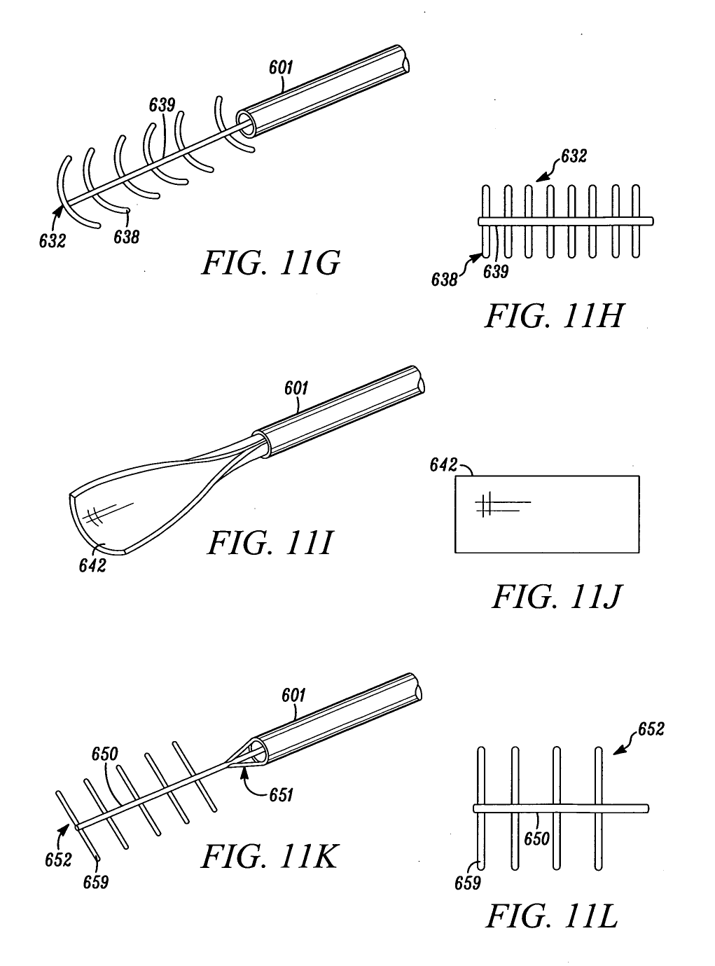

Introducer device 514, which is alternatively referred to as a delivery device 601 in FIG. 11 et seq., may comprise any suitable catheter, introducer, sheath or other device for delivering one or more barrier devices into a patient. In various alternative embodiments, barrier devices may be delivered into a patient either through a delivery device, over one or more guide members, or both. Various guide member embodiments will be described in greater detail below. In various embodiments, introducer device 514 or delivery device 601 may have any suitable dimensions, profile or configuration. For example, in various embodiments, introducer device 514 may have a circular cross-sectional shape, an oval cross-sectional shape, or a shape that varies between circular and oval along the length of device 514. In some embodiments, an outer diameter of introducer device 514 or delivery device 601 may range from about 0.025″ to about 1.0″, with a wall thickness range of about 0.001″ to about 0.125″. Optionally, introducer device 514 or delivery device 601 may taper along its length. Introducer device 514 or delivery device 601 may rigid, partially flexible or flexible along its entire length and may be made from any suitable material, such as but not limited to: a metal, such as stainless steel (303, 304, 316, 316L), nickel-titanium alloy, cobalt-chromium, or nickel-cobalt; a polymer, such as nylon, silicone, polyetheretherketone (PEEK), polyetherketoneketone (PEKK), polytetrafluoroethylene (PTFE), polyurethane (Tecothane), Pebax (co, USA), polycarbonate, Delrin (co, USA), high-density polyethylene (HDPE), low-density polyethylene (LDPE), HMWPE, and UHMWPE; or a combination of metals and polymers. Introducer device 514 or delivery device 601 may be manufactured by methods known in the art, such as CNC machining, extruding, casting, injection molding, welding, RF shaping, electrochemical fabrication (EFAB), LIGA (lithographic, galvanoforming and abforming), electrical discharge machining (EDM) laser machining, silicon micromachining, weaving, braiding or non-woven fabrication techniques (e.g., spunbound, meltblown, and the like). In some embodiments, introducer device 514 or delivery device 601 may be woven from polymer or metal into a tube-like structure for flexibility and conformability. Such embodiments may optionally be fiber-reinforced for added strength to allow for a thinner wall thickness.

Referring now to FIGS. 11A and 11B, an alternative embodiment of a barrier 602 comprising a woven, braided or non-woven material with a lattice structure 604 is shown. FIG. 11A shows barrier 602 being deployed from delivery device 601, and FIG. 11B shows barrier 602 in its completely deployed (expanded, free) configuration. In various embodiments, barrier 602 may have any of a number of suitable dimensions. For example, in some embodiments, barrier 602 may have a width ranging from about 0.100″ to about 3.000″, a length ranging from about 0.100″ to about 72″, and a thickness ranging from about 0.001″ to about 0.250″. In some embodiments, as described in connection with FIGS. 10B and 10D above, barrier 602 may have a narrowed or tapered distal end. Barrier 602 may be manufactured by methods known in the art, such as in a single-layer flat-form or a dual-layer tubular-form that is pressed flat. Material used to fabricate barrier 602, in various embodiments, may be composed of a weave of metallic wire, monofilament or braided. The metallic wire may be made from any suitable material, such as stainless steel (303, 304, 316, 316L), nickel-titanium alloy, cobalt-chromium alloy, Elgiloy® (Elgin Specialty Metals, Elgin, Ill., USA), Conichrome® (Carpenter Technology, Reading, Pa., USA), Phynox® (Imphy SA, Paris, France) or the like. A woven material may be composed of a weave of polymer strands, monofilament or braided material. Polymer strands in a woven, braided or non-woven material construction may be made from nylon, polyester, Dacron®, polyethylene, Kevlar® (DuPont), acetal, Delrin® (DuPont), polycarbonate, nylon, silicone, polyetheretherketone (PEEK), polyetherketoneketone (PEKK), polytetrafluoroethylene (PTFE), polyurethane, UHMWPE, or the like. In some embodiments, barrier 602 may self-expand after being released from a constrained configuration in delivery device 601. In some embodiments, such self-expansion may be achieved by forming barrier 602 from a shape-memory or super-elastic material.

Referring to FIGS. 11C and 11D, in an alternative embodiment, a barrier 612 may include multiple slits 615 extending from opposite edges 613a, 613b toward the longitudinal center of barrier 612 to form multiple tabs 616. Slits 615 may enhance flexibility of barrier 612 by allowing tabs 616 to flex independently. Tabs 616 may then return to their flat-form state individually as delivery device 601 is pulled proximally, as shown in FIG. 2D. Tabs 616 may also conform individually to surrounding tissue, thereby helping protect non-target tissue, in some embodiments. Barrier 612 may be made of any suitable material, such as but not limited to those described above, and slits 615 may be formed by any suitable method, such as die cutting, milling machining, laser cutting, EDM machining, injection molded, etching, waterjet cutting, and blade cutting. In an alternative embodiment, barrier 612 may be made by assembling multiple tabs 616 to a central member by welding, soldering, brazing, or laser welding, for example.

FIGS. 11E and 11F illustrate another alternative embodiment of a barrier 622 having slits 627 disposed more centrally and not extending to the lateral edges 623a, 623b.

In another alternative embodiment, shown in FIGS. 11G and 11H, a barrier 623 comprises a central support member 639 and multiple lateral ribs 638 that form a skeleton-like framework. In various embodiments, central member 639 and ribs 638 may either comprise the same material or different materials, and any suitable materials may be used, such as but not limited to the materials listed above. In some embodiments, ribs 638 may retain a curvilinear shape after deployment that is heat-set in nickel-titanium or mechanically formed, as shown in FIG. 11G.

Referring to FIGS. 11I and 11J, in another alternative embodiment, a barrier 642 may comprise a flat-form sheet made from polymer, porous polymer, woven or non-woven fabric, metal, porous metal, foam, hydrogel, a double-layer polymer “bag” to create an inflatable bladder, or the like.

Referring to FIGS. 11K and 11L, in another alternative embodiment, a barrier 652 may comprise a central support member 650 and ribs 659 that straighten completely or nearly completely upon deployment. Optionally, barrier 652 may also include a flex-point 651 at which barrier 652 may articulate.

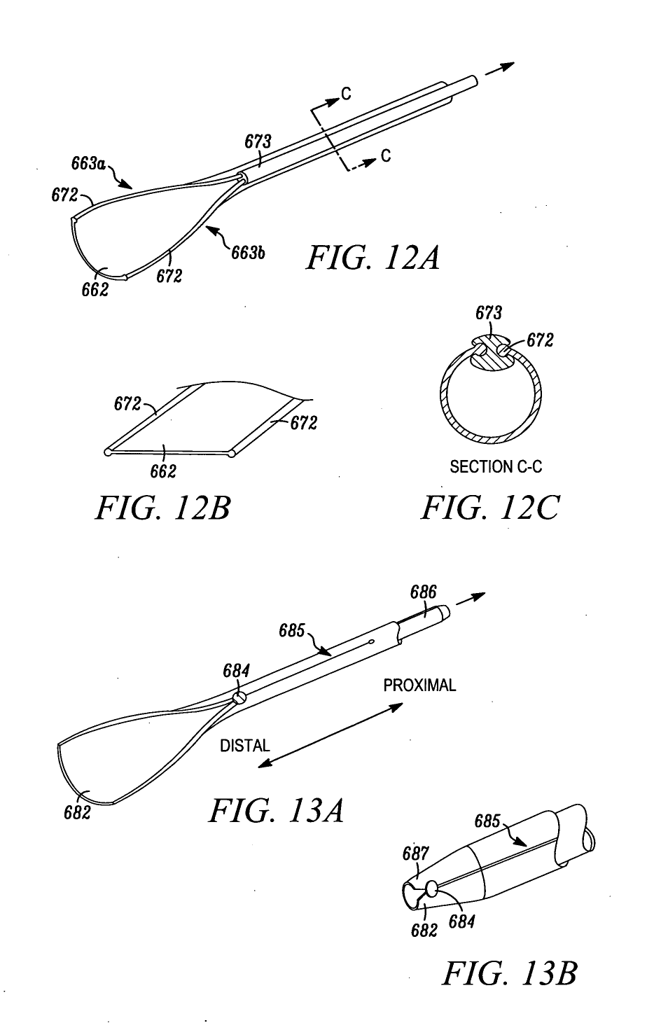

With reference now to FIGS. 12A-12C, in one embodiment a barrier 662 may include ridges 672 disposed along opposite lateral edges 663a, 663b. Ridges 672 may be configured to engage with and slide through recessed channels (or grooves) in a guide number 673, as shown in FIG. 12C. In this embodiment, guide member 672 may be used for advancing barrier 662 into a patient, and it may not be necessary to use a sheath or catheter-type delivery device. Optionally, guide member 673 may include one or more reinforcing members (not shown) to give it strength and stiffness. In some embodiments, such reinforcing members may have a pre-set shape (such as an arc) along their length, which may help guide barrier 662 into a desired location. Examples of reinforcing elements include, but are not limited to, a wire, a hypotube, a monofilament, braided polymer, and the like.

Referring to FIGS. 13A and 13B, in another alternative embodiment, a barrier 682 may suitably include a groove 685 that may be split by a separating member 684 to deploy barrier 682. Separating member 684 may be coupled with an inner cannula 686 (or rod), which may be retracted (arrow) to cause separating member 684 to split barrier 682 along groove 685. In some embodiments, as shown in FIG. 13B, barrier 682 may include a tapered distal end 687 to facilitate advancement of barrier 682 into a patient and/or through a delivery device. Separating member 684 may have any suitable shape and configuration and may comprise, in various embodiments, a post, a blade, a wedge, or the like.

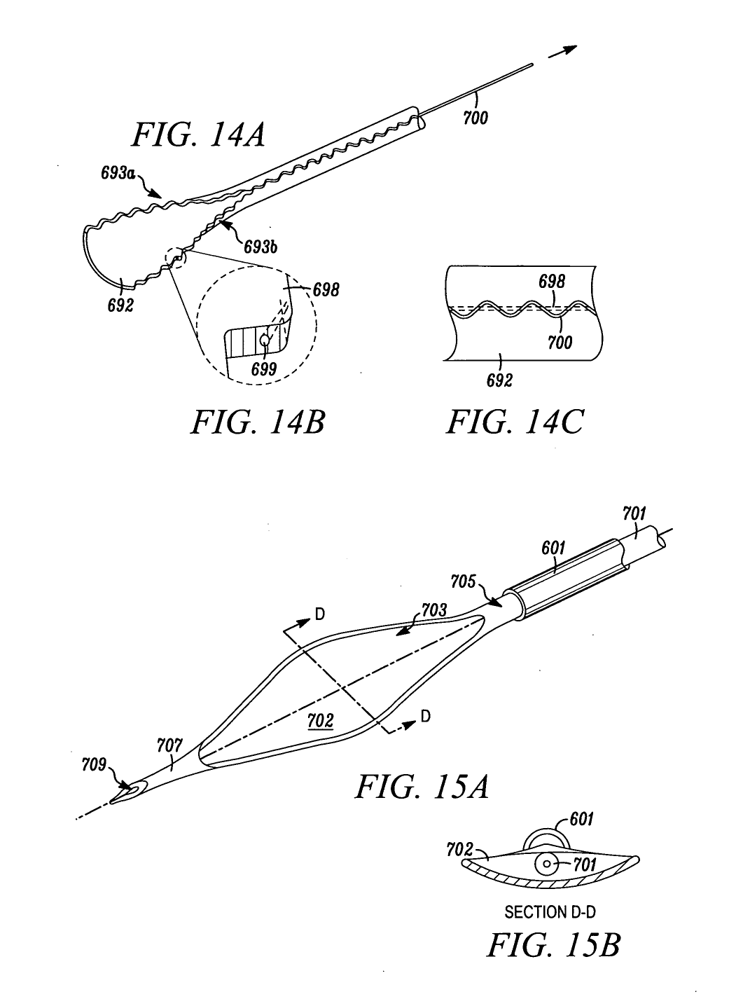

FIGS. 14A-14C show another embodiment of a barrier 692, including a zipper-like seam comprising multiple interdigitating teeth 698, which join together the opposite edges 693a, 693b of barrier 692. As shown in FIG. 14B, teeth 698 include a port-699 approximately aligned with the longitudinal axis of barrier 692, which accommodates a pull rod 700. When pull rod 700 is retracted proximally, as in FIG. 14A, teeth 698 are freed from one another and the zipper-like seam unzips to deploy barrier 692. Teeth 698 may have any of a number of shapes, such as but not limited to triangular (as shown), rectangular, curvilinear, or any other interdigitating (nesting, interlocking) geometry. In various embodiments, pull rod 700 may be made of metal or polymer, monofilament or braided.

Referring now to FIGS. 15A and 15B, in one alternative embodiment, a barrier device 702 may suitably include an expandable or shape changing portion 703, a tapered distal portion 707, an elongate proximal portion 705, and a guidewire lumen 709 to allow passage of barrier device 702 over a guidewire 701. In some embodiments, barrier device 702 may be passed over guidewire 701 and through a delivery device 601, as shown in FIG. 15B. In various embodiments, shape changing portion 703, distal portion 707 and proximal portion 705 may have any desired lengths, widths and thicknesses. For example, in one embodiment barrier device 702 may have an overall length such that proximal portion 705 may extend outside a patient through a first entry point, and distal portion 707 may extend outside a patient through a second entry point, while shape changing portion 703 resides between target and non-target tissue, such as tissue in a spine or other location in the body. In such an embodiment, tensioning and/or anchoring forces may be applied to both proximal portion 705 and distal portion 707 so that shape changing portion 703 may urge part of a tissue modification device against target tissue. In alternative embodiments, only proximal portion 705 or only distal portion 707 may have sufficient length to extend outside a patient while shape changing portion 703 is in position between target and non-target tissues. In some embodiments, barrier device 702 may be fabricated from a single piece of material.

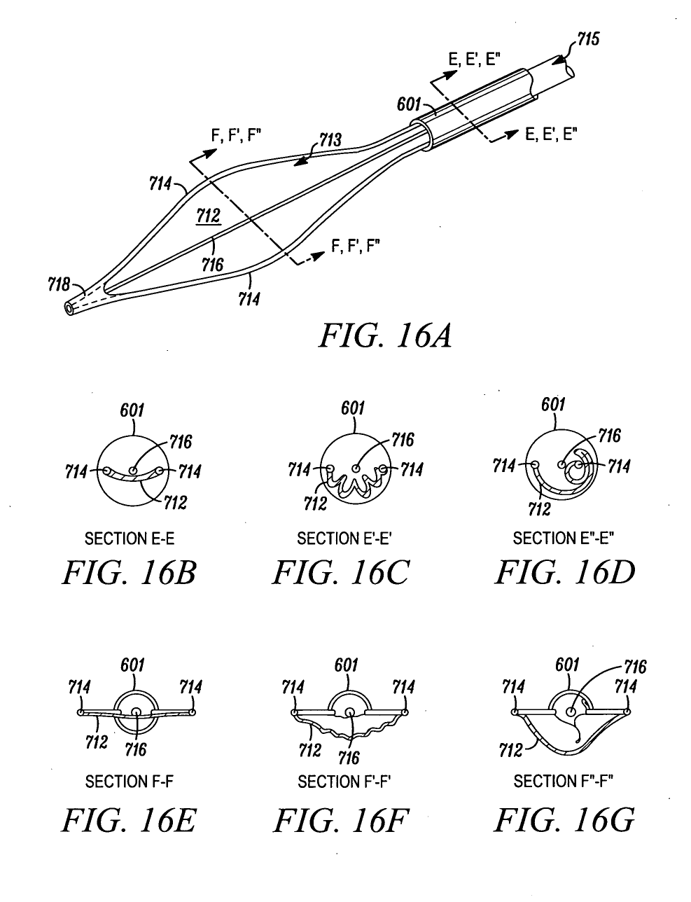

Turning now to FIGS. 16A-16G, in another embodiment, a barrier device 712 may include a shape changing portion 713, a distal portion 718, and a proximal portion 715. Shape changing portion 713 may include frame comprising a central support member 716 and lateral support members 714, with material disposed over, around or between them. Central support member 716 may help support shape changing portion 713 and may also act to expand the frame. In some embodiments, central support member 716 may have a tubular configuration to accommodate a guidewire. Alternatively, lateral support members 714 may be pre-shaped or biased to expand automatically when deployed from a delivery device 601. Lateral members 714, for example, may comprise hypotubes, flat material, solid rods, or braided wires, in various embodiments. Distal portion 718 may be formed and attached to central support member 716 and lateral support members 714 by methods know in the art, for example, resistance welding, over-molding, brazing, laser-welding, or the like. In various embodiments, barrier device 712 may be housed in delivery device 601 in any of a number of configurations, such as but not limited to those shown in FIGS. 16B-16D. In one embodiment, barrier device 712 may be stored in delivery device 601 in a flat configuration, as shown in the cross-sectional view of FIG. 16B, and may be stretched when deployed from delivery device 601, as depicted in FIG. 16E. Alternatively, barrier device 712 may be a stored in delivery device 601 in a folded (ruffled) configuration, as shown in the cross sectional view of FIG. 16C, and may be unfolded when deployed from delivery device 601, as depicted in FIG. 16F. Alternatively, barrier device 712 may be a stored in delivery device 601 in a rolled (overlapped) configuration, as shown in the cross sectional view of FIG. 16D, and may be unrolled when deployed from delivery device 601, as depicted in FIG. 16G. As shown, in various embodiments, shape changing portion 713 of barrier device 712 may assume any of a number of suitable deployed configurations.

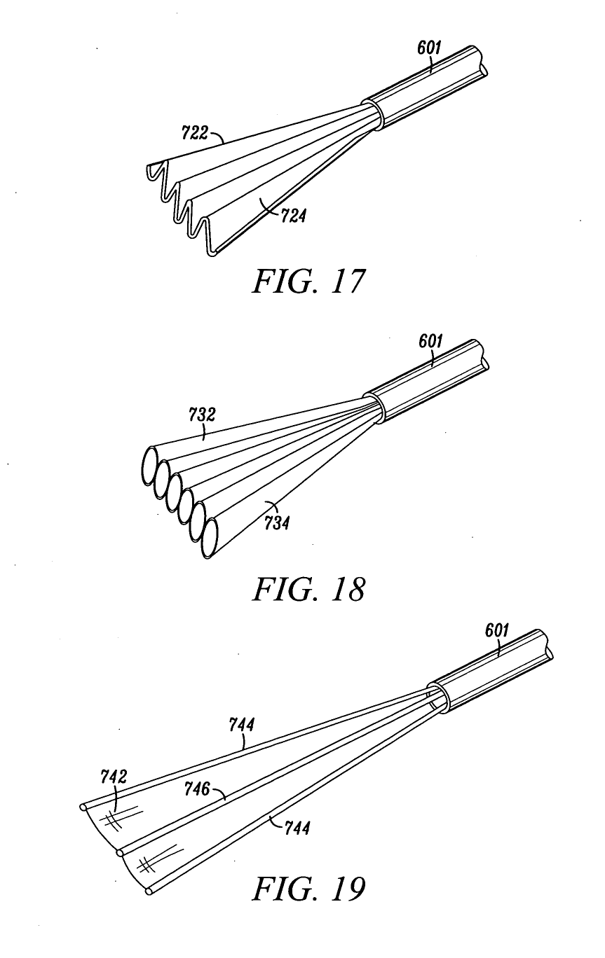

Referring to FIG. 17, in another alternative embodiment, a barrier device 722 may have a fan-like or corrugated configuration, including multiple bends 724, folds, hinges, creases or the like. In various embodiments, barrier device 722 may open automatically upon extending out of a delivery device 601 or, alternatively, may be opened with the use of one or more actuators. In various embodiments, bends 724 may be formed by bending and yielding the material to take a permanent set or by bending elastically to the point where the material does not take a set and returns to the original state when unconstrained. Bends 724 may also be formed as “live” hinges. A “live hinge” is a groove (edge, line, trough) of reduced thickness used to create a more flexible region in a body of material, which may be produced using various techniques known in the art. The stiffness and thickness of barrier device 722 may be adjusted in various embodiments to provide desired self-expanding properties. In various alternative embodiments, barrier device 722 may have a narrowed, tapered or rounded distal end instead of the squared-off end shown in FIG. 17.

In an alternative embodiment, as shown in FIG. 18, a barrier device 732 may include multiple, attached, compliant tubes 734, which may be compressed to fit within a delivery device 601, and which expand when released from delivery device 601. In one embodiment, tubes 734 may be made of polymer and may be bonded, fastened, RF welded, attached together with adhesive, or the like. In an alternative embodiment, barrier device 732 may be extruded as a single piece of material. In alternative embodiments, the distal ends of tubes 734 may be sealed and pressure applied to the proximal ends may be used to expand barrier device 732 when it exits deliver device 601. In alternative embodiments, either of barrier device 722 or 732 may have a distal portion at which the device tapers to a more low-profile configuration, as in the barrier devices of FIGS. 15 and 16.

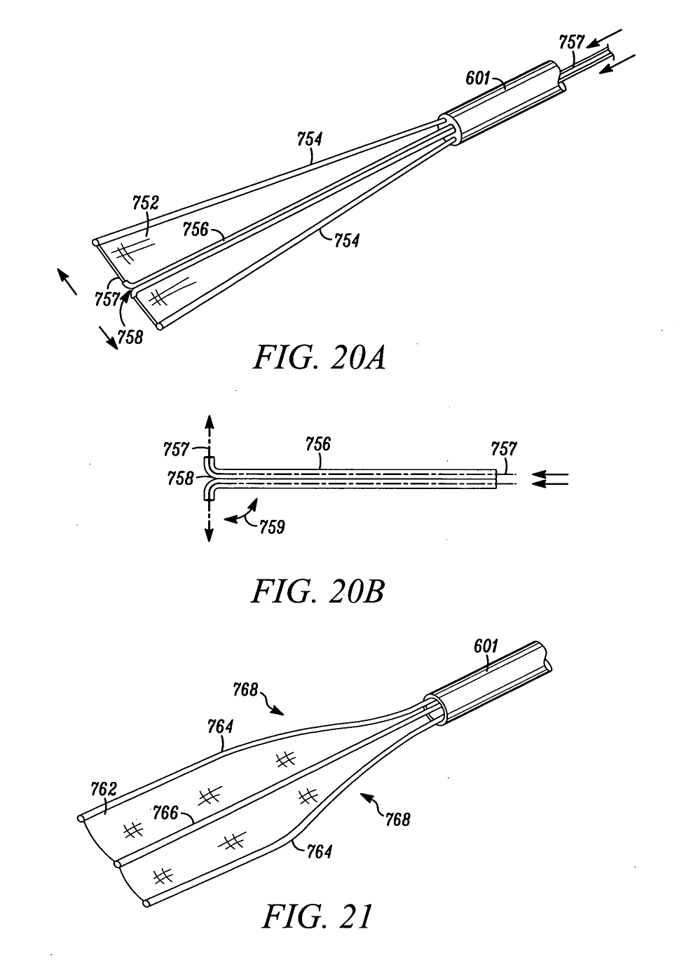

Referring to FIG. 19, in another embodiment, a barrier device 742 may include a straight central support member 746 and straight lateral support members 744, with material covering or stretched between the members 746, 744. In some embodiments, as shown in FIGS. 20A and 20B, a barrier device 752 may include a central support member 756, lateral support members 754, and a push rod 757 ending in a diverter 758. Diverter 758 may act to redirect the applied force from the proximal end of push rod 757 to apply an outward force on lateral support members 754, thereby expanding barrier device 742. Central member 756 may include a lumen or dual-lumen to allow passage of push rod 757 therethrough. Diverter 758 may have any suitable angle in various embodiments, such as between about 45 degrees and about 135 degrees, or more preferably between about 75 degrees and about 115 degrees.

In yet another embodiment, and with reference now to FIG. 21, a barrier device 762 may include a central support member 766 and lateral support members 764 having bends 768 to increase barrier device's 762 surface area. In various embodiments, lateral support members 764 may have any number of bends, arcs or geometries to enhance functionality of barrier device 762. Bends 768 may be included in the design of barrier device 762 as provided from the manufacturer or may be modified by a surgeon or other user to customize barrier device 762 during a procedure.

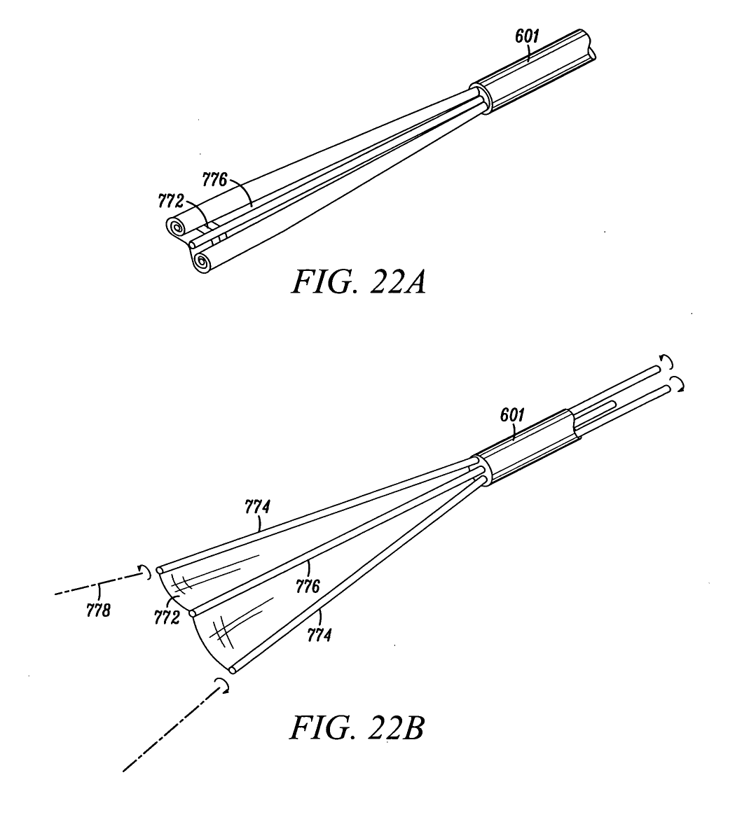

Referring to FIGS. 22A and 22B, in another embodiment, two halves of a barrier device 772 may be rolled from lateral support members 774 toward a central support member 776, to assume a low-profile configuration for delivery through a delivery device 601. When barrier device 772 is exposed out the distal end of delivery device 601, each lateral support member 774 may be turned about an axis 778 to unroll barrier device 772.

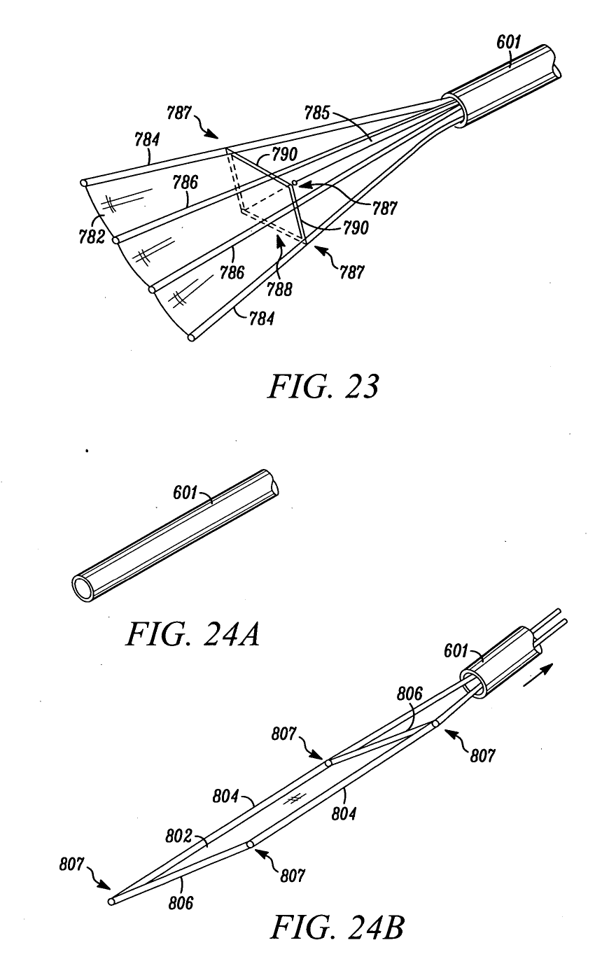

In another embodiment, as shown in FIG. 23, a barrier device 782 may include a scaffold and material draped over or between elements of the scaffold. For example, barrier device 782 may include multiple central support members 786 and lateral support members 784, and an articulated mechanism 788 including multiple linking members 790 and hinges 787. Articulated mechanism 788 may be expanded and collapsed, for example, via an actuator 785, which may comprise a pull wire, push rod or the like in various embodiments. In one embodiment, articulated mechanism 788 may apply an outward force when articulator 785 is advanced in a distal direction. Alternatively, articulated mechanism 788 may apply an outward force when articulator 785 is retracted in a proximal direction.

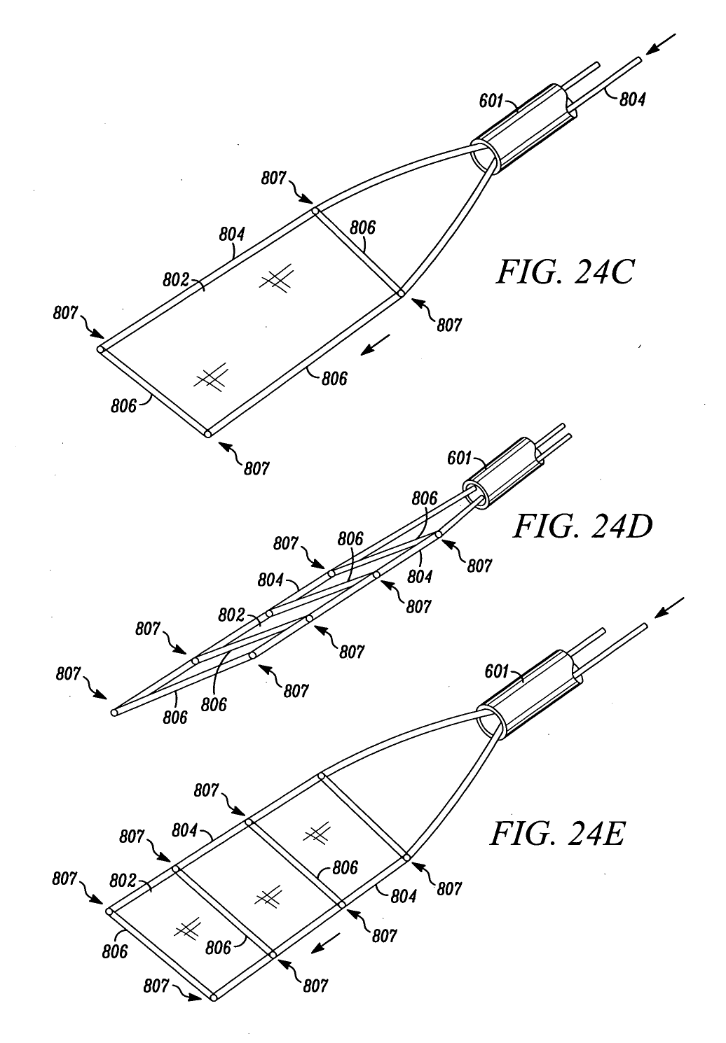

With reference now to FIGS. 24A-24E, two additional alternative embodiments of a barrier device 802, which may be advanced through a delivery device 601 are shown. In some embodiments, delivery device 601 completely houses barrier device 802 before deployment, as in FIG. 24A. In the embodiment shown in FIGS. 24B and 24C, barrier device 802 includes a “4-bar linkage” including two longitudinal support members 804 and two transverse support members 806, all coupled together via multiple hinges 807, flexure points, or pivot points. As shown in FIG. 24B, one longitudinal support member 804 may be retracted proximally (arrow) to collapse barrier device 802. As shown in FIG. 24C, one longitudinal support member 804 may also be advanced distally to expand barrier device 802. In some embodiments, support members 804, 806 may be rigid, while in alternative embodiments some or all may be flexible. In an alternative embodiment (not shown), transverse support member 806 most proximal to delivery device 601 may be eliminated to create a “3-bar linkage” mechanism. In yet another embodiment, as shown in FIGS. 24D and 24E, additional transverse support members 806 may be added to barrier device 802 to provide additional support.

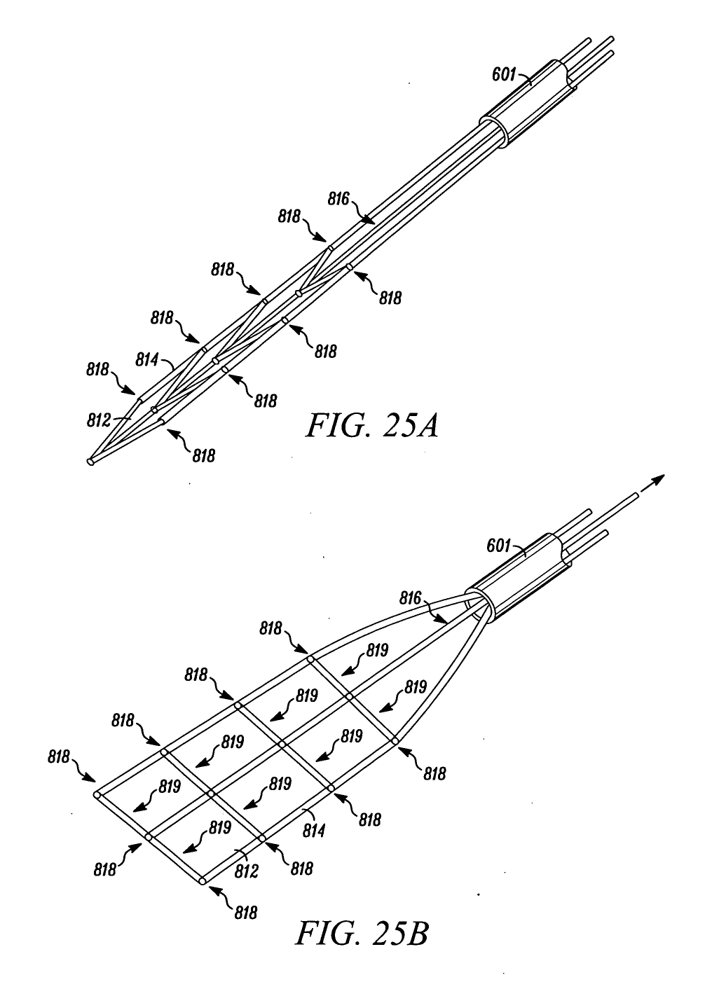

In another alternative embodiment, and referring now to FIGS. 25A and 25B, to provide additional support, a barrier device 812 may include even more transverse support members 819, joined to a central support members 816 and lateral support members 814 by a hinges 818 (or pivots, flexure points or the like). In some embodiments, pulling central support member 816 may cause barrier member 812 to expand (FIG. 25B), and pushing central support member 816 distally may cause barrier device 812 to collapse (FIG. 25A). In an alternative embodiment, pulling central support member 816 may cause barrier device 812 to collapse, and pushing central support member 816 distally may cause barrier device 812 to expand. In alternative embodiments, to create a curvature in the plane of barrier device 812, the transverse support members 819 may have arc-like shapes. In various embodiments, a flexible material or membrane may cover, be stretched between, or otherwise be coupled with support members 814, 816, 819.

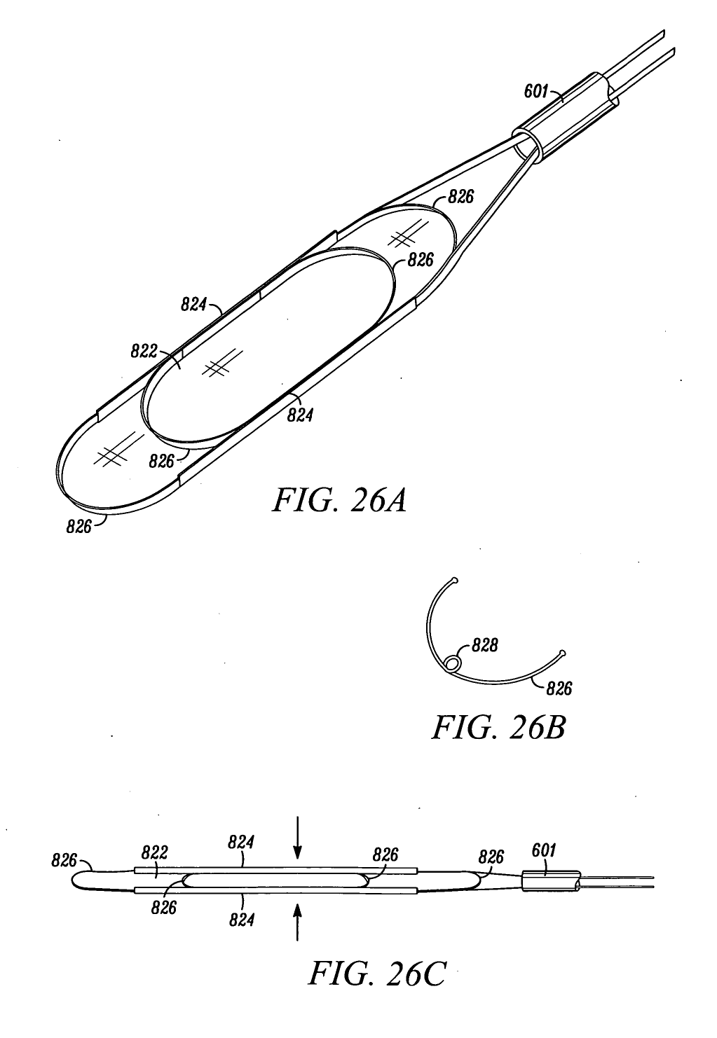

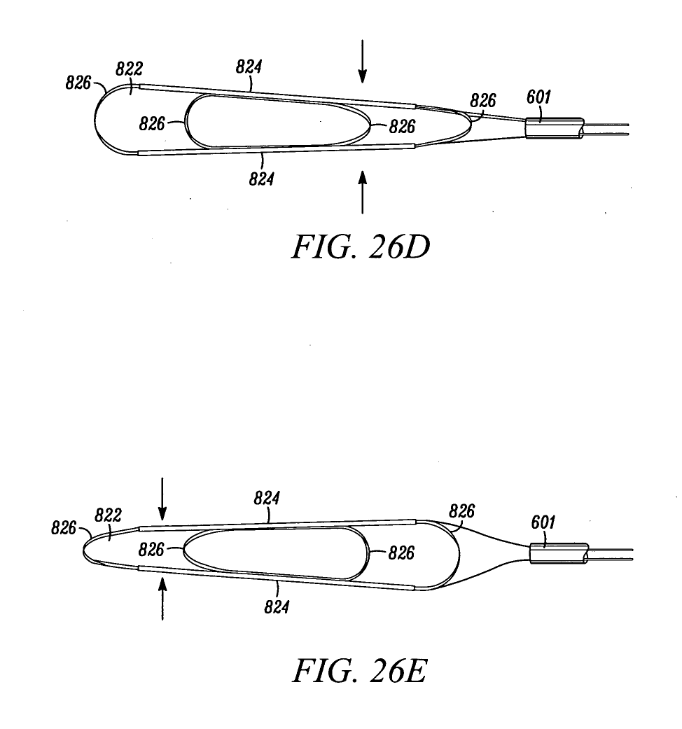

Referring to FIGS. 26A-26E, in another embodiment, a barrier device 822 may include lateral support members 824 coupled with flex-linkages 826. One version of a flex-linkage 826 may be formed from wire, as shown in FIG. 26B, with a central loop 828 to provide strain relief. Flex-linkages 826 may deform resiliently when lateral support members 824 impart an inward force, either during manipulation in a surgical field or as a delivery device 601 is advanced distally, as shown in the various configurations of barrier device 822 depicted in FIGS. 26C-26E.

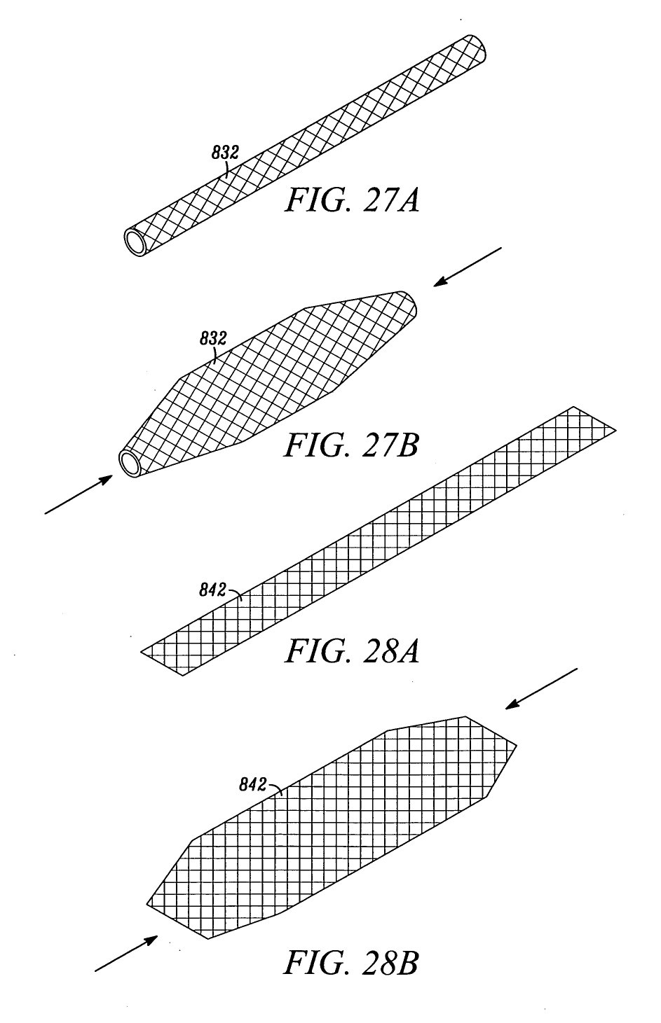

FIGS. 27A and 27B illustrate another alternative embodiment of a barrier device 832, in which device 832 comprises a tubular, woven mesh. Barrier device 832 may assume an elongate, low-profile configuration, as in FIG. 27A, to facilitate its delivery to a treatment area, and may also be compressed from one or both ends to assume a widened/expanded configuration for protecting tissue, as in FIG. 27B. In another embodiment, as in FIGS. 28A and 28B, a barrier device may comprise a flat woven mesh.

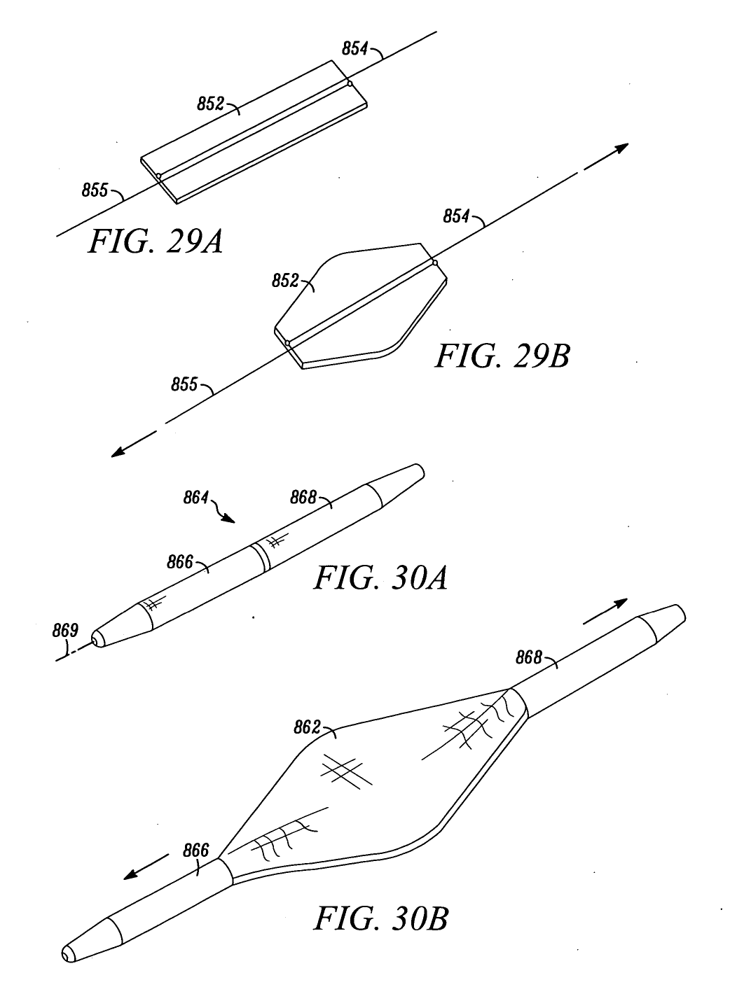

Another alternative embodiment of a barrier device 852 is depicted in FIGS. 29A and 29B. Here, a first pull wire 854 and a second pull wire 855, extending from opposite ends of a shape changing portion of barrier device 852, may be pulled to cause the shape changing portion to expand or widen (FIG. 29B). In some embodiments, when pull wires 854, 855 are released, the shape changing portion may resume its original, narrower configuration (FIG. 29A).

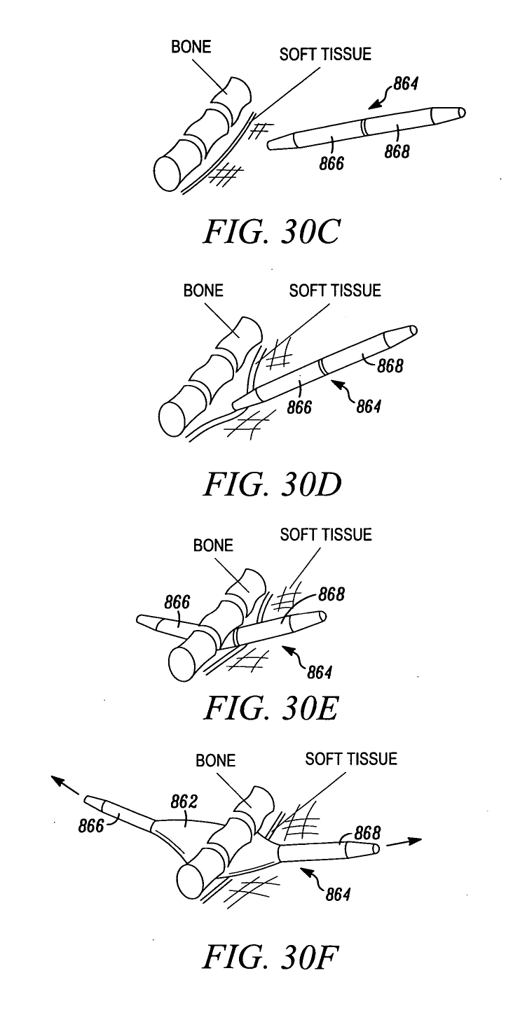

Referring now to FIGS. 30A-30F, in another embodiment, a barrier device 862 may be housed in a housing 864 comprising two halves 866, 868, and a lumen for allowing passage of a guidewire 869. When halves 866, 868 are pulled apart, as in FIG. 30B, barrier device 862 is free to expand. FIGS. 30C-30F illustrate a method for deploying barrier device 862 between target and non-target tissue, such as bone and soft tissue. In FIGS. 30C and 30D, housing 864 is positioned between the bone and soft tissue. In FIGS. 30E and 30F, halves 866, 868 are pulled apart to expose barrier device 862 and thus allow it to expand. In various embodiments, housing 864 may have an atraumatic (or blunt) end or ends and may be advanced to a position between tissues using any of a number of suitable methods. For example, housing 864 may be advanced by itself between the tissues, may be advanced over one or more guidewires or other guide members, may be advanced through a delivery sheath or other delivery device, or some combination thereof.

In another embodiment, as shown in FIGS. 31A-31C, a barrier device 872 may include a woven wire structure including lateral straight wires 874 coupled with crossing wires 877, 878 via multiple loops 876. In one embodiment, lateral wires 874 slide freely through loops 876, to allow barrier device 872 to collapse and expand. Wires 876, 877, 878 may be coupled with end caps 880, 881 at either end of barrier member 872. Some embodiments may also include pull tabs 879, 882 at either end of barrier member 872. As shown in FIG. 31C, when pull tabs 879, 882 are pulled, barrier device 872 may shorten and expand to a wider configuration. As shown in FIGS. 31A and 31C, when pull tabs 879, 882 are pulled, an angle between cross wires 877, 878 decreases. In an alternative embodiment, pulling pull tabs 879, 882 may cause barrier device 872 to collapse. In some embodiments, wires 874, 877, 878 themselves may perform the protective function of barrier member 872, while in alternative embodiments a material or membrane may be coupled with wires 874, 877, 878.

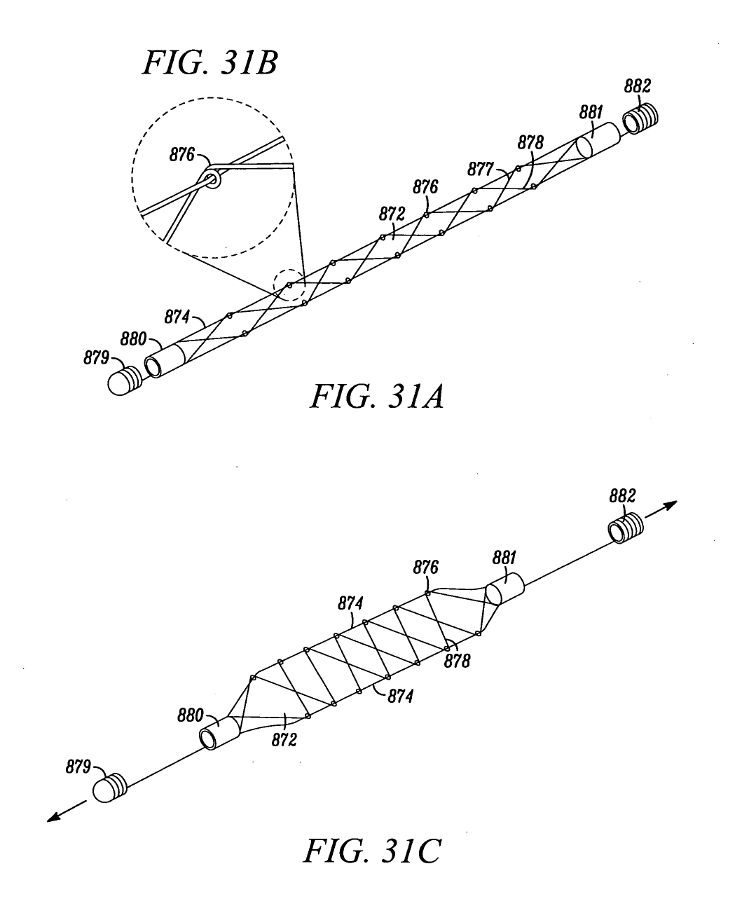

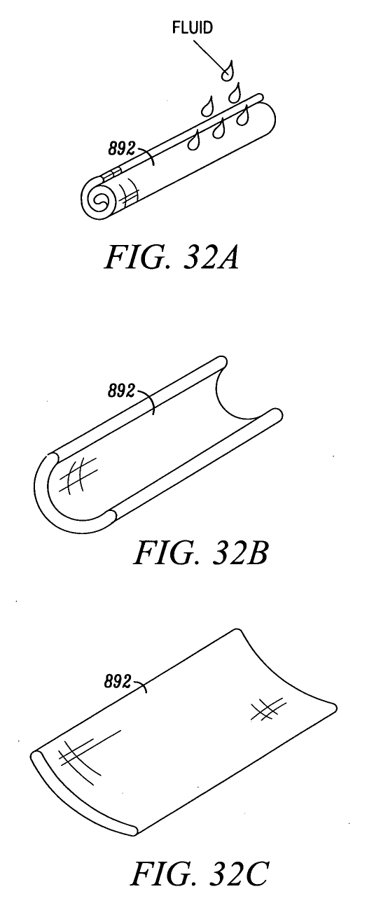

Referring now to FIGS. 32A-32C, in another alternative embodiment, a barrier device 892 may include a piece of hydrogel material, which expands and/or unrolls from a collapsed/rolled configuration (FIG. 32A) to an expanded/unrolled configuration (FIG. 32C) when exposed to one or more fluids, such as saline, water, blood or the like. In one embodiment, hydrogel may be injected directly into an area between target and non-target tissues to form barrier device 892, and device 892 may be left in the patient's body to dissolve after a tissue modification procedure is complete. In other alternative embodiments, barrier device 892 may comprise one or more alternative self-expanding materials or materials that expand upon exposure to fluid.

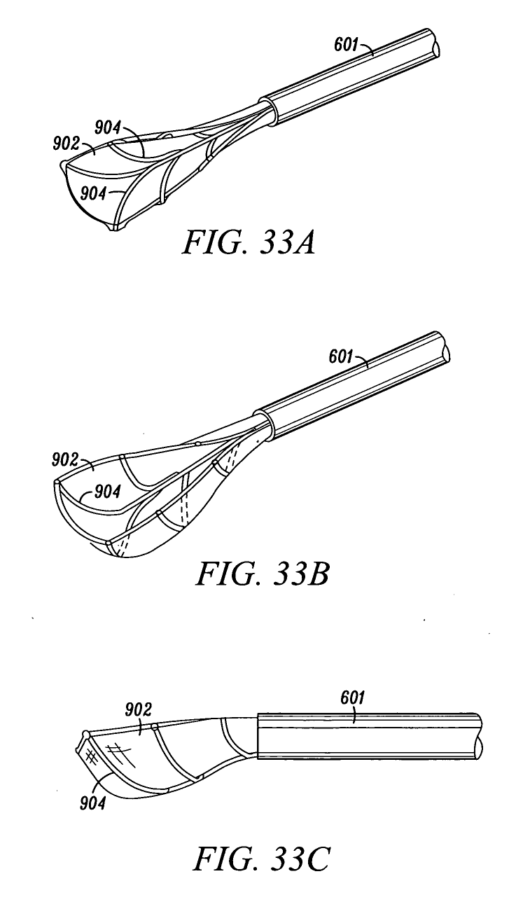

In yet another embodiment, as shown in FIGS. 33A-33C, a barrier device 902 may have a cup-like or scoop-like shape formed by multiple support members 904 coupled with a material or membrane. This embodiment of barrier device 902 may function and be fabricated in a similar manner to a number of the embodiments described above. The cup-like shape may enhance the ability of barrier device 902 to protect multiple surfaces of non-target tissue.

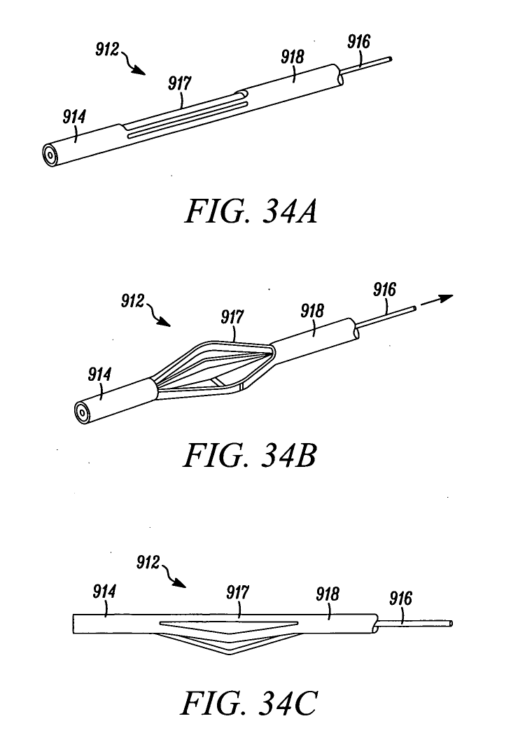

FIGS. 34A-34C show another alternative embodiment, in which a barrier device 912 has a stent-like configuration including multiple expandable/collapsible slats 917 disposed between tubular portions 914, 918 at either end. In one embodiment, a pull wire 916 may be pulled to cause slats 917 to flex, thus expanding barrier device 912 (FIGS. 34B and 34C). In various embodiments, pulling pull wire 916 may cause all or only a subset of slats 917 to expand. As shown in the side view of FIG. 34C, for example, if a subset of slats 917 is expanded, barrier device 912 may form a cup-like shape that may be used to forcibly displace tissue or create additional space in a surgical field. In alternative embodiments, slats 917 may self-expand or may expand via some other mechanism, such as a push rod or other actuator. In other alternative embodiments, any number, size or shape of slats may be incorporated into barrier member 912. In some embodiments, barrier device 912 may be delivered to a desired location in a patient without use of a sheath or catheter delivery device but instead simply over a guidewire. In other embodiments, such as when slats 917 are self-expanding, barrier member 912 may be delivered through a sheath or catheter.

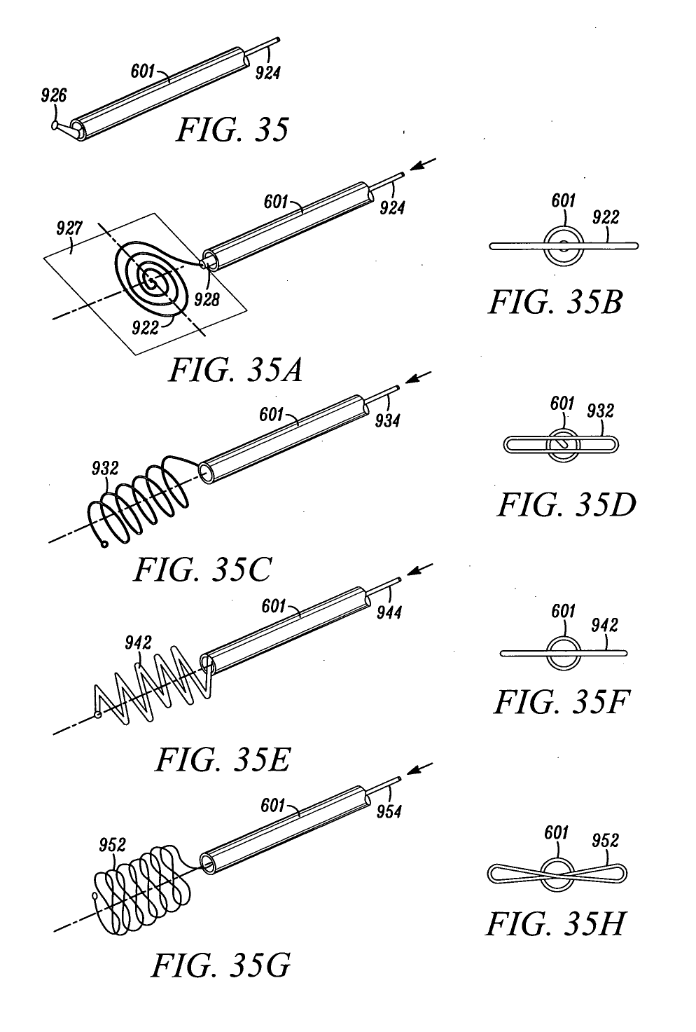

With reference now to FIGS. 35-35H, a number of various embodiments of shaped-wire barrier devices 922, 932, 942, 952 are shown. As seen in FIG. 35, in one embodiment a delivery device 601 may completely or almost completely house a barrier member. A cap member 926 may be exposed out of the distal end of delivery device 601, and a barrier member may be pushed distally out of delivery device to expose a shape changing portion that self-expands (FIGS. 35A, 35C, 35E and 35G). FIGS. 35A and 35B are perspective and end-on views of one embodiment of a barrier device 922 having a flat, spiral shape changing portion that resides predominantly in one plane 927 and a proximal portion 924, which may be used to advance barrier device 922 through delivery device 601. In alternative embodiments, barrier device 922 may be preformed to have a certain shape when released from constraint or, alternatively, device 922 may be deflected by a deflection member 928 to assume a shape.

FIGS. 35C-35H are perspective and end-on views of various embodiments of shaped-wire barrier devices 932, 942, 952, each including a shape changing distal portion and a proximal portion 934, 944, 954 for advancing the barrier device. As seen in the figures, a shaped-wire barrier device may have a helical shape (932, FIGS. 35C and 35D), a zig-zag shape (942, FIGS. 35E and 35F), or an overlapping loop shape (952, FIGS. 35G and 35H). In alternative embodiments, shaped-wire barrier devices may have any of a number of other suitable shapes, sizes or configurations.

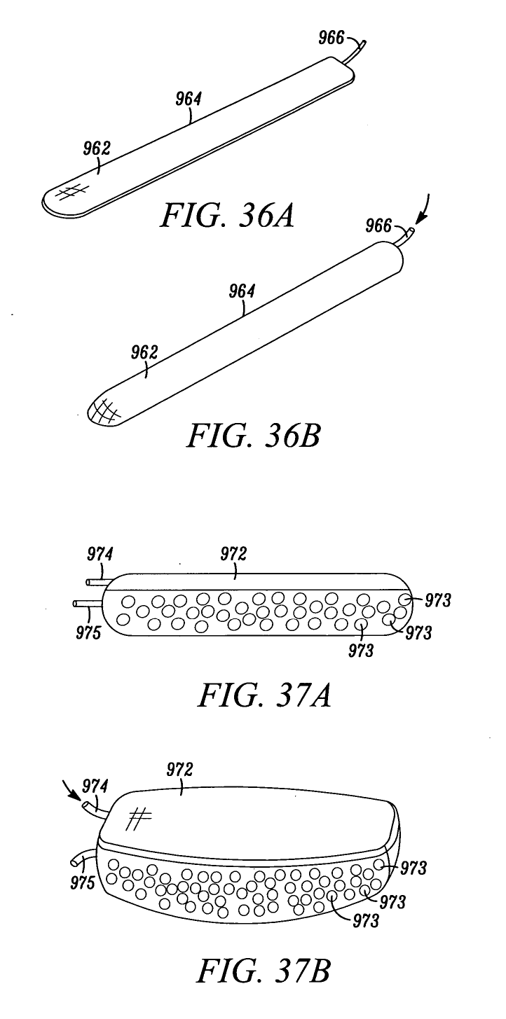

In still another embodiment, and with reference now to FIGS. 36A and 36B, a barrier device 962 may comprise one or more expandable bladders 962, balloons or the like, which may be expanded by introduction of a gas, liquid or solid expansion medium via an filling tube 966. In various embodiments, for example, bladder 962 may be filled and caused to expand (FIG. 36B) using an expansion medium such as air, carbon dioxide (CO2), nitrogen (N2), water, saline, silicone oil, hydrogel, powder, particulate, beads, or any of a number of other media. As bladder 962 is filled via filling tube 966, bladder 962 may both expand and become firmer.

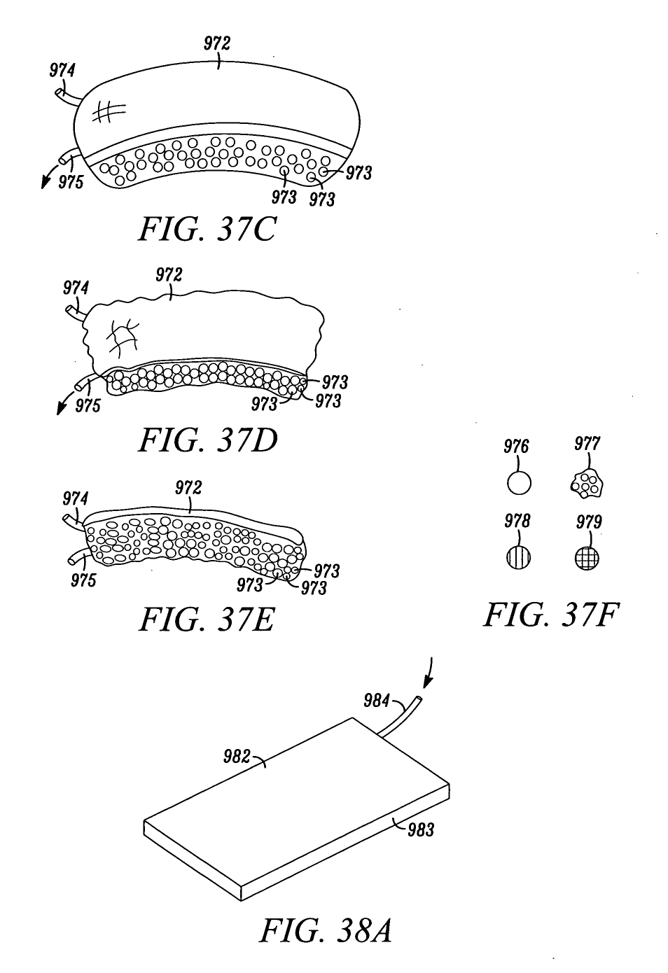

Referring to FIGS. 37A-37E, in one embodiment, an expandable bladder barrier device 972 may include multiple solid particles 973 disposed within device 972, an inflation tube 974 and a suction tube 975. Air, other gases, saline, water or the like may be introduced into bladder barrier device 972 through inflation tube 974, thus causing separation of particles 973 and making device 972 flexible and adjustable, as in FIGS. 37A-37C. Device 972 may be adjusted into a desired shape when flexible, and then air, fluid or the like may be removed from device 972, using suction tube 975, thus bringing particles 973 closer together and making device 972 more solid/firm, as in FIGS. 37D and 37E. Any suitable particles 973 may be used, in various embodiments, and particles 973 may have any of a number of suitable shapes, such as those shown in FIG. 37F. For example, smooth particles 976, rough particles 977, particles with parallel grooves 978 or particles with crossing grooves 979 may be used in various embodiments. Of course, other particles may be used in other alternative embodiments, and particles may be made of any suitable material, such as metal, polymer, ceramic, bioabsorbable material, or the like. The size of the particles 973 may vary from that of a fine powder to a larger particulate with a diameter of about 0.250″, for example.

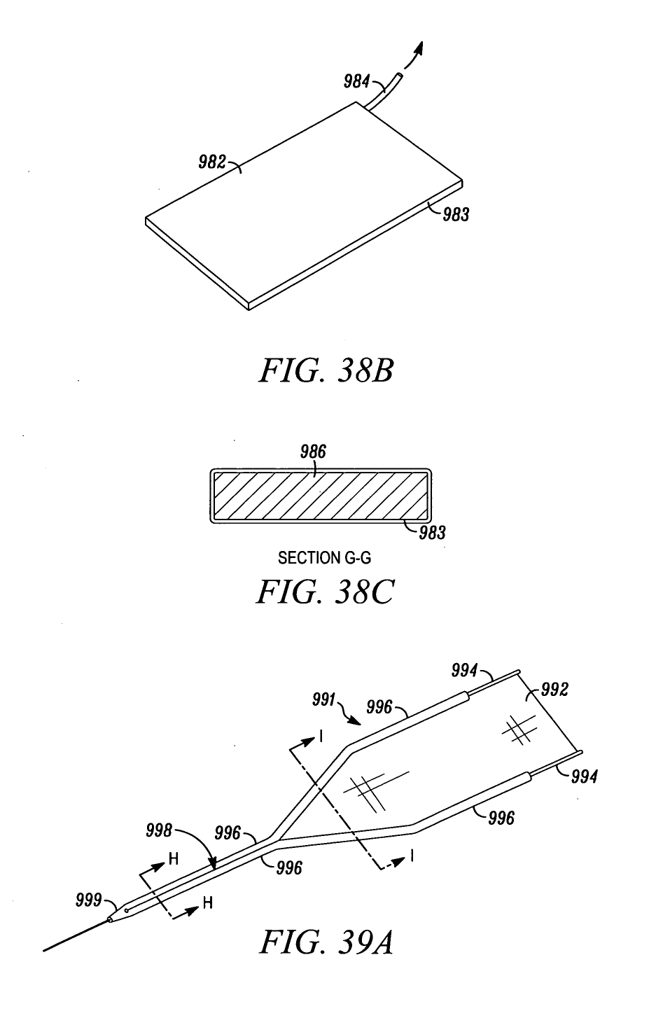

With reference to FIGS. 38A-38C, in another embodiment, a barrier device 982 may include a bladder 983 and a fill tube 984 controlled by a valve (not shown). Bladder 983 may contain a compliant foam material 986 (FIG. 38C, cross-sectional view), which may allow barrier device 982 to unfurl and/or expand by opening the valve of fill tube 984 to allow air, fluid or the like to enter bladder 983, as shown in FIG. 38A. Alternatively or additionally, air, fluid or the like may be forced into bladder 983 through fill tube 984 by positive pressure. Air, fluid or the like may be removed from bladder 983 by applying vacuum via fill tube 984, thus causing barrier member 982 to collapse/deflate to facilitate storage, rolling, delivery through a delivery device and/or the like. In some embodiments, foam material 986 may be bonded to the inside of bladder 983, such that the shape of expanded barrier device 982 may be constrained in a desired configuration.

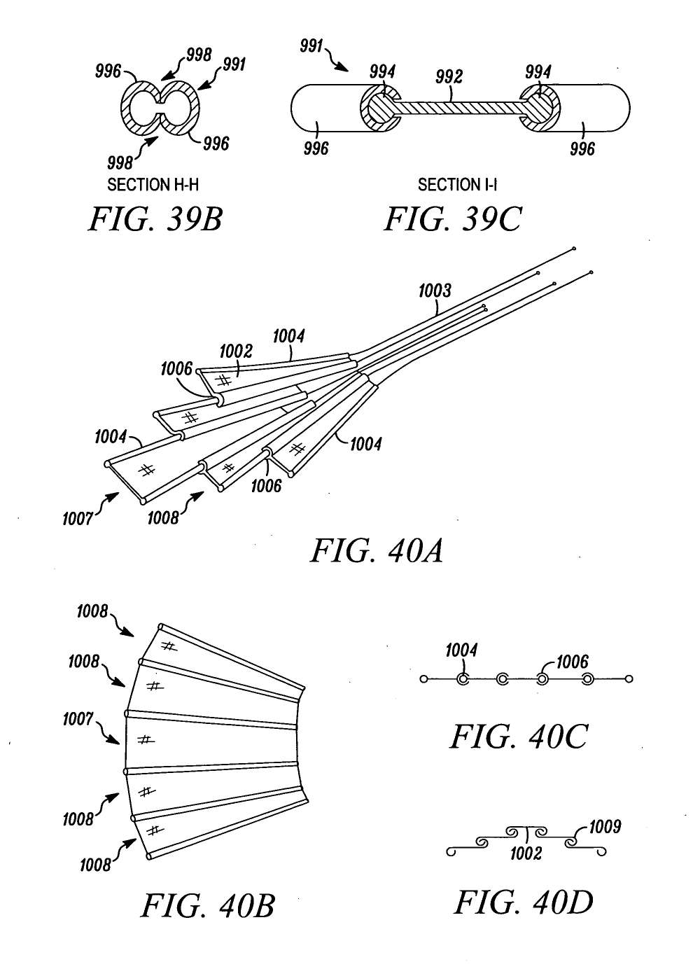

Referring to FIGS. 39A-39C, in another embodiment, a barrier device 992 having two lateral support members 994 may be delivered using a dual-lumen delivery device 991. The two lumens of delivery device 991 may be formed by two tubes 996, which may be joined along part of their lengths, thus forming a groove 998 (FIG. 31A and cross-section 31B), and may be divided along a different portion of their lengths (FIG. 31A and cross-section 31C). In various embodiments, tubes 996 may have any suitable sizes, shapes or configurations and may be made of any suitable material. Optionally, in one embodiment, tubes 996 may coalesce into a common lumen at a tapered distal tip 999. Barrier device 992 may be shaped like a wedge and have lateral support members 994 sized and shaped to slide through lumens formed by tubes 996.

In two additional alternative embodiments, as shown in FIGS. 40A-40D, a barrier device 1002 may include a central wedge 1007 and multiple lateral wedges 1008, with each wedge 1007, 1008 comprising lateral support members 1004 configured to slide through channels 1006 of adjacent wedges 1007, 1008. FIGS. 40A and 40C are perspective and end-on views, respectively, of an embodiment of barrier device 1002 in which wedges 1007, 1008 slide in the same plane. FIGS. 40B and 40D are perspective and end-on views, respectively, of an embodiment of barrier device 1002 in which wedges 1007, 1008 slide in different planes. A control rod 1003 extends proximally from each lateral support member 1004, to allow for positional adjustment wedges 1007, 1008. In various embodiments, wedges 1007, 1008 may have any suitable number, size or shape. In the embodiment shown in FIGS. 40B and 40D, wedges 1007, 1008 are slidably coupled together via corresponding rolled edges 1009.

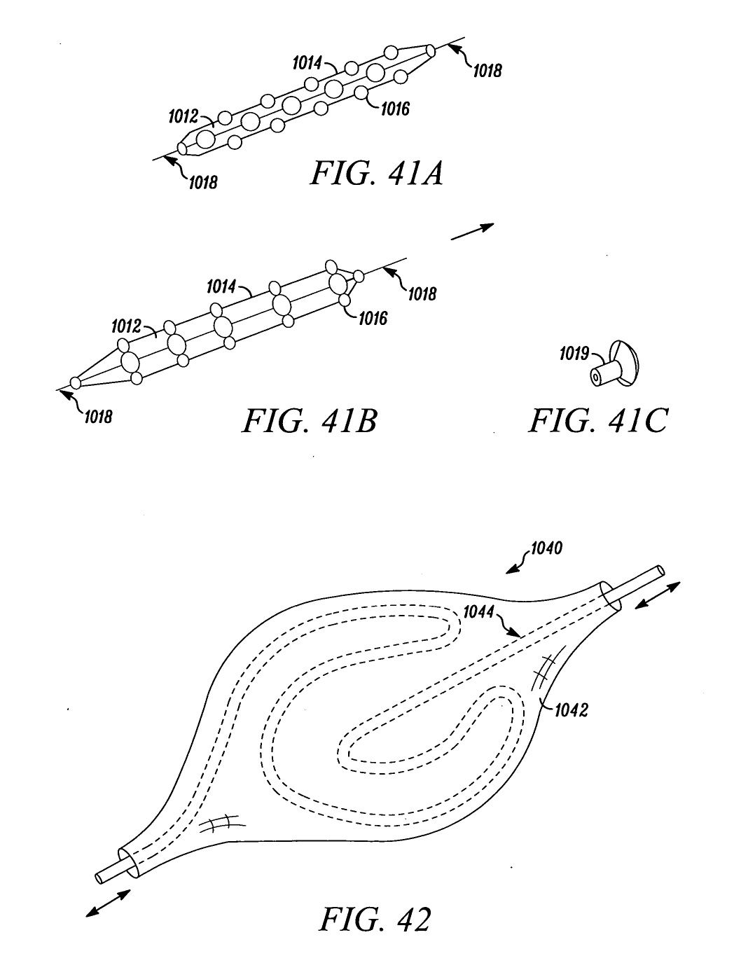

In another alternative embodiment, as shown in FIGS. 41A-41C, a barrier device 1012 may suitably include a central support member 1018, lateral support members 1014, and multiple protrusions 1016 coupled with support members 1018, 1014. When protrusions 1016, which are shown as spherical but may have any suitable shape and size in various alternative embodiments, are out of alignment with one another (i.e., not contacting adjacent protrusions 1016), as in FIG. 41A, barrier device 1012 assumes a narrower configuration. When central support member 1018 is retracted proximally, as in FIG. 41B (arrow), protrusions 1016 align (or “nest”) and thus contact one another to expand/widen barrier device 1012. In alternative embodiments, protrusions 1016 may be brought into alignment/contact by advancing central member 1018, advancing or retracting lateral members 1014 or by any other method. In an alternative embodiment, protrusions 1019 may comprise a boss and a triangular cross-section, as shown in FIG. 41C, which may facilitate the un-nesting process.

Referring to FIG. 42, in yet another embodiment, a barrier device 1040 may include a sleeve of material 1042 covering a shaped wire 1044. Wire 1044 may be adjustable from a straight configuration for delivery to a shaped configuration (as shown) for protecting tissue. In some embodiments, wire 1044 may be pushed and/or pulled from opposite ends (double-headed arrows) to change its shape, while in other embodiments wire 1044 may automatically change its shape upon release from constraint inside a delivery catheter or other delivery device.

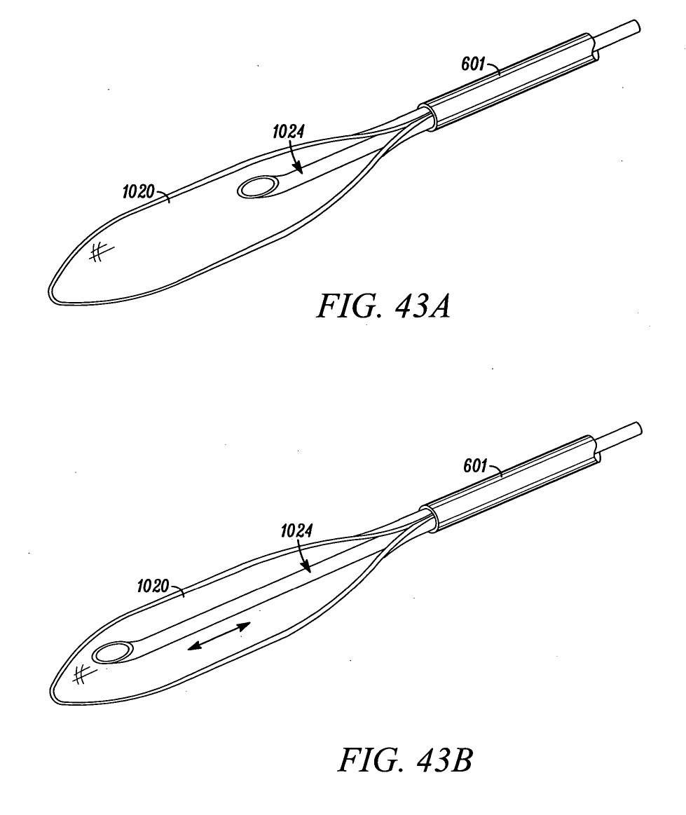

FIGS. 43A and 43B illustrate how, in one embodiment, a barrier device 1020 extending through a delivery device 601 may help protect tissue during a tissue modification procedure involving use of a tissue modification device 1024. In various embodiments, tissue modification device 1024 may include, but is not limited to, a rongeur, a curette, a scalpel, one or more cutting blades, a scissors, a forceps, a probe, a rasp, a file, an abrasive element, one or more small planes, an electrosurgical device, a bipolar electrode, a unipolar electrode, a thermal electrode a rotary powered mechanical shaver, a reciprocating powered mechanical shaver, a powered mechanical burr, a laser, an ultrasound crystal, a cryogenic probe, a pressurized water jet, or any combination of such devices. Tissue modification device 1024 may be advanced and retracted (double-headed arrows) freely on one side of barrier device 1020 and may be used to modify tissue, while barrier device 1020 protects non-target tissue from sustaining unwanted damage. In some embodiments, barrier device 1020 may also be used to help guide tissue modification device 1024 to and/or from a position for performing a tissue modification procedure. Such guidance may be achieved by a shape, surface characteristic and/or one or more guide features of barrier device 1020, according to various embodiments.

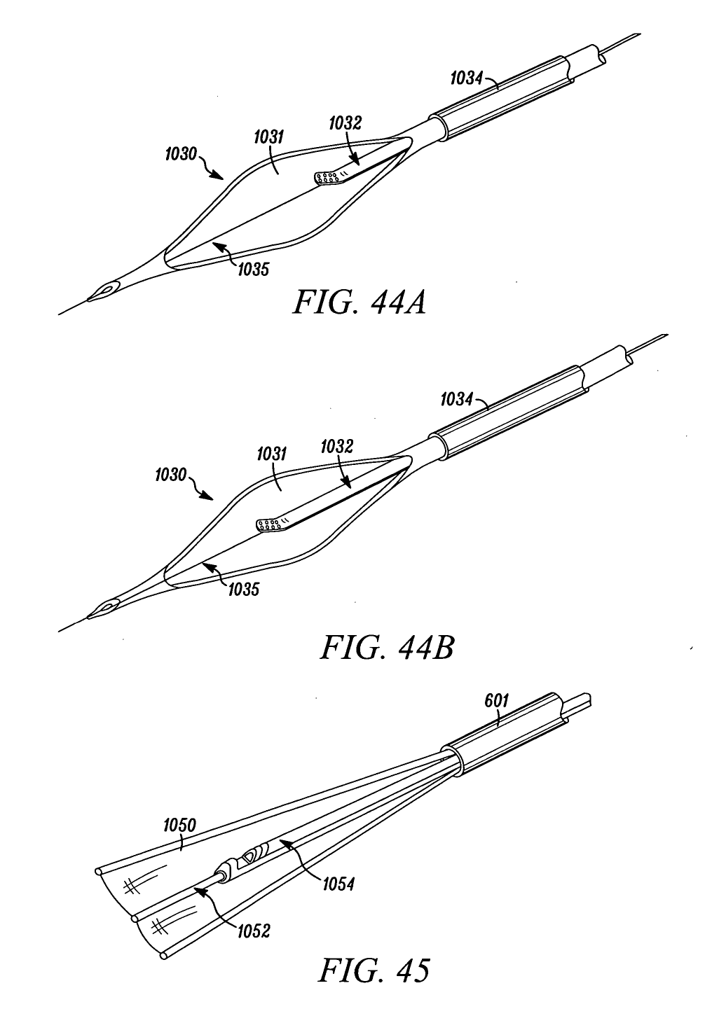

Turning to FIGS. 44A and 44B, in another embodiment, a barrier device 1030 may include an open, shape-changing portion 1030, closed, elongate extensions 1034 extending from either end of shape-changing portion 1030, and at least one guide feature 1035 extending through its length. Guide feature 1035 may include, in various embodiments, one or more guidewires (as shown), rails, impressions, lumens, tracks or the like, any of which may facilitate guidance of a tissue modification device 1032 along and/or through barrier device 1030. In various embodiments, guide feature 1035 may comprise a separate device, not attached to barrier member 1030, as in the guidewire of FIGS. 44A and 44B. Alternatively, one or more guide features 1035 may be attached to, or integral with, barrier member 1030.

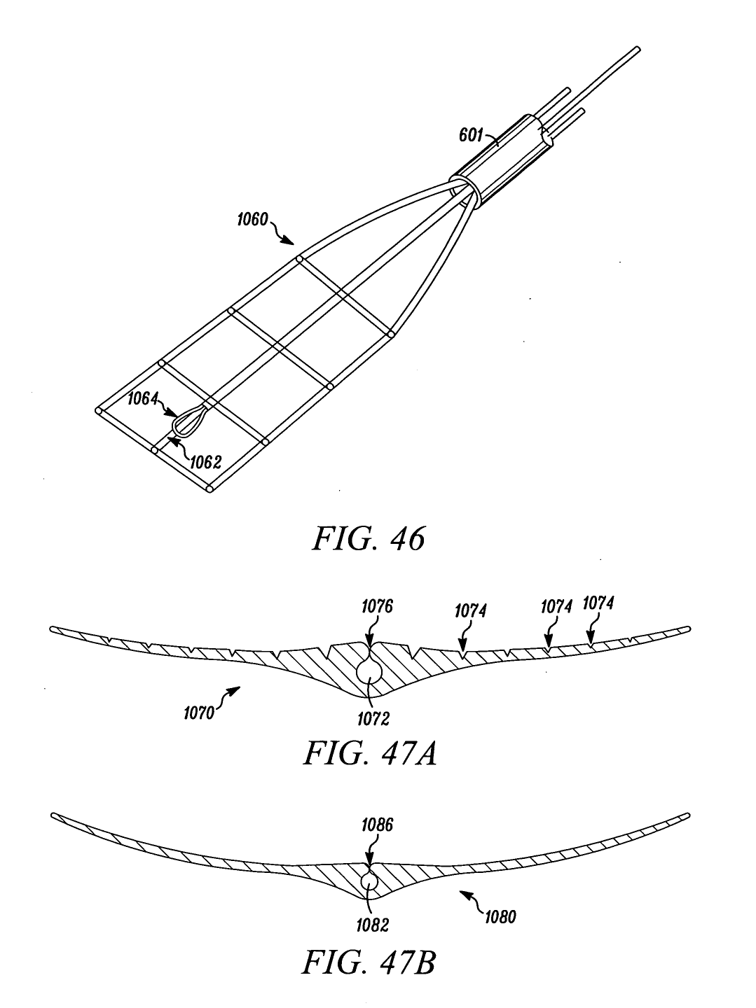

FIG. 45 shows an embodiment of a barrier device 1050 including a central rail 1052 guide member along which a tissue modification device 1054 may be guided. FIG. 46 shows an alternative embodiment of a barrier device 1060 including a central rail 1062 guide member along which a tissue modification device 1064 may be guided. In some embodiments, barrier devices 1050, 1060 and tissue modification devices 1054, 1064 may be advanced through a delivery device 601, while other embodiments may not employ such a delivery device 601.

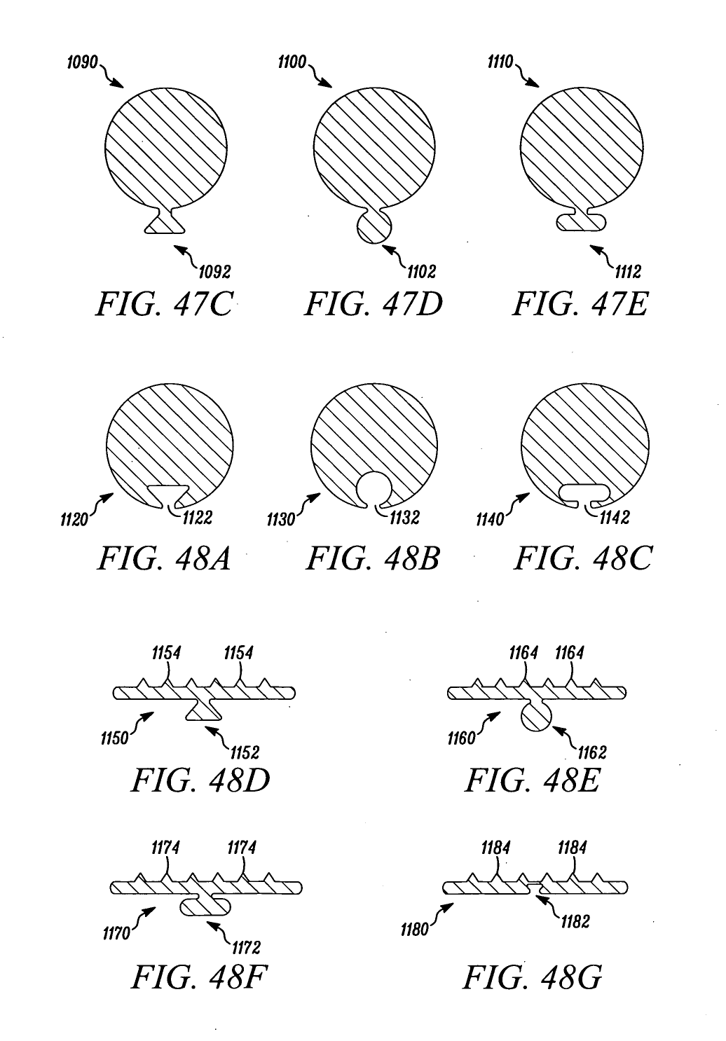

Referring to FIG. 47A, in one embodiment, a barrier device 1070 may include a central channel 1072, accessible by a slit 1076, and multiple flex grooves 1074. Multiple flex grooves 1074 may facilitate collapsing of barrier device 1070. In another embodiment, as in FIG. 47B, a barrier device 1080 may have a smooth, non-grooved surface and a central channel 1082, accessible by a slit 1086. Slit 1076, 1086 may facilitate coupling and decoupling of a tissue modification device with barrier device 1070, 1080. Referring to FIGS. 47C-47E, in various alternative embodiments, central channels 1072, 1082 may have any size or shape to allow passage of barrier devices 1070, 1080 over any of a number of guide members 1090, 1100, 1110 having variously shaped, protruding guide features 1092, 1102, 1112.

In alternative embodiments, and with reference now to FIGS. 48A-48C, guide members 1120, 1130, 1140 may alternatively include variously shaped grooves, impressions or tracks 1122, 1132, 1142 for accepting a protruding guide feature of a barrier device. FIGS. 48D-48G show various embodiments of tissue modification devices 1150, 1160, 1170, 1180, each having tissue modifying members 1154, 1164, 1174, 1184 and a differently shaped protruding guide feature 1152, 1162, 1172, 1182, such as a protrusion (FIGS. 48D-48F) or groove (FIG. 48G). Guide features 1152, 1162, 1172, 1182 may be used, in various embodiments, to facilitate guiding tissue modification devices 1150, 1160, 1170, 1180 along one or more guide members and/or barrier devices.

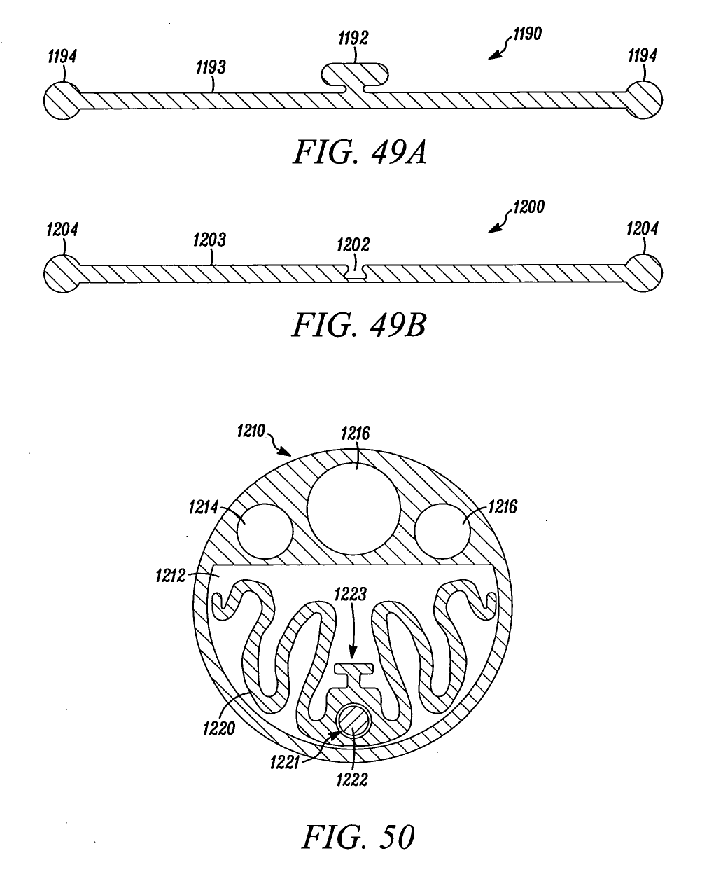

FIGS. 49A and 49B show two additional alternative embodiments of barrier devices 1190, 1200. Barrier device 1190 includes a protruding central guide feature 1192, a flat tissue protective portion 1193, and lateral support members 1194. Barrier device 1200 includes a central impression guide feature 1202, a flat tissue protective portion 1203, and lateral support members 1204.

As described immediately above, in any of a number of different embodiments, a barrier device may include one or more guide features. Such guide features may, in various embodiments, correspond with one or more guide features on a guide device or guide member for guiding the barrier member to a desired location and/or position in a patient. Alternative or additionally, one or more guide features on a barrier device may be used to facilitate guidance of one or more tissue modification devices along, over and/or through the barrier device. Thus, in some embodiments, a barrier member may include multiple guide features for guiding the barrier device and for guiding a tissue modification device. In other embodiments, the same guide feature(s) on a barrier device may be used to guide both the barrier device and a tissue modification device. Any suitable combination of guide feature(s) having any size, shape, pattern or the like may be used according to various embodiments.

FIG. 50 illustrates one embodiment of a delivery device 1210 for delivering a barrier device 1220 to a location in a patient. In this embodiment, barrier device 1220 includes a guidewire lumen 1221, through which a guidewire 1222 may extend, and a guide feature 1223, over which one or more tissue modification devices (not shown) may be passed. Optionally, delivery device 1210 may include a visualization lumen 1216, through which a visualization device may be passed, a suction lumen 1214, and an irrigation lumen 1216. In alternative embodiments, delivery device 1210 many have any of a number of suitable different configurations and features. For example, in one embodiment suction lumen 1214 and irrigation lumen 1216 may combined into one lumen, multiple visualization lumens 1216 may be included, and or the like.

As is mentioned above, in many of the described embodiments, a barrier device may include one or more pieces of material. Such material may include any suitable material or combination, and in some embodiments may comprise a polymer, such as latex, rubber (viton), nylon, silicone, polyetheretherketone (PEEK), polyetherketoneketone (PEKK), polytetrafluoroethylene (PTFE), polyurethane (Tecothane), Pebax (co, USA), polycarbonate, Delrin (DuPont, USA), high-density polyethylene (HDPE), low-density polyethylene (LDPE), high-molecular weight polyethylene (HMWPE), ultra-high-molecular weight polyethylene (UHMWPE), paraline coating, or the like. The material may be coated, laminated, impregnated, covered, or over-molded on a barrier device, or alternatively may be attached to a barrier device by adhesives or cements, thermal bonding techniques, with fasteners such as clasps or thread, or by forming pockets in the material which fit over ribs of the barrier.

In other embodiments, one or more conductive wires may be included in a barrier device, such that the wires may be disposed and selectively activated/exposed along either or both of a target tissue surface or a non-target tissue surface of the barrier device. In one embodiment, for example, wires may be coupled with lateral support members of a barrier device. Conductive wires may be used, for example, to stimulate and thus identify specific tissues, such as nerves, and/or to monitor the position/location of the barrier device by measuring impedance and/or imparting electrical currents to induce stimulation to the target tissue. In one embodiment, an array of wire contact points along a barrier device may be implemented and independently activated to verify that the barrier device is in a desired location/position.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. For example, in many of the embodiments described above, one or more abrasive tissue modifying members may be substituted for one or more bladed tissue modifying members or vice versa. These an many other modifications may be made to many of the described embodiments. Therefore, the foregoing description is provided primarily for exemplary purposes and should not be interpreted to limit the scope of the invention as it is set forth in the claims.

What is claimed is:

1. A method for preventing unwanted damage to tissue in a spine of a patient during a tissue modification procedure, the method comprising:
advancing a distal portion of a delivery device into an epidural space of the patient's spine;
exposing at least a portion of at least one barrier member out of the distal portion of the delivery device, wherein at least a portion of the barrier member is changeable from a collapsed configuration in the delivery device to an expanded configuration outside the delivery device;
positioning at least part of the exposed barrier member between target tissue and non-target tissue in the spine; and
performing at least one tissue modification procedure on the target tissue, using at least one tissue modification device, wherein at least part of the barrier member is disposed between the at least one tissue modification device and the non-target tissue to prevent unwanted damage to the non-target tissue.

2. A method as in claim 1, further comprising advancing the at least one barrier member through the delivery device.

3. A method as in claim 2, wherein the at least one barrier member is advanced through the delivery device over at least one guide member selected from the group consisting of guidewires, rails, tracks and lengthwise impressions.

4. A method as in claim 3, wherein at least two guide members are used, the method further comprising orienting the barrier member relative to the target and non-target tissues using the guide members.

5. A method as in claim 1, wherein exposing the barrier member from the delivery device causes it to change automatically from the collapsed to the expanded configuration.

6. A method as in claim 1, further comprising causing at least part of the exposed barrier member to change from the collapsed to the expanded configuration.

7. A method as in claim 6, wherein causing the barrier member to change from the collapsed to the expanded configuration comprises performing an action selected from the list consisting of pulling one or more actuators to remove one or more constraining members, pushing a rigid rod to remove one or more constraining members, changing the positions of multiple interacting members in the barrier member, unfolding the barrier, unfurling the barrier, stretching the barrier between two or more scaffolding members, advancing a proximal portion of the barrier into a distal portion, retracting a distal portion of the barrier into a proximal portion, activating a spring mechanism, inflating one or more bladders disposed within the barrier member and exposing the barrier member to fluid.

8. A method as in claim 1, wherein positioning the at least one barrier member comprises passing at least a distal portion of the barrier member at least partially into an intervertebral foramen.

9. A method as in claim 1, wherein performing at least one procedure on the patient's spine comprises using at least one surgical tool selected from the list consisting of a rongeur, a curette, a scalpel, one or more cutting blades, a scissors, a forceps, a probe, a rasp, a file, an abrasive element, one or more small planes, an electrosurgical device, a bipolar electrode, a unipolar electrode, a thermal electrode a rotary powered mechanical shaver, a reciprocating powered mechanical shaver, a powered mechanical burr, a laser, an ultrasound crystal, a cryogenic probe, and a pressurized water jet.

10. A method as in claim 9, further comprising using the barrier member to guide the at least one surgical tool to a position for performing the at least one procedure.

11. A method as in claim 1, wherein performing at least one procedure comprises:
contacting at least one tissue modifying member of the tissue modification device with the target tissue;
applying at least one of anchoring force and tensioning force to proximal and distal portions of the tissue modification device to urge the tissue modifying member(s) against the target tissue; and
modifying the target tissue using the tissue modifying member(s).

12. A method as in claim 1, further comprising applying force to the barrier member to urge at least one tissue modifying member of a tissue modification device against the target tissue.

13. A method as in claim 12, wherein the at least one barrier member comprises an elongate, at least partially flexibly barrier member, and wherein applying force to the barrier member comprises applying at least one of anchoring force and tensioning force to proximal and distal portions of the barrier member.

14. A method as in claim 1, wherein the non-target tissue comprises at least one of nerve, blood vessel, intervertebral disc, bone and ligament.

15. A method as in claim 1, further comprising visualizing an area for performing the at least one procedure, using an elongate, flexible visualization device extended through a lumen in at least one of the delivery device and the barrier member.

16. A method as in claim 1, further comprising delivering an electric current to at least one of the target and non-target tissue via at least one of the barrier member, the delivery device and the tissue modification device.

17. A method as in claim 16, further comprising monitoring an effect of the electric current on the tissue by observing at least one of patient feedback, muscle or limb movement, electromyelographic (EMG) monitoring and somatosensory evoked potentials (SSEP).

18. A method as in claim 1, further comprising introducing fluid to an area for performing the at least one procedure through one or more lumens in at least one of the delivery device and the barrier member.

19. A method as in claim 1, further comprising removing tissue debris from the patient, using the barrier member as a conduit.

20. A method for preventing unwanted damage to tissue in a spine of a patient during a tissue modification procedure, the method comprising:
advancing at least a distal portion of at least one barrier member over at least one guide member into an epidural space of the patient's spine;
positioning at least an expanded portion of the barrier member between target tissue and non-target tissue; and
performing at least one tissue modification procedure on the target tissue, using at least one tissue modification device, wherein at least part of the barrier member is disposed between the at least one tissue modification device and the non-target tissue to prevent unwanted damage to the non-target tissue.

21. A method as in claim 20, wherein advancing the barrier member comprises advancing through a delivery device.

22. A method as in claim 20, wherein the barrier member is advanced over at least two guide members, the method further comprising orienting the barrier member relative to the target and non-target tissues using the guide members.

23. A method as in claim 20, further comprising expanding at least part of the barrier member from a collapsed configuration to an expanded configuration.

24. A method as in claim 23, wherein expanding at least part of the barrier member comprises performing an action selected from the list consisting of pulling one or more actuators to remove one or more constraining members, pushing a rigid rod to remove one or more constraining members, changing the positions of multiple interacting members in the barrier member, unfolding the barrier, unfurling the barrier, stretching the barrier between two or more scaffolding members, advancing a proximal portion of the barrier into a distal portion, retracting a distal portion of the barrier into a proximal portion, activating a spring mechanism, inflating one or more bladders disposed within the barrier member and exposing the barrier member to fluid.

25. A method as in claim 20, wherein positioning the at least one barrier member comprises passing at least the distal portion of the barrier member at least partially into an intervertebral foramen.

26. A method as in claim 20, wherein performing at least one procedure on the patient's spine comprises using at least one surgical tool selected from the list consisting of a rongeur, a curette, a scalpel, one or more cutting blades, a scissors, a forceps, a probe, a rasp, a file, an abrasive element, one or more small planes, an electrosurgical device, a bipolar electrode, a unipolar electrode, a thermal electrode a rotary powered mechanical shaver, a reciprocating powered mechanical shaver, a powered mechanical burr, a laser, an ultrasound crystal, a cryogenic probe, and a pressurized water jet.

27. A method as in claim 26, further comprising using the barrier member to guide the at least one surgical tool to a position for performing the at least one procedure.

28. A method as in claim 20, wherein performing at least one procedure comprises:
contacting at least one tissue modifying member of the tissue modification device with the target tissue;
applying at least one of anchoring force and tensioning force proximal and distal portions of the tissue modification device to urge the tissue modifying member(s) against the target tissue; and
modifying the target tissue using the tissue modifying member(s).

29. A method as in claim 20, further comprising applying force to the barrier member to urge at least one tissue modifying member of a tissue modification device against the target tissue.

30. A method as in claim 29, wherein the at least one barrier member comprises an elongate, at least partially flexibly barrier member, and wherein applying force to the barrier member comprises applying at least one of anchoring force and tensioning force to proximal and distal portions of the barrier member.

31. A method as in claim 20, wherein the non-target tissue comprises at least one of nerve, blood vessel, intervertebral disc, bone and ligament.

32. A method as in claim 20, further comprising visualizing an area for performing the at least one procedure, using an elongate, flexible visualization device extended through a lumen in at least one of a barrier member delivery device and the barrier member.

33. A method as in claim 20, further comprising delivering an electric current to at least one of the target and non-target tissue via at least one of the barrier member, the delivery device and the tissue modification device.

34. A method as in claim 33, further comprising monitoring an effect of the electric current on the tissue by observing at least one of patient feedback, muscle or limb movement, electromyelographic (EMG) monitoring and somatosensory evoked potentials (SSEP).

35. A method as in claim 20, further comprising introducing fluid to an area for performing the at least one procedure through one or more lumens in at least one of a barrier member delivery device and the barrier member.

36. A method as in claim 20, further comprising removing tissue debris from the patient, using the barrier member as a conduit.

37. A method for preventing unwanted damage to tissue of a patient during a tissue modification procedure, the method comprising:
advancing at least a distal portion of a delivery device into the patient and to a position between or adjacent target tissue and non-target tissue;
advancing at least a distal portion of at least one barrier member over at least one guide member to a position between or adjacent target tissue and non-target tissue in the patient;
exposing at least a portion of the at least one barrier member out of the distal portion of the delivery device, wherein at least a portion of the barrier member is changeable from a collapsed configuration in the delivery device to an expanded configuration outside the delivery device; and
performing at least one tissue modification procedure on the target tissue, using at least one tissue modification device, wherein at least part of the barrier member is disposed between the at least one tissue modification device and the non-target tissue to prevent unwanted damage to the non-target tissue.

38. A barrier device for preventing unwanted damage to tissue of a patient during a tissue modification procedure, the device comprising:
at least one shape changing portion changeable from a collapsed configuration, to facilitate passage into the patient, to an expanded configuration, to facilitate protection of non-target tissue;
at least one elongate portion extending beyond the shape changing portion, the elongate portion having a low profile to facilitate passage of the barrier device into the patient and a length sufficient to extend from an opening on the patient's skin to an area at or near target and non-target tissues; and
at least one guide feature extending along at least a portion of the barrier to allow the barrier to be passed into the patient over at least one guide member,
wherein the barrier device has an overall length sufficient to pass from a first opening on the patient's skin and between the target and non-target tissues.

39. A device as in claim 38, wherein the device has an overall length, size and configuration to allow it to extend from the first opening on the patient's skin, into an epidural space of the patient's spine, and between target and non-target tissues in the spine.

40. A device as in claim 39, wherein the overall length of the device is sufficient to allow it to further extend from outside the patient, through the first opening, between the target and non-target tissues, and out the patient through a second opening on the patients skin.

41. A device as in claim 38, wherein the at least one elongate portion comprises:
a proximal elongate portion extending from a proximal end of the shape changing portion; and
a distal elongate portion extending from a distal end of the shape changing portion.

42. A device as in claim 38, further comprising a barrier delivery device through which the barrier device may be passed into the patient.

43. A device as in claim 42, wherein the barrier delivery device is selected from the group consisting of sheaths and catheters.

44. A device as in claim 42, wherein the shape changing portion changes shape automatically when released from the barrier delivery device.

45. A device as in claim 44, wherein the shape changing portion comprises a super-elastic or shape memory material.

46. A device as in claim 44, wherein the shape changing portion is foldable to assume the collapsed configuration and unfolds to assume the expanded configuration upon release from the delivery device.

47. A device as in claim 38, further comprising at least one actuator coupled with the shape changing portion to change it from its collapsed to its expanded configuration.

48. A device as in claim 47, wherein the at least one actuator is selected from the group consisting of zippers, a tear strips, pull wires, expandable scaffolds, inflatable bladders, umbrellas and expandable stents.

49. A device as in claim 47, wherein the at least one actuator extends from one end of the shape changing portion.

50. A device as in claim 47, wherein the at least one actuator comprises two actuators extending from opposite ends of the shape changing portion.

51. A device as in claim 38, wherein the at least one guide feature is selected from the group consisting of guidewire lumens, rails, tracks and lengthwise impressions.

52. A device as in claim 51, wherein the at least one guide feature comprises at least two guidewire lumens extending along at least the shape changing portion, and wherein an area of the shape changing portion between the two guidewire lumens has a sufficient stiffness to prevent the shape changing portion from twisting.

53. A device as in claim 38, further comprising at least one additional guide feature configured to facilitate guidance of one or more tissue modification devices along the barrier member.

54. A device as in claim 38, wherein the at least one guide feature is configured to allow passage of at least one tissue modification device along the barrier member.

55. A device as in claim 38, further comprising at least one conductive electrode coupled with the barrier device for delivering electric current to at least one of the target and non-target tissue.

56. A device as in claim 55, further comprising at least one monitoring device coupled with the barrier device for monitoring an effect of the electric current on the tissue, the monitoring device selected from the group consisting of EMG monitoring devices and SSEP monitoring devices.

57. A device as in claim 38, wherein at least the shape changing portion of the barrier device comprises:
a front surface for facing one or more tissue modification devices for performing a procedure in the spine; and
a back surface for facing one or more non-target tissues.

58. A device as in claim 57, wherein the front and back surfaces have different shapes to facilitate positioning of the barrier member between target and non-target tissues to protect the non-target tissue.

59. A device as in claim 38, wherein the shape changing portion comprises a window and the at least one elongate portion comprises two elongate tubular extensions extending from opposite ends of the window.

60. A device as in claim 38, wherein the shape changing portion comprises an expandable scaffold.

61. A device as in claim 60, wherein the shape changing portion further comprises one or more pieces of material coupled with the scaffold.

62. A device as in claim 38, wherein the shape changing portion comprises:
a plurality of longitudinal support members; and
one or more pieces of material coupled with support members.

63. A device as in claim 38, wherein the shape changing portion comprises at least one of a hydrogel material, a wire mesh, an expandable stent and an inflatable bladder.
